(19) United States
(12) Patent Application Publication
(10) Pub. No.: US 2025/0325516 A1
(43) Pub. Date: Oct. 23, 2025

(54) SEROTONIN ANALOGUES FOR USE IN TREATING METALLOPTOSIS-ASSOCIATED DISORDERS

(71) Applicants: INSTITUT CURIE, PARIS (FR); INSTITUT NATIONAL DE LA SANTÉ ET DE LA RECHERCHE MÉDICALE, PARIS (FR); CENTRE NATIONAL DE LA RECHERCHE SCIENTIFIQUE, PARIS (FR); UNIVERSITÉ PARIS CITÉ, PARIS (FR); INSTITUT GUSTAVE ROUSSY, VILLEJUIF (FR); PARIS SCIENCES ET LETTRES, PARIS (FR); FONDATION IMAGINE - INSTITUT DES MALADIES GENETIQUES, PARIS (FR); ASSISTANCE PUBLIQUE - HÔPITAUX DE PARIS, PARIS (FR)

(72) Inventors: RAPHAËL RODRIGUEZ, IVRY-SUR-SEINE (FR); SEBASTIAN MULLER, IVRY-SUR-SEINE (FR); TATIANA CAÑEQUE, IVRY-SUR-SEINE (FR); LUDOVIC COLOMBEAU, ORLÉANS (FR); LEISHEMBA KHUMAN THOIDINGJAM, PARIS (FR); FRANCINE CÔTÉ, PARIS (FR)

(21) Appl. No.: 18/881,803

(22) PCT Filed: Feb. 14, 2023
(86) PCT No.: PCT/EP2023/053677

§ 371 Date: Jan. 7, 2025

(30) Foreign Application Priority Data
Jul. 8, 2022 (EP) 22306022.9
Aug. 17, 2022 (WO) PCT/EP2022/073004

(51) Int. Cl. A61K 31/4045; A61K 31/454; C07D 209/16; C07D 209/30; C07D 401/06; C07D 403/06; C07D 405/12; C07D 405/14

(52) U.S. Cl. CPC A61K 31/4045; A61K 31/454; C07D 209/16; C07D 209/30; C07D 401/06; C07D 403/06; C07D 405/12; C07D 405/14

(57) ABSTRACT

The present invention relates to serotonin derivatives of general formula (I), and their use in the pharmaceutical field, in particular for preventing or treating metalloptosis associated disorders. The invention also relates to new serotonin derivatives of general formula (I) and their use for preventing or treating iron- and/or copper-associated disorders.

### FIELD OF THE INVENTION

The present invention is in the field of medicine. The present invention relates to new serotonin analogues and their use as drugs, in particular for preventing and/or treating metalloptosis associated disorders.

### BACKGROUND OF THE INVENTION

Beyond classical apoptosis, several forms of regulated cell death (RCD) have been identified. These RCD subroutines differ in the initiating stimuli, intermediate activation events, and end effectors. Various metals can induce regulated cell death, i.e. metalloptosis, through different subroutines. Metal ions are essential micronutrients, but either insufficient or excessive abundance of metals can trigger cell death. Metalloptosis includes in particular ferroptosis and cuproptosis that are dependent on iron and copper respectively.

Ferroptosis is a type of programmed cell death dependent on iron and characterized by the accumulation of lipid peroxides, and is genetically and biochemically distinct from other forms of regulated cell death such as apoptosis. Ferroptosis is initiated by the failure of the glutathione-dependent antioxidant defenses, resulting in unchecked lipid peroxidation and eventual cell death. The research field of ferroptosis has seen exponential growth over the past few years, since the term was coined in 2012. This unique modality of cell death, driven by iron-dependent phospholipid peroxidation, is regulated by multiple cellular metabolic pathways, including redox homeostasis, iron handling, mitochondrial activity and metabolism of amino acids, lipids and sugars, in addition to various signalling pathways relevant to disease (Xuejun Jiang et al., Nature Reviews Molecular Cell Biology vol. 22, 266-282 (25.01.2021)). Numerous organ injuries and degenerative pathologies are driven by ferroptosis. Recent studies have shown that ferroptosis is closely related to the pathophysiological processes of many diseases, such as tumors, nervous system diseases, ischemia-reperfusion injury, kidney injury, and blood diseases (Jie Li et al., Cell Death & Disease vol. 11, 88 (03.02.2020)).

A recent study showed that intracellular copper (Cu) induces a novel form of RCD that is different from oxidative stress-related cell death (e.g., apoptosis, ferroptosis, and necroptosis) and has been termed “cuproptosis” (Tsvetkov, P. et al. Science 375, 1254-1261 (2022)). In contrast, mitochondrial stress, especially the aggregation of lipoylated mitochondrial enzymes and the loss of Fe—S cluster proteins, ignites cuproptosis.

There is thus a need to find new compounds activating or blocking the metalloptosis pathways to alleviate the progression of the disease, which provides a promising therapeutic strategy for many diseases.

### SUMMARY OF THE INVENTION

The inventors discovered and developed serotonin analogues capable of activating or blocking the metalloptosis pathways, and thus preventing and/or treating diseases associated to metalloptosis. The inventors also discovered that these serotonin analogues are effective for preventing or treating iron- and/or copper-associated disorders.

Accordingly, the invention relates to a compound of general formula (I) for use in preventing or treating metalloptosis associated disorders, or a pharmaceutically acceptable salt and/or solvate thereof, wherein formula (I) is:

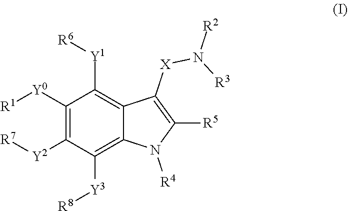

wherein
R¹, R², R³ and R⁴ are independently selected in the group consisting of H, —OH, —C(O)OR⁹—, —S(O)₂R¹⁰—, —C(O)R⁹—, —S(O)R¹⁰—, —NR¹¹R¹²—, optionally substituted (C₁-C₂₄)alkyl, optionally substituted (C₂-C₂₄)alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl, provided that at least one of R¹, R² and R³ is not H;
or
R¹ and R⁴ are independently selected in the group consisting of H, —OH, —C(O)OR⁹—, —S(O)₂R¹⁰—, —C(O)R⁹—, —S(O)R¹⁰—, —NR¹¹R¹²—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl, and R² and R³ form together with the nitrogen atom to which they bound an optionally substituted heterocycloalkyl;
X is selected in the group consisting of (C₁-C₁₂)alkyl, —O—(C₁-C₁₂)alkyl-, —C(O)—, —C(O)—(C₁-C₁₂)alkyl-, —NH—C(O)—(C₁-C₁₂)alkyl-, —S—(C₁-C₁₂)alkyl-, —S(O)—(C₁-C₁₂)alkyl-, and —S(O)₂—(C₁-C₁₂)alkyl-, the (C₁-C₁₂)alkyl groups being optionally substituted and/or optionally interrupted by an heteroatom;
   Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴—, (C₁-C₂₄)alkyl, —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴—, —NH—C(O)—NH—, —NH—S(O)—NH—, —NH—S(O₂)—NH—, —S—C(O)—NH—, —O—C(O)—NH—, —O—S(O)—NH—, —O—S(O₂)—NH—, —S—CH₂—C(O)—NH—, —S—CH₂—S(O)—NH—, —S—CH₂—S(O₂)—NH—, —S(O)—CH₂—C(O)—NH—, —S(O₂)—CH₂—C(O)—NH—, —O—CH₂—C(O)—NH—, —O—CH₂—S(O)—NH—, —O—CH₂—S(O₂)—NH—, —NH—CH₂—C(O)—NH—, —NH—CH₂—S(O)—NH—, —NH—CH₂—S(O₂)—NH—, —O—(CH₂)₂—NH—C(O)—, —O—(CH₂)₂—N(CH₃)—C(O)—, —O—(CH₂)₂—NH—S(O)—, —O—(CH₂)₂—N(CH₃)—S(O)—, —O—(CH₂)₂—NH—S(O₂)—, —O—(CH₂)₂—N(CH₃)—S(O₂)—, —S—(CH₂)₂—NH—C(O)—, —S—(CH₂)₂—N(CH₃)—C(O)—, —S—(CH₂)₂—NH—S(O)—, —S—(CH₂)₂—N(CH₃)—S(O)—, —S—(CH₂)₂—NH—S(O₂)—, —S—(CH₂)₂—N(CH₃)—S(O₂)—, —NH—(CH₂)₂—NH—C(O)—, —NH—(CH₂)₂—N(CH₃)—C(O)—, —NH—(CH₂)₂—NH—S(O)—, —NH—(CH₂)₂—N(CH₃)—S(O)—, —NH—(CH₂)₂—NH—S(O₂)—, —NH—(CH₂)₂—N(CH₃)—S(O₂)—, —O—CH₂—CH(CH₂—OH)—NH—C(O)—, —O—CH₂—CH(CH₂—OCH₃)—NH—C(O)—, —O—CH₂—CH(CH₃)—NH—C(O)—, —O—CH(CH₃)—CH₂—NH—C(O)—, —O—CH₂—CH(CH₂—OH)—NH—S(O)—, —O—CH₂—CH(CH₂—OCH₃)—NH—S(O)—, —O—CH₂—CH(CH₃)—NH—S(O)—, —O—CH(CH₃)—CH₂—NH—S(O)—, —O—CH₂—CH(CH₂—OH)—NH—S(O₂)—, —O—CH₂—CH(CH₂—OCH₃)—NH—S(O₂)—, —O—CH₂—CH(CH₃)—NH—S(O₂)—, —O—CH(CH₃)—CH₂—NH—S(O₂)—, —S—CH₂—CH(CH₂—OH)—NH—C(O)—, —S—CH₂—CH(CH₂—OCH₃)—NH—C(O)—, —S—CH₂—CH(CH₃)—NH—C(O)—, —S—CH(CH₃)—CH₂—NH—C(O)—, —S—CH₂—CH(CH₂—OH)—NH—S(O)—, —S—CH₂—CH(CH₂—OCH₃)—NH—S(O)—, —S—CH₂—CH(CH₃)—NH—S(O)—, —S—CH(CH₃)—CH₂—NH—S(O)—, —S—CH₂—CH(CH₂—OH)—NH—S(O₂)—, —S—CH₂—CH(CH₂—OCH₃)—NH—S(O₂)—, —S—CH₂—CH(CH₃)—NH—S(O₂)—, —S—CH(CH₃)—CH₂—NH—S(O₂)—, —NH—CH₂—CH(CH₂—OH)—NH—C(O)—, —NH—CH₂—CH(CH₂—OCH₃)—NH—C(O)—, —NH—CH₂—CH(CH₃)—NH—C(O)—, —NH—CH(CH₃)—CH₂—NH—C(O)—, —NH—CH₂—CH(CH₂—OH)—NH—S(O)—, —NH—CH₂—CH(CH₂—OCH₃)—NH—S(O)—, —NH—CH₂—CH(CH₃)—NH—S(O)—, —NH—CH(CH₃)—CH₂—NH—S(O)—, —NH—CH₂—CH(CH₂—OH)—NH—S(O₂)—, —NH—CH₂—CH(CH₂—OCH₃)—NH—S(O₂)—, —NH—CH₂—CH(CH₃)—NH—S(O₂)—, —NH—CH(CH₃)—CH₂—NH—S(O₂)—, —O—(CH₂)₂—NH—, —S—(CH₂)₂—NH—, and —NH—(CH₂)₂—NH—; the (C₁-C₂₄)alkyl groups being optionally substituted; and
R⁵, R⁶, R⁷ and R⁸ are independently selected in the group consisting of H, halogen, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl; and
R⁹ to R¹⁴ are independently selected in the group consisting of optionally substituted (C₁-C₁₂)alkyl, optionally substituted (C₂-C₁₂) alkenyl, optionally substituted (C₂-C₁₂) alkynyl, optionally substituted aryl.

Preferably, R⁹ is selected in the group consisting of optionally substituted (C₁-C₁₂)alkyl, optionally substituted (C₂-C₁₂) alkenyl, optionally substituted (C₂-C₁₂) alkynyl, and aryl optionally substituted with a (C₁-C₆)alkyl; and
R¹⁰ to R¹⁴ are independently selected in the group consisting of optionally substituted (C₁-C₁₂)alkyl, optionally substituted (C₂-C₁₂) alkenyl, optionally substituted (C₂-C₁₂) alkynyl, optionally substituted aryl.

In a particular aspect, the metalloptosis associated disorders are disorders associated to ferroptosis and/or cuproptosis.

In a particular aspect, the metalloptosis associated disorders are selected among Hemorrhagic stroke; Ischemic Heart Injury; Ischemic Renal Injury; Ischaemia-reperfusion injury (IRI); Neurodegenerative diseases selected from Huntington Disease, Dementia, and amyotrophic lateral sclerosis (ALS); aging related diseases or disorders; sepsis; ishemia-reperfusion; and Diabetes.

The invention also relates to a compound of general formula (I):

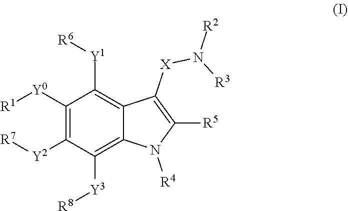

or a pharmaceutically acceptable salt and/or solvate thereof,
wherein
R¹, R², R³ and R⁴ are independently selected in the group consisting of H, OH, —C(O)OR⁹—, —S(O)₂R¹⁰—, —C(O)R⁹—, —S(O)R¹⁰—, —NR¹¹R¹²—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl, provided that at least one of R² and R³ is not H;
or
R¹ and R⁴ are independently selected in the group consisting of H, OH, —C(O)OR⁹—, —S(O)₂R¹⁰—, —C(O)R⁹—, —S(O)R¹⁰—, —NR¹¹R¹², optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl, and R² and R³ with the nitrogen atom to which they are bound form together a nitrogen-heterocycloalkyl optionally substituted with at least one substituent selected in the group consisting of halogen; CN; NO₂; (C₁-C₆)alkyl; (C₁-C₅)haloalkyl; (C₁-C₅)haloalkoxy, (C₁-C₆)halothioalkoxy, optionally substituted aryl (in particular optionally substituted with one or two (C₁-C₆)alkyl or NO₂); heteroaryl; oxo; NR^{a}R^{b}; COR^{c}; CO₂R^{d}; CONR^{e}R^{f}; OR^{g}; SR^{h}; SO₂Rⁱ; SO₂NR^{j}R^{k}; NR^{l}COR^{m}; RⁿNR^{O}R^{P}; NR^{q}SO₂R^{r}; wherein R^{a} to R^{r} are, independently of one another, H, (C₁-C₅)alkyl, (C₁-C₅)haloalkyl, aryl, (C₁-C₅)alkyl-aryl, heteroaryl or (C₁-C₆)alkyl-heteroaryl; in particular consisting of halogen, CN, NO₂, (C₁-C₅)alkyl, (C₁-C₆)haloalkyl, oxo, NR^{a}R^{b}, COR^{c}, C(O)OR^{d}, CONR^{e}R^{f}, and OR^{g}, wherein R^{a} to R^{g} are, independently of one another, H or (C₁-C₆)alkyl;
X is selected in the group consisting of (C₁-C₁₂)alkyl, —O—(C₁-C₁₂)alkyl-, —C(O)—, —C(O)—(C₁-C₁₂)alkyl-, —NH—C(O)—(C₁-C₁₂)alkyl-, —S—(C₁-C₁₂)alkyl-, —S(O)—(C₁-C₁₂)alkyl-, and —S(O)₂—(C₁-C₁₂)alkyl-; the (C₁-C₁₂)alkyl groups being optionally substituted and/or optionally interrupted by an heteroatom;
Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴—, (C₁-C₂₄)alkyl, —NH—C(O)—NH—, —NH—S(O)—NH—, —NH—S(O₂)—NH—, —S—C(O)—NH—, —O—C(O)—NH—, —O—S(O)—NH—, —O—S(O₂)—NH—, —S—CH₂—C(O)—NH—, —S—CH₂—S(O)—NH—, —S—CH₂—S(O₂)—NH—, —S(O)—CH₂—C(O)—NH—, —S(O₂)—CH₂—C(O)—NH—, —O—CH₂—C(O)—NH—, —O—CH₂—S(O)—NH—, —O—CH₂—S(O₂)—NH—, —NH—CH₂—C(O)—NH—, —NH—CH₂—S(O)—NH—, —NH—CH₂—S(O₂)—NH—, —O—(CH₂)₂—NH—C(O)—, —O—(CH₂)₂—N(CH₃)—C(O)—, —O—(CH₂)₂—NH—S(O)—, —O—(CH₂)₂—N(CH₃)—S(O)—, —O—(CH₂)₂—NH—S(O₂)—, —O—(CH₂)₂—N(CH₃)—S(O₂)—, —S—(CH₂)₂—NH—C(O)—, —S—(CH₂)₂—N(CH₃)—C(O)—, —S—(CH₂)₂—NH—S(O)—, —S—(CH₂)₂—N(CH₃)—S(O)—, —S—(CH₂)₂—NH—S(O₂)—, —S—(CH₂)₂—N(CH₃)—S(O₂)—, —NH—(CH₂)₂—NH—C(O)—, —NH—(CH₂)₂—N(CH₃)—C(O)—, —NH—(CH₂)₂—NH—S(O)—, —NH—(CH₂)₂—N(CH₃)—S(O)—, —NH—(CH₂)₂—NH—S(O₂)—, —NH—(CH₂)₂—N(CH₃)—S(O₂)—, —O—CH₂—CH(CH₂—OH)—NH—C(O)—, —O—CH₂—CH(CH₂—OCH₃)—NH—C(O)—, —O—CH₂—CH(CH₃)—NH—C(O)—, —O—CH(CH₃)—CH₂—NH—C(O)—, —O—CH₂—CH(CH₂—OH)—NH—S(O)—, —O—CH₂—CH(CH₂—OCH₃)—NH—S(O)—, —O—CH₂—CH(CH₃)—NH—S(O)—, —O—CH(CH₃)—CH₂—NH—S(O)—, —O—CH₂—CH(CH₂—OH)—NH—S(O₂)—, —O—CH₂—CH(CH₂—OCH₃)—NH—S(O₂)—, —O—CH₂—CH(CH₃)—NH—S(O₂)—, —O—CH(CH₃)—CH₂—NH—S(O₂)—, —S—CH₂—CH(CH₂—OH)—NH—C(O)—, —S—CH₂—CH(CH₂—OCH₃)—NH—C(O)—, —S—CH₂—CH(CH₃)—NH—C(O)—, —S—CH(CH₃)—CH₂—NH—C(O)—, —S—CH₂—CH(CH₂—OH)—NH—S(O)—, —S—CH₂—CH(CH₂—OCH₃)—NH—S(O)—, —S—CH₂—CH(CH₃)—NH—S(O)—, —S—CH(CH₃)—CH₂—NH—S(O)—, —S—CH₂—CH(CH₂—OH)—NH—S(O)—, —S—CH₂—CH(CH₂—OCH₃)—NH—S(O₂)—, —S—CH₂—CH(CH₃)—NH—S(O₂)—, —S—CH(CH₃)—CH₂—NH—S(O₂)—, —NH—CH₂—CH(CH₂—OH)—NH—C(O)—, —NH—CH₂—CH(CH₂—OCH₃)—NH—C(O)—, —NH—CH₂—CH(CH₃)—NH—C(O)—, —NH—CH(CH₃)—CH₂—NH—C(O)—, —NH—CH₂—CH(CH₂—OH)—NH—S(O)—, —NH—CH₂—CH(CH₂—OCH₃)—NH—S(O)—, —NH—CH₂—CH(CH₃)—NH—S(O)—, —NH—CH(CH₃)—CH₂—NH—S(O)—, —NH—CH₂—CH(CH₂—OH)—NH—S(O₂)—, —NH—CH₂—CH(CH₂—OCH₃)—NH—S(O₂)—, —NH—CH₂—CH(CH₃)—NH—S(O₂)—, —NH—CH(CH₃)—CH₂—NH—S(O₂)—, —O—(CH₂)₂—NH—, —S—(CH₂)₂—NH—, and —NH—(CH₂)₂—NH—; the (C₁-C₂₄)alkyl groups being optionally substituted; and
R⁵, R⁶, R⁷ and R⁸ are independently selected in the group consisting of H, halogen, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl; and
R⁹ to R¹⁴ are independently selected in the group consisting of optionally substituted (C₁-C₁₂)alkyl, optionally substituted (C₂-C₁₂) alkenyl, optionally substituted (C₂-C₁₂) alkynyl, optionally substituted aryl;
provided that when R⁵, R⁶, R⁷ and R⁸ are H, Y⁰ is —O—, and Y¹, Y² and Y³ are absent, then R¹, R² and R³ are not an optionally substituted (C₂-C₂₄) alkynyl.

Preferably, Y¹, Y² and Y³ are independently absent or selected in the following list and Y⁰ is selected in the following list; the list being: —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴—, (C₁-C₂₄)alkyl, —NH—C(O)—NH—, —NH—S(O)—NH—, —NH—S(O₂)—NH—, —S—C(O)—NH—, —O—C(O)—NH—, —O—S(O)—NH—, —O—S(O₂)—NH—, —S—CH₂—C(O)—NH—, —S—CH₂—S(O)—NH—, —S—CH₂—S(O₂)—NH—, —S(O)—CH₂—C(O)—NH—, —S(O₂)—CH₂—C(O)—NH—, —O—CH₂—C(O)—NH—, —O—CH₂—S(O)—NH—, —O—CH₂—S(O₂)—NH—, —NH—CH₂—C(O)—NH—, —NH—CH₂—S(O)—NH—, —NH—CH₂—S(O₂)—NH—, —O—(CH₂)₂—NH—C(O)—, —O—(CH₂)₂—N(CH₃)—C(O)—, —O—(CH₂)₂—NH—S(O)—, —O—(CH₂)₂—N(CH₃)—S(O)—, —O—(CH₂)₂—NH—S(O₂)—, —O—(CH₂)₂—N(CH₃)—S(O₂)—, —S—(CH₂)₂—NH—C(O)—, —S—(CH₂)₂—N(CH₃)—C(O)—, —S—(CH₂)₂—NH—S(O)—, —S—(CH₂)₂—N(CH₃)—S(O)—, —S—(CH₂)₂—NH—S(O₂)—, —S—(CH₂)₂—N(CH₃)—S(O₂)—, —NH—(CH₂)₂—NH—C(O)—, —NH—(CH₂)₂—N(CH₃)—C(O)—, —NH—(CH₂)₂—NH—S(O)—, —NH—(CH₂)₂—N(CH₃)—S(O)—, —NH—(CH₂)₂—NH—S(O₂)—, —NH—(CH₂)₂—N(CH₃)—S(O₂)—, —O—CH₂—CH(CH₂—OH)—NH—C(O)—, —O—CH₂—CH(CH₂—OCH₃)—NH—C(O)—, —O—CH₂—CH(CH₃)—NH—C(O)—, —O—CH(CH₃)—CH₂—NH—C(O)—, —O—CH₂—CH(CH₂—OH)—NH—S(O)—, —O—CH₂—CH(CH₂—OCH₃)—NH—S(O)—, —O—CH₂—CH(CH₃)—NH—S(O)—, —O—CH(CH₃)—CH₂—NH—S(O)—, —O—CH₂—CH(CH₂—OH)—NH—S(O₂)—, —O—CH₂—CH(CH₂—OCH₃)—NH—S(O₂)—, —O—CH₂—CH(CH₃)—NH—S(O₂)—, —O—CH(CH₃)—CH₂—NH—S(O₂)—, —S—CH₂—CH(CH₂—OH)—NH—C(O)—, —S—CH₂—CH(CH₂—OCH₃)—NH—C(O)—, —S—CH₂—CH(CH₃)—NH—C(O)—, —S—CH(CH₃)—CH₂—NH—C(O)—, —S—CH₂—CH(CH₂—OH)—NH—S(O)—, —S—CH₂—CH(CH₂—OCH₃)—NH—S(O)—, —S—CH₂—CH(CH₃)—NH—S(O)—, —S—CH(CH₃)—CH₂—NH—S(O)—, —S—CH₂—CH(CH₂—OH)—NH—S(O₂)—, —S—CH₂—CH(CH₂—OCH₃)—NH—S(O₂)—, —S—CH₂—CH(CH₃)—NH—S(O₂)—, —S—CH(CH₃)—CH₂—NH—S(O₂)—, —NH—CH₂—CH(CH₂—OH)—NH—C(O)—, —NH—CH₂—CH(CH₂—OCH₃)—NH—C(O)—, —NH—CH₂—CH(CH₃)—NH—C(O)—, —NH—CH(CH₃)—CH₂—NH—C(O)—, —NH—CH₂—CH(CH₂—OH)—NH—S(O)—, —NH—CH₂—CH(CH₂—OCH₃)—NH—S(O)—, —NH—CH₂—CH(CH₃)—NH—S(O)—, —NH—CH(CH₃)—CH₂—NH—S(O)—, —NH—CH₂—CH(CH₂—OH)—NH—S(O₂)—, —NH—CH₂—CH(CH₂—OCH₃)—NH—S(O₂)—, —NH—CH₂—CH(CH₃)—NH—S(O₂)—, —NH—CH(CH₃)—CH₂—NH—S(O₂)—, —O—(CH₂)₂—NH—, —S—(CH₂)₂—NH—, and —NH—(CH₂)₂—NH—; the (C₁-C₂₄)alkyl groups being optionally substituted

Preferably, R⁹ is selected in the group consisting of optionally substituted (C₁-C₁₂)alkyl, optionally substituted (C₂-C₁₂) alkenyl, optionally substituted (C₂-C₁₂) alkynyl, and aryl optionally substituted with a (C₁-C₆)alkyl; and
R¹⁰ to R¹⁴ are independently selected in the group consisting of optionally substituted (C₁-C₁₂)alkyl, optionally substituted (C₂-C₁₂) alkenyl, optionally substituted (C₂-C₁₂) alkynyl, optionally substituted aryl.

The invention also relates to a compound as defined above, or a pharmaceutically acceptable salt and/or solvate thereof, for use as a drug.

The invention also relates to a pharmaceutical composition comprising a compound as defined above or a pharmaceutically acceptable salt and/or solvate thereof, and at least one pharmaceutically acceptable excipient.

The invention also relates to a compound for use as defined above or a pharmaceutical composition as defined above, for use in preventing or treating iron- and/or copper-associated disorders, in particular iron and/or copper overload-associated disorders; or metal-dependent cell death associated disorders.

In a particular aspect, the iron- and/or copper-associated disorders are selected among HFE-related hematochromatosis; non HFE-related hematochromatosis; congenital atransferrinenemia; iron-loading associated anemias such as thalassemia, myelodysplasia and hematopoietic stem-cell transplantation-associated disorders; chronic liver diseases; chronic inflammation linked to cancer; autoimmune or inflammatory diseases; neurodegeneration with brain iron accumulation-associated diseases such as Wilson's Disease; and polygenic neurodegenerative-associated diseases such as Parkinson's Disease and Alzheimer Disease.

In a particular aspect, the metal-dependent cell death associated disorders are metalloptosis associated disorders, in particular disorders associated to ferroptosis and/or cuproptosis; preferably selected among Hemorrhagic stroke; Ischemic Heart Injury; Ischemic Renal Injury; Ischaemia-reperfusion injury (IRI); Neurodegenerative diseases selected from Huntington Disease, Dementia, amyotrophic lateral and sclerosis (ALS); aging related diseases or disorders; sepsis; ishemia-reperfusion; and Diabetes.

In a particular aspect, in the compounds of the invention, X is a (C₁-C₁₂)alkyl optionally substituted and/or optionally interrupted by an heteroatom, preferably a (C₁-C₅)alkyl optionally substituted and/or optionally interrupted by an heteroatom, more preferably X is an ethyl.

In a particular aspect, in the compounds of the invention, R⁵, R⁶, R⁷ and R⁸ are independently selected in the group consisting of H, halogen, optionally substituted (C₁-C₂₄)alkyl, optionally substituted (C₂-C₂₄) alkenyl, optionally substituted (C₂-C₂₄) alkynyl, preferably H, halogen, haloalkyl, or (C₁-C₂₄)alkyl.

In a particular aspect, in the compounds of the invention, R⁴ is H.

In a particular aspect, in the compounds of the invention, R¹ is H.

In a particular aspect, in the compounds of the invention, R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl, provided that at least one of R² and R³ is not H; or R² and R³ with the nitrogen atom to which they are bound form together an optionally substituted nitrogen-heterocycloalkyl comprising 3 to 10 ring atoms; R⁹ to R¹² being as defined above.

In a particular aspect, in the compounds of the invention, Y⁰, Y¹, Y² and Y³ are independently absent or -selected from the group consisting of NH—, —O—, —S—, —S(O)—, —S(O)₂—, —C(O)—, —C(O)—, —OC(O)—, —NHC(O)—, —C(O)NH—, —C(O)NR¹⁴—, (C₁-C₂₄)alkyl, —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴; the (C₁-C₂₄)alkyl groups being optionally substituted; and R¹³ and R¹⁴ being as defined above.

In a particular aspect, in the compounds of the invention, Y⁰, Y¹, Y² and Y³ are independently absent or —O—; —NH—, —S— or optionally substituted (C₁-C₂₄)alkyl; preferably Y⁰, Y¹, Y² and Y³ are independently absent or —O—; more preferably Y⁰ is —O— and Y¹, Y² and Y³ are independently absent or —O—.

In a particular aspect, the compound for use according to the invention is selected from compounds (001) to (064) and (A1) to (A4) of Table 1, or a pharmaceutically acceptable salt and/or solvate thereof.

In a particular aspect, the compound for use according to the invention is selected from compounds (001) to (038) and (A1) to (A4) of Table 1, or a pharmaceutically acceptable salt and/or solvate thereof.

In a particular aspect, the compound according to the invention is selected from compounds (001) to (064) of Table 1; preferably selected from compounds (002), (003), (004), (008), (009), (010), (012), (013), (014), (015), (016), (017), (018), (019), (020), (022), (023), (024), (025), (026), (028), (029), (036), (039), (040), (043), (045), (049), (051), (052), (054), (057), (058), (059), (061), (063), and (064) of Table 1; more preferably selected from compounds (003) to (007), (013), (014), (021), (022), (029) and (031) to (038) of Table 1; or a pharmaceutically acceptable salt and/or solvate thereof.

In a particular aspect, the compound according to the invention is selected from compounds (001) to (038) of Table 1; preferably selected from compounds (003) to (007), (013), (014), (021), (029) and (031) to (038) of Table 1; or a pharmaceutically acceptable salt and/or solvate thereof.

### BRIEF DESCRIPTION OF THE FIGURES

FIG. 1: Complexation studies of Serotonin, A3 and metals: Serotonin and A3 form differently colored solutions in the presence of FeCl3 and CuSO4, indicative of complex formation.

FIG. 2: NMR of Serotonin and derivatives with metals: Complexation studies of serotonin and derivatives show that these molecules interact with Fe2+, Fe3+ and Cu2+. Molecular structure of serotonin (FIG. 2A), ¹H NMR Spectrum 7.8 mM serotonin (HCl form), D₂O, 25° C. (FIG. 2B), 7.8 mM serotonin (HCl form), D₂O, Fe(NO₃)₃, 25° C. (FIG. 2C), 7.8 mM serotonin (HCl form), D₂O, FeCl₂, 25° C. (FIG. 2D); Molecular structure of derivatives A3 (FIG. 2E), ¹H NMR spectrum, 25 mM A3, MeOD, 25° C. (FIG. 2F), 25 mM A3, MeOD, Fe(NO₃)₃, 25° C. (FIG. 2G), 25 mM A3, MeOD, FeCl₂, 25° C. (FIG. 2H); Molecular structure of derivatives A3 (FIG. 2I), ¹H NMR spectrum 6.4 mM A3, MeOD, 37° C. (FIG. 2J), 6.4 mM A3, MeOD, Fe(NO3)3, 37° C. (FIG. 2K), 6.4 mM A3, MeOD, FeCl2, 37° C. (FIG. 2L); ¹H NMR spectrum, 6.4 mM A3, D₂O, 37° C. (FIG. 2M), 6.4 mM A3, D₂O, Fe(NO₃)₃, 37° C. (FIG. 2N), 7.7 mM A3 (basic form), D₂O, CuSO₄, 37° C. (FIG. 2O); Molecular structure of derivatives A3 (FIG. 2P), ¹H NMR spectrum, 20.2 mM A3, MeOD, 37° C. (FIG. 2Q), 20.2 mM A3, MeOD, FeCl₃, 37° C. (FIG. 2R); Molecular structure of derivatives LT265B (FIG. 2S), ¹H NMR spectrum, 7.8 mM LT265B, D₂O, 37° C. (FIG. 2T), 7.8 mM LT265B, D₂O, FeCl₃, 37° C. (FIG. 2U); Molecular structure of derivatives LT262A (FIG. 2V), ¹H NMR spectrum, 6 mM LT262A, D₂O, 37° C. (FIG. 2W), 6 mM LT262A, D₂O, FeCl₃, 37° C. (FIG. 2X); Molecular structure of derivatives LC336 (FIG. 2Y), ¹H NMR spectrum, 15.3 mM LC336, D₂O, 37° C. (FIG. 2Z), 15.3 mM LC336, D₂O, FeCl₃, 37° C. (FIG. 2AA), Molecular structure of derivatives LC357 (FIG. 2AB), ¹H NMR spectrum, 6.2 mM LC357, D₂O, 37° C. (FIG. 2AC), 6.2 mM LC357, D₂O, FeCl₃, 37° C. (FIG. 2AD).

FIG. 3: Serotonin analog localizes in lysosomes: HMLER cells were treated with 1 μM A3 for 3 h and with LysoTracker for 45 mins. Cells were then washed, fixed and permeabilized. Subsequently, using Alexa-488-N₃ and click chemistry, A3 was visualized and found to localize in lysosomes.

FIG. 4: Serotonin analogs increase lysosomal iron content: a) Serotonin analogs used in this figure (FIG. 4a). b) Flow cytometry analysis of RhoNox-M fluorescence in BELA cells treated with respective molecules (10 μM) for 24 h. RhoNox-M is a lysosome-specific probe for Fe²⁺ (FIG. 4b).

FIG. 5: Serotonin analogs increase total cellular iron and copper content: a) Inductively coupled plasma mass spectrometry (ICP-MS) analysis of total cellular iron in HMLER cells treated with 100 nM A3 (FIG. 5a). b) ICP-MS analysis of total cellular copper in HMLER cells treated with 100 nM A3 (FIG. 5b).

FIG. 6: Serotonin analogs decrease lipid peroxidation caused by ferroptosis inducers: a) Cells treated with a ferroptosis inducer and co-treated with A3 or Liproxstatin-1 (FIG. 6a). A3 reduces lipid peroxidation caused by a ferroptosis inducer. b) A3 rescues cell death caused by a ferroptosis inducer (FIG. 6b).

FIG. 7: Serotonin analogs rescue cell death caused by the ferroptosis inducer RSL3: Quantification of flow cytometry profiles of Annexin/propidium iodide of cells treated with various concentrations of RSL3 and co-treated with A3 or Liproxstatiin-1, showing that A3 protects against RSL3-induced ferroptosis (FIG. 7b).

FIG. 8: Normalised mean fluorescence intensity fold change of BODIPY-C11 obtained from flow cytometry analysis of PDAC cells co-treated with ferroptosis inducer RSL3 (200 nM) and serotonin derivatives (10 μM).

FIG. 9: a. Diagram of click labelling of cells; PDAC cells were treated with 10 μM A3 for 3 h and (b,h) with LysoTracker for 45 mins. Cells were then washed, fixed and permeabilized. Subsequently, using Alexa-488-N3 and click chemistry, A3 was labelled and the corresponding antibodies for cell organelles were labelled for c-g. A3 was found to localize in lysosomes and not on other cell organelles.

FIG. 10: a. Quantification of flow cytometry profiles of Annexin/propidium iodide of cells treated with various concentrations of RSL3 and co-treated with A3, A0 or Liproxstatiin-1. A3 protects against RSL3-induced ferroptosis. b. Cyclic voltammetry curve of FeCl₃ and after addition of compounds as indicated.

### DETAILED DESCRIPTION OF THE INVENTION

### Definitions

For the purpose of the invention, the term “pharmaceutically acceptable” is intended to mean what is useful to the preparation of a pharmaceutical composition, and what is generally safe and non-toxic, for a pharmaceutical use.

The term “pharmaceutically acceptable salt” is intended to mean, in the framework of the present invention, a salt of a compound which is pharmaceutically acceptable, as defined above, and which possesses the pharmacological activity of the corresponding compound.

The pharmaceutically acceptable salts comprise:
(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and (2) salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine. N-methylglucamine, triethanolamine, tromethamine and the like. Acceptable inorganic bases comprise aluminium hydroxide, calcium hydroxide, potassium hydroxide, sodium carbonate and sodium hydroxide.

The “stereoisomers” are isomeric compounds that have the same molecular formula and sequence of bonded atoms, but differ in the 3D-dimensional orientations of their atoms in space. The stereoisomers include enantiomers, diastereoisomers, Cis-trans and E-Z isomers, conformers, and anomers. In a specific aspect of the disclosure, the stereoisomers include diastereoisomers and enantiomers.

The “tautomers” are isomeric compounds that differ only in the position of the protons and the electrons.

The “solvates” of the present disclosure include conventional solvates such as those formed during the last step of the preparation of the compounds of the invention due to the presence of solvents. It can be for example an hydrate or an alcoholate such as an ethanolate.

The term “halogen” or “halo”, as used in the invention, refers to a fluorine, bromine, chlorine or iodine atom.

The term “Cₓ-C_{y}” in which x and y are integers, as used in the present disclosure, means that the corresponding hydrocarbon chain comprises from x to y carbon atoms. If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from 1 to 6 carbon atoms, especially 1, 2, 3, 4, 5 or 6 carbon atoms. The term “C₀” thus means that no hydrocarbon chain is present but only a single bond.

The term “alkyl”, as used in the invention, refers to a monovalent linear or branched saturated hydrocarbon chain. For example, the term “(C₁-C₃)alkyl” more specifically means methyl, ethyl, n-propyl, or isopropyl. The term “(C₁-C₆)alkyl” more specifically means methyl, ethyl, n-propyl, isopropyl, n-butyl, iso-butyl, sec-butyl, tert-butyl, pentyl or linear or branched hexyl.

The term «heteroalkyl» refers to an alkyl as defined herein, wherein the aliphatic carbon chain comprises at one or both of its two ends (in particular, the end attached to the remainder of the molecule), and/or is interrupted by at least one heteroatom such as O, N or S. Examples of heteroalkyl are in particular alkoxy (—O-alkyl), alkylthio (—S-alkyl), and alkylamino (—NH(alkyl) or —N(alkyl)₂). A «C₁-C₆ heteroalkyl» refers to a heteroalkyl having 1 to 6 carbon atoms and at least one heteroatom such as O, N or S. Examples of heteroalkyl (or C₁-C₆ heteroalkyl) include, but are not limited to, methoxy, ethoxy, propyloxy, isopropyloxy, butyloxy, isobutyloxy, tert-butyloxy, pentyloxy, hexyloxy, methylthio, ethylthio, propylthio, isopropylthio, butylthio, isobutylthio, tert-butylthio, pentylthio, hexylthio, methylamino, ethylamino, propylamino, isopropylamino, butylamino, isobutylamino, tert-butylamino, pentylamino, or hexylamino.

The term “alkoxy” or “alkyloxy”, as used in the invention, refers to an alkyl group as defined above bound to the molecule via an oxygen atom. (C₁-C₃) alkoxy includes methoxy, ethoxy, propyloxy, and isopropyloxy. (C₁-C₅) alkoxy includes methoxy, ethoxy, propyloxy, isopropyloxy, butyloxy, isobutyloxy, tert-butyloxy, pentyloxy and hexyloxy. In a particular embodiment, the “alkoxy” or “alkyloxy” is a methoxy.

The term “haloalkyl” refers to an alkyl as defined herein substituted with at least one halogen, for example one, two or three halogens. A “(C₁-C₆)haloalkyl” refers to a haloalkyl having 1 to 6 carbon atoms and at least one halogen such as one fluorine, chlorine, bromine, or iodine atom. The haloalkyl includes a fluoromethyl (—CH₂F), a difluoromethyl (—CHF₂), or a trifluoromethyl (—CF₃).

The term “alkenyl”, as used in the invention, refers to a straight or branched monovalent unsaturated hydrocarbon chain comprising at least one double bond including, but not limited to, ethenyl, propenyl, butenyl, pentenyl, hexenyl and the like.

The term “alkynyl”, as used in the invention, refers to a straight or branched monovalent unsaturated hydrocarbon chain comprising at least one triple bond including, but not limited to, ethynyl, propynyl, butynyl, pentynyl, hexynyl and the like. Preferably, an alkynyl group as used in the present disclosure comprises one triple bond.

In particular, the term (C₀-C₆)alkyl-ethynyl, as used in the present disclosure, refers to an alkynyl as defined above comprising one terminal triple bond; i.e. a (C₀-C₆)alkyl terminally substituted by an ethynyl.

The term “cycloalkyl” corresponds to a saturated or unsaturated mono-, bi- or tri-cyclic alkyl group comprising between 3 and 20 atoms of carbons. It also includes fused, bridged, or spiro-connected cycloalkyl groups. The term “cycloalkyl” includes for instance cyclopropyl, cyclobutyl, cyclopentyl, and cyclohexyl, preferably cyclopropyl. The term “spirocycloalkyl” includes for instance a spirocyclopentyl. In a particular aspect, the term “cycloalkyl” corresponds to a saturated monocycloalkyl group comprising between 3 and 7 atoms of carbons. In a particular aspect, the cycloalkyl group is cyclohexyl.

The term “heterocycloalkyl” corresponds to a saturated or unsaturated cycloalkyl group as above defined further comprising at least one heteroatom such as nitrogen, oxygen, or sulphur atom. It also includes fused, bridged, or spiro-connected heterocycloalkyl groups. Representative heterocycloalkyl groups include, but are not limited to 3-dioxolane, benzo[1,3]dioxolyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranyl, tetrahydro-2H-pyranyl, tetrahydrofuranyl, and tetrahydrothiophenyl. The term “heterocycloalkyl” may also refer to a 5-10 membered bridged heterocyclyl such as 7-oxabicyclo[2,2,1]heptanyl.

The term “aryl” corresponds to a mono- or bi-cyclic aromatic hydrocarbons having from 6 to 12 carbon atoms. For instance, the term “aryl” includes phenyl, biphenyl, or naphthyl. In a particular embodiment, the aryl is a phenyl.

The term “heteroaryl” as used herein corresponds to an aromatic, mono- or poly-cyclic group comprising between 5 and 14 atoms, including at least one heteroatom such as nitrogen, oxygen or sulphur atom. Examples of such mono- and poly-cyclic heteroaryl group may be: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzisoxazolyl, oxindolyl, benzoxazolinyl, benzothienyl, benzothiazolyl, isatinyl, dihydropyridyl, pyrimidinyl, s-triazinyl, oxazolyl, or thiofuranyl. In a particular embodiment, the heteroaryl group is a thiophenyl, a pyridinyl, a pyrazinyl, or a thiazolyl.

The term “(C₁-C₆)alkyl-aryl”, as used herein, refers to a (C₁-C₆)alkyl group as defined above substituted with an aryl group as defined above. In particular, it can be a benzyl group.

The term “(C₁-C₆)alkyl-heterocycle”, as used herein, refers to a (C₁-C₆)alkyl group as defined above substituted with a heterocycle group as defined above. In particular, a “(C₁-C₆)alkyl-heteroaryl” refers to a (C₁-C₆)alkyl group as defined above substituted with a heteroaryl group as defined above.

The expression “substituted by at least” or “substituted by” means that the group is substituted by one or several substituents of the list. For instance, the expression “a C₁-C₆alkyl substituted by at least one halogen” or “a C₁-C₆alkyl substituted by a halogen” may include a fluoromethyl (—CH₂F), a difluoromethyl (—CHF₂), or a trifluoromethyl (—CF₃).

The expression “optionally substituted” means that the group is not substituted or is substituted by one or several substituents of the list.

In the context of the invention, the substituents can be selected in the group consisting of halogen; CN; NO₂; (C₁-C₆)alkyl; (C₁-C₆)haloalkyl; (C₁-C₆)haloalkoxy, (C₁-C₆)halothioalkoxy, optionally substituted aryl (in particular optionally substituted with one or two (C₁-C₆)alkyl or NO₂); heteroaryl; oxo; NR^{a}R^{b}; COR^{c}; CO₂R^{d}; CONR^{e}R^{f}; OR^{g}; SR^{h}; SO₂Rⁱ; SO₂NR^{j}R^{k}; NR^{l}COR^{m}; RⁿNR^{O}R^{P}; NR^{q}SO₂R^{r}; wherein R^{a} to R^{r} are, independently of one another, H, (C₁-C₆)alkyl, (C₁-C₆)haloalkyl, aryl, (C₁-C₆)alkyl-aryl, heteroaryl or (C₁-C₆)alkyl-heteroaryl.

By “—CO—” or “—C(O)—”, it refers to an oxo group. By “—SO—” or “—S(O)—”, it refers to a sulfinyl group.

By “—SO₂—” or “—S(O₂)—”, it refers to a sulfonyl group.

As used herein, the terms “subject”, “individual” or “patient” are interchangeable and refer to an animal, preferably to a mammal, even more preferably to a human, including adult and child. However, the term “subject” can also refer to non-human animals, in particular mammals such as dogs, cats, horses, cows, pigs, sheep and non-human primates, among others.

Within the context of the present disclosure, the term treatment denotes curative, symptomatic, and preventive treatment. Pharmaceutical compositions, kits, products and combined preparations of the invention can be used in humans with a disease or disorder. The pharmaceutical compositions, kits, products and combined preparations of the invention will not necessarily cure the patient but will delay or slow the progression or prevent further progression of the disease or disorder, and/or ameliorating thereby the patients' condition. In treating the disease or disorder, the pharmaceutical composition of the invention is administered in a therapeutically effective amount.

Whenever within this whole specification “treatment of a disease or disorder” or the like is mentioned with reference to the pharmaceutical composition of the invention, there is meant: a) a method for treating a disease or disorder, said method comprising administering a therapeutically effective amount of a compound of the invention or of a pharmaceutical composition comprising said compound to a subject in need of such treatment; b) the use of a compound of the invention or of a pharmaceutical composition comprising said compound for the treatment of a disease or disorder; c) the use of a compound of the invention or of a pharmaceutical composition comprising said compound for the manufacture of a medicament for the treatment of a disease or disorder; and/or d) a compound of the invention or of a pharmaceutical composition comprising said compound for use in the treatment a disease or disorder.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

By “therapeutically effective amount” or “active amount” or “effective amount”, it is meant the quantity of the pharmaceutical composition of the invention which prevents, removes or reduces the deleterious effects of a disease or disorder in mammals, including humans, alone or in combination with the other active ingredients of the pharmaceutical composition, kit, product or combined preparation. It is understood that the administered dose may be lower for each compound in the composition to the “therapeutic effective amount” define for each compound used alone or in combination with other treatments than the combination described here. The “therapeutic effective amount” of the composition will be adapted by those skilled in the art according to the patient, the pathology, the mode of administration, etc.

As used herein, the term “pharmaceutically acceptable excipient” refers to any ingredient except active ingredients which are present in a pharmaceutical composition. Its addition may be aimed to confer a particular consistency or other physical or gustative properties to the final product. A pharmaceutically acceptable excipient must be devoid of any interaction, in particular chemical, with the active ingredients.

In the context of the invention, the ranges of values expressed by “from X to XX” or “between X and XX” comprise the upper and lower limits.

### Compounds for Use

As mentioned, the invention relates to a compound of the following general formula (I), or a pharmaceutically acceptable salt, stereoisomer, tautomer or solvate thereof, for use for preventing and/or treating metalloptosis associated disorders, or a pharmaceutically acceptable salt and/or solvate thereof, wherein formula (I) is:

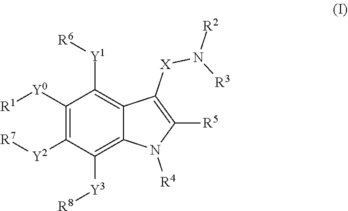

wherein
R¹, R², R³ and R⁴ are independently selected in the group consisting of H, OH, —C(O)OR⁹—, —S(O)₂R¹⁰—, —C(O)R⁹—, —S(O)R¹⁰—, —NR¹¹R¹², optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl, provided that at least one of R¹, R² and R³ is not H;
or
R¹ and R⁴ are independently selected in the group consisting of H, OH, —C(O)OR⁹—, —S(O)₂R¹⁰—, —C(O)R⁹—, —S(O)R¹⁰—, —NR¹¹R¹²—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl, and R² and R³ form together with the nitrogen atom to which they bound an optionally substituted heterocycloalkyl; X is selected in the group consisting of (C₁-C₁₂)alkyl, —O—(C₁-C₁₂)alkyl-, —C(O)—, —C(O)—(C₁-C₁₂)alkyl- and —NH—C(O)—(C₁-C₁₂)alkyl-, —S—(C₁-C₁₂)alkyl-, —S(O)—(C₁-C₁₂)alkyl-, —S(O)₂—(C₁-C₁₂)alkyl-; the (C₁-C₁₂)alkyl groups being optionally substituted and/or optionally interrupted by an heteroatom;
Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴—, (C₁-C₂₄)alkyl, —NH—C(O)—NH—, —NH—S(O)—NH—, —NH—S(O₂)—NH—, —S—C(O)—NH—, —O—C(O)—NH—, —O—S(O)—NH—, —O—S(O₂)—NH—, —S—CH₂—C(O)—NH—, —S—CH₂—S(O)—NH—, —S—CH₂—S(O₂)—NH—, —S(O)—CH₂—C(O)—NH—, —S(O₂)—CH₂—C(O)—NH—, —O—CH₂—C(O)—NH—, —O—CH₂—S(O)—NH—, —O—CH₂—S(O₂)—NH—, —NH—CH₂—C(O)—NH—, —NH—CH₂—S(O)—NH—, —NH—CH₂—S(O₂)—NH—, —O—(CH₂)₂—NH—C(O)—, —O—(CH₂)₂—N(CH₃)—C(O)—, —O—(CH₂)₂—NH—S(O)—, —O—(CH₂)₂—N(CH₃)—S(O)—, —O—(CH₂)₂—NH—S(O₂)—, —O—(CH₂)₂—N(CH₃)—S(O₂)—, —S—(CH₂)₂—NH—C(O)—, —S—(CH₂)₂—N(CH₃)—C(O)—, —S—(CH₂)₂—NH—S(O)—, —S—(CH₂)₂—N(CH₃)—S(O)—, —S—(CH₂)₂—NH—S(O₂)—, —S—(CH₂)₂—N(CH₃)—S(O₂)—, —NH—(CH₂)₂—NH—C(O)—, —NH—(CH₂)₂—N(CH₃)—C(O)—, —NH—(CH₂)₂—NH—S(O)—, —NH—(CH₂)₂—N(CH₃)—S(O)—, —NH—(CH₂)₂—NH—S(O₂)—, —NH—(CH₂)₂—N(CH₃)—S(O₂)—, —O—CH₂—CH(CH₂—OH)—NH—C(O)—, —O—CH₂—CH(CH₂—OCH₃)—NH—C(O)—, —O—CH₂—CH(CH₃)—NH—C(O)—, —O—CH(CH₃)—CH₂—NH—C(O)—, —O—CH₂—CH(CH₂—OH)—NH—S(O)—, —O—CH₂—CH(CH₂—OCH₃)—NH—S(O)—, —O—CH₂—CH(CH₃)—NH—S(O)—, —O—CH(CH₃)—CH₂—NH—S(O)—, —O—CH₂—CH(CH₂—OH)—NH—S(O₂)—, —O—CH₂—CH(CH₂—OCH₃)—NH—S(O₂)—, —O—CH₂—CH(CH₃)—NH—S(O₂)—, —O—CH(CH₃)—CH₂—NH—S(O₂)—, —S—CH₂—CH(CH₂—OH)—NH—C(O)—, —S—CH₂—CH(CH₂—OCH₃)—NH—C(O)—, —S—CH₂—CH(CH₃)—NH—C(O)—, —S—CH(CH₃)—CH₂—NH—C(O)—, —S—CH₂—CH(CH₂—OH)—NH—S(O)—, —S—CH₂—CH(CH₂—OCH₃)—NH—S(O)—, —S—CH₂—CH(CH₃)—NH—S(O)—, —S—CH(CH₃)—CH₂—NH—S(O)—, —S—CH₂—CH(CH₂—OH)—NH—S(O₂)—, —S—CH₂—CH(CH₂—OCH₃)—NH—S(O₂)—, —S—CH₂—CH(CH₃)—NH—S(O₂)—, —S—CH(CH₃)—CH₂—NH—S(O₂)—, —NH—CH₂—CH(CH₂—OH)—NH—C(O)—, —NH—CH₂—CH(CH₂—OCH₃)—NH—C(O)—, —NH—CH₂—CH(CH₃)—NH—C(O)—, —NH—CH(CH₃)—CH₂—NH—C(O)—, —NH—CH₂—CH(CH₂—OH)—NH—S(O)—, —NH—CH₂—CH(CH₂—OCH₃)—NH—S(O)—, —NH—CH₂—CH(CH₃)—NH—S(O)—, —NH—CH(CH₃)—CH₂—NH—S(O)—, —NH—CH₂—CH(CH₂—OH)—NH—S(O₂)—, —NH—CH₂—CH(CH₂—OCH₃)—NH—S(O₂)—, —NH—CH₂—CH(CH₃)—NH—S(O₂)—, —NH—CH(CH₃)—CH₂—NH—S(O₂)—, —O—(CH₂)₂—NH—, —S—(CH₂)₂—NH—, and —NH—(CH₂)₂—NH—; the (C₁-C₂₄)alkyl groups being optionally substituted; and
R⁵, R⁶, R⁷ and R⁸ are independently selected in the group consisting of H, halogen, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl; and
R⁹ to R¹⁴ are independently selected in the group consisting of optionally substituted (C₁-C₁₂)alkyl, optionally substituted (C₂-C₁₂) alkenyl, optionally substituted (C₂-C₁₂) alkynyl, optionally substituted aryl.

In particular, R⁹ is selected in the group consisting of optionally substituted (C₁-C₁₂)alkyl, optionally substituted (C₂-C₁₂) alkenyl, optionally substituted (C₂-C₁₂) alkynyl, and aryl optionally substituted with a (C₁-C₆)alkyl; and
R¹⁰ to R¹⁴ are independently selected in the group consisting of optionally substituted (C₁-C₁₂)alkyl, optionally substituted (C₂-C₁₂) alkenyl, optionally substituted (C₂-C₁₂) alkynyl, and optionally substituted aryl.

The invention also relates to the use of a compound of general formula (I) as defined above, especially one of those as disclosed in Table 1, or a pharmaceutically acceptable salt and/or solvate thereof, or a pharmaceutical composition comprising it, for the manufacture of a medicament for preventing and/or treating metalloptosis associated disorders.

The invention further relates to a method for preventing and/or treating metalloptosis associated disorders in a subject in need thereof, comprising administering a therapeutically effective amount of a compound of general formula (I) as defined above, especially one of those as disclosed in Table 1, or a pharmaceutically acceptable salt and/or solvate thereof, or a pharmaceutical composition comprising it, to said subject.

In a specific embodiment, the metalloptosis associated disorders are disorders associated to ferroptosis and/or cuproptosis. Advantageously, the metalloptosis associated disorders are selected among Hemorrhagic stroke; Ischemic Heart Injury; Ischemic Renal Injury; Ischaemia-reperfusion injury (IRI) such as IRI occurring during organ transplantation ((liver, lung, heart, kidneys, pancreas, intestine); Neurodegenerative diseases selected from Huntington Disease, Dementia, and amyotrophic lateral sclerosis (ALS); aging related diseases or disorders; sepsis; ishemia-reperfusion; and Diabetes. In particular, the use of the compound according to the invention is specifically disclosed below in the specification.

In the context of the invention, the compound of general formula (I) as defined above, or a pharmaceutically acceptable salt and/or solvate thereof, can also be used for preventing and/or treating iron- and/or copper-associated disorders. The invention can also relate to the use of a compound of general formula (I) as defined above, or a pharmaceutically acceptable salt and/or solvate thereof, or a pharmaceutical composition comprising it, for the manufacture of a medicine for use for preventing and/or treating iron- and/or copper-associated disorders. The invention can also relates to a method for preventing and/or treating iron- and/or copper-associated disorders in a subject in need thereof, comprising administering a therapeutically effective amount of a compound of general formula (I) as defined above, or a pharmaceutically acceptable salt and/or solvate thereof, or a pharmaceutical composition comprising it to said subject.

In this context, the iron- and/or copper-associated disorders can be selected among HFE-related hematochromatosis; non HFE-related hematochromatosis; congenital atransferrinenemia; iron-loading associated anemias such as thalassemia, myelodysplasia and hematopoietic stem-cell transplantation-associated disorders; chronic liver diseases; chronic inflammation linked to cancer; autoimmune or inflammatory diseases; neurodegeneration with brain copper- or iron accumulation-associated diseases such as Wilson's Disease; and polygenic neurodegenerative-associated diseases such as Parkinson's Disease and Alzheimer Disease.

In particular, the iron-associated disorders can be selected among HFE-related hematochromatosis; non HFE-related hematochromatosis; congenital atransferrinenemia; iron-loading associated anemias such as thalassemia, myelodysplasia and hematopoietic stem-cell transplantation-associated disorders; chronic liver diseases; chronic inflammation linked to cancer; autoimmune or inflammatory diseases; neurodegeneration with brain iron accumulation-associated diseases such as Wilson's Disease; and polygenic neurodegenerative-associated diseases such as Parkinson's Disease and Alzheimer Disease.

In particular, the copper-associated disorders can be selected among HFE-related hematochromatosis; non HFE-related hematochromatosis; congenital atransferrinenemia; iron-loading associated anemias such as thalassemia, myelodysplasia and hematopoietic stem-cell transplantation-associated disorders; chronic liver diseases; chronic inflammation linked to cancer; autoimmune or inflammatory diseases; neurodegeneration with brain copper accumulation-associated diseases such as Wilson's Disease; and polygenic neurodegenerative-associated diseases such as Parkinson's Disease and Alzheimer Disease.

In the compound of general formula (I), X is selected in the group consisting of (C₁-C₁₂)alkyl, —O—(C₁-C₁₂)alkyl-, —C(O)—, —C(O)—(C₁-C₁₂)alkyl-, —NH—C(O)—(C₁-C₁₂)alkyl-, —S—(C₁-C₁₂)alkyl-, —S(O)—(C₁-C₁₂)alkyl-, and —S(O)₂—(C₁-C₁₂)alkyl-; the (C₁-C₁₂)alkyl groups being optionally substituted and/or optionally interrupted by an heteroatom. Preferably X is selected in the group consisting of (C₁-C₁₂)alkyl, —O—(C₁-C₁₂)alkyl-, —C(O)—, —C(O)—(C₁-C₁₂)alkyl-, —NH—C(O)—(C₁-C₁₂)alkyl-; the (C₁-C₁₂)alkyl groups being optionally substituted and/or optionally interrupted by an heteroatom. Preferably, X is selected in the group consisting of (C₁-C₆)alkyl, —O—(C₁-C₅)alkyl-, —C(O)—, —C(O)—(C₁-C₆)alkyl- and —NH—C(O)—(C₁-C₆)alkyl-; the (C₁-C₆)alkyl groups being optionally substituted and/or optionally interrupted by an heteroatom. More preferably, X is selected in the group consisting of (C₁-C₃)alkyl, —O—(C₁-C₃)alkyl-, —C(O)—(C₁-C₃)alkyl- and —NH—C(O)—(C₁-C₃)alkyl-; the (C₁-C₃)alkyl groups being optionally substituted and/or optionally interrupted by an heteroatom.

In a specific embodiment, X is a (C₁-C₆)alkyl. Preferably X is a methyl, an ethyl or a n-propyl, more preferably an ethyl.

In the compound of general formula (I), Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴, optionally substituted (C₁-C₂₄)alkyl, —NH—C(O)—NH—, —NH—S(O)—NH—, —NH—S(O₂)—NH—, —S—C(O)—NH—, —O—C(O)—NH—, —O—S(O)—NH—, —O—S(O₂)—NH—, —S—CH₂—C(O)—NH—, —S—CH₂—S(O)—NH—, —S—CH₂—S(O₂)—NH—, —S(O)—CH₂—C(O)—NH—, —S(O₂)—CH₂—C(O)—NH—, —O—CH₂—C(O)—NH—, —O—CH₂—S(O)—NH—, —O—CH₂—S(O₂)—NH—, —NH—CH₂—C(O)—NH—, —NH—CH₂—S(O)—NH—, —NH—CH₂—S(O₂)—NH—, —O—(CH₂)₂—NH—C(O)—, —O—(CH₂)₂—N(CH₃)—C(O)—, —O—(CH₂)₂—NH—S(O)—, —O—(CH₂)₂—N(CH₃)—S(O)—, —O—(CH₂)₂—NH—S(O₂)—, —O—(CH₂)₂—N(CH₃)—S(O₂)—, —S—(CH₂)₂—NH—C(O)—, —S—(CH₂)₂—N(CH₃)—C(O)—, —S—(CH₂)₂—NH—S(O)—, —S—(CH₂)₂—N(CH₃)—S(O)—, —S—(CH₂)₂—NH—S(O₂)—, —S—(CH₂)₂—N(CH₃)—S(O₂)—, —NH—(CH₂)₂—NH—C(O)—, —NH—(CH₂)₂—N(CH₃)—C(O)—, —NH—(CH₂)₂—NH—S(O)—, —NH—(CH₂)₂—N(CH₃)—S(O)—, —NH—(CH₂)₂—NH—S(O₂)—, —NH—(CH₂)₂—N(CH₃)—S(O₂)—, —O—CH₂—CH(CH₂—OH)—NH—C(O)—, —O—CH₂—CH(CH₂—OCH₃)—NH—C(O)—, —O—CH₂—CH(CH₃)—NH—C(O)—, —O—CH(CH₃)—CH₂—NH—C(O)—, —O—CH₂—CH(CH₂—OH)—NH—S(O)—, —O—CH₂—CH(CH₂—OCH₃)—NH—S(O)—, —O—CH₂—CH(CH₃)—NH—S(O)—, —O—CH(CH₃)—CH₂—NH—S(O)—, —O—CH₂—CH(CH₂—OH)—NH—S(O₂)—, —O—CH₂—CH(CH₂—OCH₃)—NH—S(O₂)—, —O—CH₂—CH(CH₃)—NH—S(O₂)—, —O—CH(CH₃)—CH₂—NH—S(O₂)—, —S—CH₂—CH(CH₂—OH)—NH—C(O)—, —S—CH₂—CH(CH₂—OCH₃)—NH—C(O)—, —S—CH₂—CH(CH₃)—NH—C(O)—, —S—CH(CH₃)—CH₂—NH—C(O)—, —S—CH₂—CH(CH₂—OH)—NH—S(O)—, —S—CH₂—CH(CH₂—OCH₃)—NH—S(O)—, —S—CH₂—CH(CH₃)—NH—S(O)—, —S—CH(CH₃)—CH₂—NH—S(O)—, —S—CH₂—CH(CH₂—OH)—NH—S(O₂)—, —S—CH₂—CH(CH₂—OCH₃)—NH—S(O₂)—, —S—CH₂—CH(CH₃)—NH—S(O₂)—, —S—CH(CH₃)—CH₂—NH—S(O₂)—, —NH—CH₂—CH(CH₂—OH)—NH—C(O)—, —NH—CH₂—CH(CH₂—OCH₃)—NH—C(O)—, —NH—CH₂—CH(CH₃)—NH—C(O)—, —NH—CH(CH₃)—CH₂—NH—C(O)—, —NH—CH₂—CH(CH₂—OH)—NH—S(O)—, —NH—CH₂—CH(CH₂—OCH₃)—NH—S(O)—, —NH—CH₂—CH(CH₃)—NH—S(O)—, —NH—CH(CH₃)—CH₂—NH—S(O)—, —NH—CH₂—CH(CH₂—OH)—NH—S(O₂)—, —NH—CH₂—CH(CH₂—OCH₃)—NH—S(O₂)—, —NH—CH₂—CH(CH₃)—NH—S(O₂)—, —NH—CH(CH₃)—CH₂—NH—S(O₂)—, —O—(CH₂)₂—NH—, —S—(CH₂)₂—NH—, and —NH—(CH₂)₂—NH—; the (C₁-C₂₄)alkyl groups being optionally substituted.

In a specific embodiment, in the compound of general formula (I), Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴, optionally substituted (C₁-C₂₄)alkyl, —NH—C(O)—NH—, —NH—S(O)—NH—, —NH—S(O₂)—NH—, —S—C(O)—NH—, —O—C(O)—NH—, —O—S(O)—NH—, —O—S(O₂)—NH—, —O—(CH₂)₂—NH—, —S—(CH₂)₂—NH—, and —NH—(CH₂)₂—NH—; the (C₁-C₂₄)alkyl groups being optionally substituted.

In a particular embodiment, in the compound of general formula (I), Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴—, and optionally substituted (C₁-C₂₄)alkyl; the (C₁-C₂₄)alkyl groups being optionally substituted. In particular, Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₁₂)alkyl-NH—, —(C₁-C₁₂)alkyl-O—, —(C₁-C₁₂)alkyl-S—, (C₁-C₁₂)alkyl-NR¹³—, —(C₁-C₁₂)alkyl-S(O)—, —(C₁-C₁₂)alkyl-S(O)₂—, —(C₁-C₁₂)alkyl-C(O)—, —(C₁-C₁₂)alkyl-C(O)O—, —(C₁-C₁₂)alkyl-OC(O)—, —(C₁-C₁₂)alkyl-NHC(O)—, —(C₁-C₁₂)alkyl-C(O)NH—, —(C₁-C₁₂)alkyl-C(O)NR¹⁴—, and optionally substituted (C₁-C₁₂)alkyl; the (C₁-C₁₂)alkyl groups being optionally substituted. More particularly, Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₆)alkyl-NH—, —(C₁-C₆)alkyl-O—, —(C₁-C₆)alkyl-S—, (C₁-C₆)alkyl-NR¹³—, —(C₁-C₆)alkyl-S(O)—, —(C₁-C₅)alkyl-S(O)₂—, —(C₁-C₆)alkyl-C(O)—, —(C₁-C₅)alkyl-C(O)O—, —(C₁-C₆)alkyl-OC(O)—, —(C₁-C₆)alkyl-NHC(O)—, —(C₁-C₆)alkyl-C(O)NH—, —(C₁-C₆)alkyl-C(O)NR¹⁴—, and optionally substituted (C₁-C₆)alkyl; the (C₁-C₆)alkyl groups being optionally substituted. Preferably, Y⁰, Y¹, Y² and Y³ are independently absent or selected from —O—, and optionally substituted (C₁-C₆)alkyl.

In a specific embodiment, Y⁰, Y¹, Y² and Y³ are independently absent or —O—. Preferably Y⁰ is —O— and, Y¹, Y² and Y³ are independently absent or —O—, preferably absent.

In a specific embodiment, in the compound of general formula (I), X is a (C₁-C₅)alkyl, Y⁰ is —O—, and Y¹, Y² and Y³ are independently absent or —O—.

In a particular embodiment, X is a methyl, an ethyl or a n-propyl, Y⁰ is —O—, and Y¹, Y² and Y³ are independently absent or —O—. Preferably, X is a methyl, an ethyl or a n-propyl, Y⁰ is —O—, and Y¹, Y² and Y³ are absent. More preferably, X is an ethyl, Y⁰ is —O—, and Y¹, Y² and Y³ are absent.

In the compound of general formula (I), R⁵, R⁶, R⁷ and R⁸ independently selected in the group consisting of H, halogen, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl.

In a specific embodiment, R⁵, R⁶, R⁷ and R⁸ are independently selected in the group consisting of H, halogen, optionally substituted (C₁-C₂₄)alkyl, optionally substituted (C₂-C₂₄) alkenyl, optionally substituted (C₂-C₂₄) alkynyl. In particular, R₅, R₆, R₇ and R⁸ are independently selected in the group consisting of H, halogen, optionally substituted (C₁-C₁₂)alkyl, optionally substituted (C₂-C₁₂) alkenyl, optionally substituted (C₂-C₁₂) alkynyl. More particularly, R⁵, R⁶, R⁷ and R⁸ are independently selected in the group consisting of H, halogen, optionally substituted (C₁-C₆)alkyl, optionally substituted (C₂-C₆) alkenyl, optionally substituted (C₂-C₆) alkynyl. Preferably R⁵, R⁶, R⁷ and R⁸ are independently selected in the group consisting of H, halogen, haloalkyl, or (C₁-C₂₄)alkyl. More preferably R⁵, R⁶, R⁷ and R⁸ are independently selected in the group consisting of H, halogen, haloalkyl such as CF₃, or (C₁-C₅)alkyl.

In a specific embodiment, three of R⁵, R⁶, R⁷ and R⁸ are H, and the last one is as defined above.

In another specific embodiment, two of R⁵, R⁶, R⁷ and R⁸ are H, and two are as defined above.

In another specific embodiment, three of R⁵, R⁶, R⁷ and R⁸ are H, and one is as defined above.

In another specific embodiment, R⁵, R⁶, R⁷ and R⁸ are H.

In another specific embodiment, R⁵, R⁶, R⁷ and R⁸ are as defined above but not H.

In a particular embodiment, one or two of R⁵, R⁶, R⁷ and R⁸ are selected in the group consisting of H, halogen, haloalkyl such as CF₃, or (C₁-C₆)alkyl, and the others are H.

In a specific embodiment, Y⁰ is —O—, and Y¹, Y² and Y³ are absent, and:
three of R⁵, R⁶, R⁷ and R⁸ are H, and the last one is as defined above; or
two of R⁵, R⁶, R⁷ and R⁸ are H, and two are as defined above; or
three of R⁵, R⁶, R⁷ and R⁸ are H, and one is as defined above; or
R⁵, R⁶, R⁷ and R⁸ are H; or
R⁵, R⁶, R⁷ and R⁸ are as defined above but not H.

In a particular embodiment, Y⁰ is —O—, and Y¹, Y² and Y³ are absent, one or two of R⁵, R⁶, R⁷ and R⁸ are selected in the group consisting of H, halogen, haloalkyl such as CF₃, or (C₁-C₆)alkyl, and the others are H.

In one embodiment of the compound of general formula (I), R¹, R², R³ and R⁴ are independently selected in the group consisting of H, OH, —C(O)OR⁹—, —S(O)₂R¹⁰—, —C(O)R⁹—, —S(O)R¹⁰—, —NR¹¹R¹²—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl, optionally substituted (C₁-C₆)alkyl-aryl, and optionally substituted heteroaryl, provided that at least one of R¹, R² and R³ is not H.

In a particular embodiment of the compound of general formula (I), R¹, R², R³ and R⁴ are independently selected in the group consisting of H, OH, —C(O)OR⁹—, —S(O)₂R¹⁰—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl, optionally substituted (C₁-C₆)alkyl-aryl, and optionally substituted heteroaryl, provided that at least one of R¹, R² and R³ is not H.

In this definition, “optionally substituted” preferably means non-substituted or substituted with at least one, for example 1, 2 or 3, substituent(s) which may be selected in the group consisting of halogen; CN; NO₂; (C₁-C₆)alkyl; (C₁-C₆)haloalkyl; (C₁-C₆)haloalkoxy, (C₁-C₅)halothioalkoxy, optionally substituted aryl (in particular optionally substituted with one or two (C₁-C₆)alkyl or NO₂); heteroaryl; halogeno-heteroaryl, in particular halogeno-furanyl; oxo; NR^{a}R^{b}; COR^{c}; CO₂R^{d}; CONR^{e}R^{f}; OR^{g}; SR^{h}; SO₂Rⁱ; SO₂NR^{j}R^{k}; NR^{l}COR^{m}; RⁿNR^{O}R^{P}; NR^{q}SO₂R^{r}; wherein R^{a} to R^{b} are, independently of one another, H, (C₁-C₆)alkyl, (C₁-C₅)haloalkyl, aryl, (C₁-C₅)alkyl-aryl, heteroaryl or (C₁-C₆)alkyl-heteroaryl; in particular consisting of halogen; CN; NO₂; (C₁-C₆)alkyl; (C₁-C₆)haloalkyl; aryl optionally substituted with one or two (C₁-C₆)alkyl or NO₂; heteroaryl; oxo; NR^{a}R^{b}; COR^{c}; CO₂R^{d}; CONR^{e}R^{f}; and OR^{g}; wherein R^{a} to R^{g} are, independently of one another, H, (C₁-C₆)alkyl, (C₁-C₆)haloalkyl, aryl, (C₁-C₆)alkyl-aryl, heteroaryl or (C₁-C₆)alkyl-heteroaryl.

In a specific embodiment, R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH. In particular, R¹ is selected in the group consisting of H, (C₁-C₁₂)alkyl, (C₂-C₁₂) alkenyl, (C₂-C₁₂) alkynyl and aryl. Preferably, R¹ is H.

In a specific embodiment, R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH. In particular, R⁴ is selected in the group consisting of H, (C₁-C₁₂)alkyl and aryl. Preferably, R⁴ is H.

In a specific embodiment, R² and R³ are independently selected in the group consisting of H, OH, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl, provided that at least one of R¹, R² and R³ is not H, preferably at least one of R² and R³ is not H; or R² and R³ form together with the nitrogen atom to which they bound an optionally substituted heterocycloalkyl. In this definition, “optionally substituted” preferably means non-substituted or substituted with at least one, for example 1, 2 or 3, substituent(s) which may be preferably a halogen; CN; NO₂; (C₁-C₆)alkyl; (C₁-C₆)haloalkyl; (C₁-C₅)haloalkoxy, (C₁-C₆)halothioalkoxy, optionally substituted aryl (in particular optionally substituted with one or two (C₁-C₆)alkyl or NO₂; heteroaryl; halogeno-heteroaryl in particular halogeno-furanyl; oxo; NR^{a}R^{b}; COR^{c}; CO₂R^{d}; CONR^{e}R^{f}; OR^{g}; SR^{h}; SO₂Rⁱ; SO₂NR^{j}R^{k}; NR^{l}COR^{m}; RⁿNR^{O}R^{P}; NR^{q}SO₂R^{r}; wherein R^{a} to R^{r} are, independently of one another, H, (C₁-C₆)alkyl, (C₁-C₆)haloalkyl, aryl, (C₁-C₆)alkyl-aryl, heteroaryl or (C₁-C₆)alkyl-heteroaryl; in particular a (C₁-C₆)alkyl; halogen; CN; NO₂; (C₁-C₆)haloalkyl; phenyl optionally substituted with one or two (C₁-C₆)alkyl or NO₂; heteroaryl optionally substituted with —OH; oxo; NR^{a}R^{b}; (C₁-C₁₂)alkyl-NR^{a}R^{b}; COR^{c}; CO₂R^{d}; CONR^{e}R^{f}; and OR^{g}; wherein R^{a} to R^{b} are, independently of one another, H, (C₁-C₆)alkyl, (C₁-C₅)haloalkyl, aryl, (C₁-C₆)alkyl-aryl, heteroaryl or (C₁-C₆)alkyl-heteroaryl.

In the compound of general formula (I), when one or more of R¹, R², R³ and R⁴ is —C(O)OR⁹—, —S(O)₂R¹⁰—, —C(O)R⁹—, —S(O)R¹⁰—, or —NR¹¹R¹²—, preferably —C(O)OR⁹— or —S(O)₂R¹⁰—, R⁹ to R¹² are independently selected in the group consisting of optionally substituted (C₁-C₁₂)alkyl, optionally substituted (C₂-C₁₂) alkenyl, optionally substituted (C₂-C₁₂) alkynyl, and optionally substituted aryl.

In the compound of general formula (I), when one or more of R¹, R², R³ and R⁴ is —C(O)OR⁹—, —S(O)₂R¹⁰—, —C(O)R⁹—, —S(O)R¹⁰—, or —NR¹¹R¹²—, preferably —C(O)OR⁹— or —S(O)₂R¹⁰, R⁹ is selected in the group consisting of optionally substituted (C₁-C₁₂)alkyl, optionally substituted (C₂-C₁₂) alkenyl, optionally substituted (C₂-C₁₂) alkynyl, and aryl optionally substituted with a (C₁-C₆)alkyl; and R¹⁰ to R¹² are independently selected in the group consisting of optionally substituted (C₁-C₁₂)alkyl, optionally substituted (C₂-C₁₂) alkenyl, optionally substituted (C₂-C₁₂) alkynyl, and optionally substituted aryl.

In this definition, “optionally substituted” preferably means non-substituted or substituted with at least one, for example 1, 2 or 3, substituent(s) which may be preferably (C₁-C₆)alkyl.

In a specific embodiment, R⁹ to R¹² are independently selected in the group consisting of optionally substituted (C₁-C₆)alkyl, optionally substituted (C₂-C₆) alkenyl, optionally substituted (C₂-C₆) alkynyl, and optionally substituted aryl.

In particular, R⁹ is selected in the group consisting of optionally substituted (C₁-C₆)alkyl, optionally substituted (C₂-C₆) alkenyl, optionally substituted (C₂-C₆) alkynyl, and aryl optionally substituted with a (C₁-C₆)alkyl such as methyl; and R¹⁰ to R¹² are independently selected in the group consisting of optionally substituted (C₁-C₆)alkyl, optionally substituted (C₂-C₆) alkenyl, optionally substituted (C₂-C₆) alkynyl, and optionally substituted aryl.

Preferably, R⁹ to R¹² are independently selected in the group consisting of (C₁-C₆)alkyl, (C₂-C₆) alkenyl, (C₂-C₆) alkynyl, aryl or aryl substituted with a (C₁-C₆)alkyl such as methyl.

In another embodiment of the compound of general formula (I), R¹ and R⁴ are independently selected in the group consisting of H, OH, —C(O)OR⁹—, —S(O)₂R¹⁰—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl; and R² and R³ form together with the nitrogen atom to which they bound an optionally substituted heterocycloalkyl.

In a specific embodiment, the optionally substituted heterocycloalkyl formed by R² and R³ with the nitrogen atom to which they are bound form together is preferably an optionally substituted nitrogen-heterocycloalkyl comprising 3 to 10 ring atoms; preferably an optionally substituted nitrogen-heterocycloalkyl comprising one nitrogen atom and 3 to 10 ring atoms. In particular, the nitrogen-heterocycloalkyl is non substituted or substituted with one or more substituents selected in the list consisting of a halogen; CN; NO₂; (C₁-C₆)alkyl; (C₁-C₆)haloalkyl; (C₁-C₆)haloalkoxy, (C₁-C₆)halothioalkoxy, optionally substituted aryl (in particular optionally substituted with one or two (C₁-C₆)alkyl or NO₂); heteroaryl; oxo; NR^{a}R^{b}; COR^{c}; CO₂R^{d}; CONR^{e}R^{f}; OR^{g}; SR^{h}; SO₂Rⁱ; SO₂NR^{j}R^{k}; NR^{l}COR^{m}; RⁿNR^{O}R^{P}; NR^{q}SO₂R^{f}; wherein R^{a} to R^{f} are, independently of one another, H, (C₁-C₆)alkyl, (C₁-C₅)haloalkyl, aryl, (C₁-C₆)alkyl-aryl, heteroaryl or (C₁-C₆)alkyl-heteroaryl; in particular consisting of halogen, (C₁-C₆)alkyl, aryl, oxo, —NH₂, —CO₂H or —OH.

In a specific embodiment, when R² and R³ form together with the nitrogen atom to which they bound an optionally substituted heterocycloalkyl, R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH; in particular, R⁴ is selected in the group consisting of H, (C₁-C₁₂)alkyl and aryl; preferably, R⁴ is H.

In a specific embodiment, when R² and R³ form together with the nitrogen atom to which they bound an optionally substituted heterocycloalkyl, R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH; in particular, R¹ is selected in the group consisting of H, (C₁-C₁₂)alkyl, (C₂-C₁₂) alkenyl, (C₂-C₁₂) alkynyl and aryl; preferably, R¹ is H.

In a specific embodiment, when R² and R³ form together with the nitrogen atom to which they bound an optionally substituted heterocycloalkyl:
R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH;
   and
R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₅)alkyl, aryl, oxo, —NH₂, —CO₂H or —OH.

In a specific embodiment, when R² and R³ form together with the nitrogen atom to which they bound an optionally substituted heterocycloalkyl:
R⁴ is preferably selected in the group consisting of H, (C₁-C₁₂)alkyl and aryl; and
R¹ is preferably selected in the group consisting of H, (C₁-C₁₂)alkyl, (C₂-C₁₂) alkenyl, (C₂-C₁₂) alkynyl and aryl.

In a specific embodiment, when R² and R³ form together with the nitrogen atom to which they bound an optionally substituted heterocycloalkyl:
R⁴ is preferably H; and
R¹ is preferably selected in the group consisting of H, (C₁-C₁₂)alkyl, (C₂-C₁₂) alkenyl, (C₂-C₁₂) alkynyl and aryl; more preferably, R¹ is H.

In a specific embodiment,
X is a methyl, an ethyl or a n-propyl; and/or Y⁰ is —O—; and/or Y¹, Y² and Y³ are independently absent or —O—; and
R² and R³ form together with the nitrogen atom to which they bound an optionally substituted nitrogen-heterocycloalkyl comprising 3 to 10 ring atoms; and
R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₅)alkyl, aryl, oxo, —NH₂, —CO₂H or —OH; and/or R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH.

In a specific embodiment,
X is a methyl, an ethyl or a n-propyl; and/or Y⁰ is —O—; and/or Y¹, Y² and Y³ are independently absent or —O—; and
R² and R³ form together with the nitrogen atom to which they bound an optionally substituted nitrogen-heterocycloalkyl comprising one nitrogen atom and 3 to 10 ring atoms; and
R⁴ is preferably selected in the group consisting of H, (C₁-C₁₂)alkyl and aryl; and/or R¹ is preferably selected in the group consisting of H, (C₁-C₁₂)alkyl, (C₂-C₁₂) alkenyl, (C₂-C₁₂) alkynyl and aryl.

In a specific embodiment,
X is a methyl, an ethyl or a n-propyl; and/or Y⁰ is —O—; and/or Y¹, Y² and Y³ are independently absent or —O—; and
R² and R³ form together with the nitrogen atom to which they bound a nitrogen-heterocycloalkyl comprising one nitrogen atom and 3 to 10 ring atoms; and
R⁴ is preferably H; and/or R¹ is preferably selected in the group consisting of H, (C₁-C₁₂)alkyl, (C₂-C₁₂) alkenyl, (C₂-C₁₂) alkynyl and aryl; more preferably, R¹ is H.

In a specific embodiment, when R² and R³ are independently selected in the group consisting of H, —C(O)OR⁹—, —S(O)₂R¹⁰—, —C(O)R⁹—, —S(O)R¹⁰—, —NR¹¹R¹², optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl (R⁹ to R¹² being as defined above), R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH; in particular, R⁴ is selected in the group consisting of H, (C₁-C₁₂)alkyl and aryl; preferably, R⁴ is H.

In a specific embodiment, when R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl (R⁹ and R¹⁰ being as defined above), R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH; provided that at least one of R¹, R² and R³ is not H, preferably at least one of R² and R³ is not H; in particular, R¹ is selected in the group consisting of H, (C₁-C₁₂)alkyl, (C₂-C₁₂) alkenyl, (C₂-C₁₂) alkynyl and aryl; preferably, R¹ is H.

In a specific embodiment, when R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl; and R⁹ and R¹⁰ are as defined above:
R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH;
   and/or
R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH;
   provided that at least one of R¹, R² and R³ is not H, preferably at least one of R² and R³ is not H.

In a specific embodiment, when R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl; and R⁹ and R¹⁰ are as defined above:
R⁴ is preferably selected in the group consisting of H, (C₁-C₁₂)alkyl and aryl; and/or
R¹ is preferably selected in the group consisting of H, (C₁-C₁₂)alkyl, (C₂-C₁₂) alkenyl, (C₂-C₁₂) alkynyl and aryl;
   provided that at least one of R¹, R² and R³ is not H, preferably at least one of R² and R³ is not H.

In a specific embodiment, when R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl; and R⁹ and R¹⁰ are as defined above:
R⁴ is preferably H; and/or
R¹ is preferably selected in the group consisting of H, (C₁-C₁₂)alkyl, (C₂-C₁₂) alkenyl, (C₂-C₁₂) alkynyl and aryl; more preferably, R¹ is H;
   provided that at least one of R¹, R² and R³ is not H, preferably at least one of R² and R³ is not H.

In a specific embodiment,
X is a methyl, an ethyl or a n-propyl; and/or Y⁰ is —O—; and/or Y¹, Y² and Y³ are independently absent or —O—; and
R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl (R⁹ and R¹⁰ being as defined above); and
R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH; and/or R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH;

provided that at least one of R¹, R² and R³ is not H, preferably at least one of R² and R³ is not H.

In a specific embodiment,
X is a methyl, an ethyl or a n-propyl; and/or Y⁰ is —O—; and/or Y¹, Y² and Y³ are independently absent or —O—; and
R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl (R⁹ and R¹⁰ being as defined above); and
R⁴ is preferably selected in the group consisting of H, (C₁-C₁₂)alkyl and aryl; and/or R¹ is preferably selected in the group consisting of H, (C₁-C₁₂)alkyl, (C₂-C₁₂) alkenyl, (C₂-C₁₂) alkynyl and aryl;

provided that at least one of R¹, R² and R³ is not H, preferably at least one of R² and R³ is not H.

In a specific embodiment,
X is a methyl, an ethyl or a n-propyl; and/or Y⁰ is —O—; and/or Y¹, Y² and Y³ are independently absent or —O—; and
R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl (R⁹ and R¹⁰ being as defined above); and
R⁴ is preferably H; and/or R¹ is preferably selected in the group consisting of H, (C₁-C₁₂)alkyl, (C₂-C₁₂) alkenyl, (C₂-C₁₂) alkynyl and aryl; more preferably, R¹ is H;
   provided that at least one of R¹, R² and R³ is not H, preferably at least one of R² and R³ is not H.

In the context of the invention, the compound of formula (I) for use according to the invention is preferably selected from compounds (001) to (064) and (A1) to (A4) of Table 1 below, or a pharmaceutically acceptable salt and/or solvate thereof. Preferably, the compound of formula (I) for use according to the invention is preferably selected from compounds (A1), (A2), (A3), (A4), (002), (003), (004), (008), (009), (010), (012), (013), (014), (015), (016), (017), (018), (019), (020), (022), (023), (024), (025), (026), (028), (029), (036), (039), (040), (043), (045), (049), (051), (052), (054), (057), (058), (059), (061), (063), and (064) of Table 1. More preferably, the compound of formula (I) for use according to the invention is preferably selected from compounds (001) to (038) of Table 1, or a pharmaceutically acceptable salt and/or solvate thereof. More preferably, the compound of formula (I) for use according to the invention is preferably selected from compounds (003) to (007), (013), (014), (021), (031) and (034) to (038) of Table 1, or a pharmaceutically acceptable salt and/or solvate thereof.

In the context of the invention, the compound of formula (I) for use according to the invention is preferably selected from compounds (001) to (038) and (A1) to (A4) of Table 1 below, or a pharmaceutically acceptable salt and/or solvate thereof. Preferably, the compound of formula (I) for use according to the invention is preferably selected from compounds (001) to (038) of Table 1, or a pharmaceutically acceptable salt and/or solvate thereof. More preferably, the compound of formula (I) for use according to the invention is preferably selected from compounds (003) to (007), (013), (014), (021), (029) and (031) to (038) of Table 1, or a pharmaceutically acceptable salt and/or solvate thereof.

In a specific embodiment, in the compound of formula (I) for use according to the invention, when R⁵, R⁶, R⁷ and R⁸ are H, Y⁰ is —O—, and Y¹, Y² and Y³ are absent, then R¹, R² and R³ are not an optionally substituted (C₂-C₂₄) alkynyl.

In a specific embodiment, the compound of formula (I) for use according to the invention is not a compound (A1), (A2), (A3) or (A4).

### New Compounds

The invention also relates to a compound of general formula (I), or a pharmaceutically acceptable salt, stereoisomer, tautomer or solvate thereof,

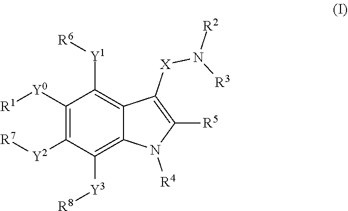

or a pharmaceutically acceptable salt and/or solvate thereof,
wherein
R¹, R², R³ and R⁴ are independently selected in the group consisting of H, —OH, —C(O)OR⁹—, —S(O)₂R¹⁰—, —C(O)R⁹—, —S(O)R¹⁰—, —NR¹¹R¹²—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl, provided that at least one of R² and R³ is not H;
or
R¹ and R⁴ are independently selected in the group consisting of H, OH, —C(O)OR⁹—, —S(O)₂R¹⁰—, —C(O)R⁹—, —S(O)R¹⁰—, —NR¹¹R¹²—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl, and R² and R³ with the nitrogen atom to which they are bound form together a nitrogen-heterocycloalkyl optionally substituted with at least one substituent selected in the group consisting of halogen; CN; NO₂; (C₁-C₆)alkyl; (C₁-C₆)haloalkyl; (C₁-C₆)haloalkoxy, (C₁-C₆)halothioalkoxy, optionally substituted aryl (in particular optionally substituted with one or two (C₁-C₆)alkyl or NO₂); heteroaryl; oxo; NR^{a}R^{b}; COR^{c}; CO₂R^{d}; CONR^{e}R^{f}; OR^{g}; SR^{h}; SO₂Rⁱ; SO₂NR^{j}R^{k}; NR^{l}COR^{m}; RⁿNR^{O}R^{P}; NR^{q}SO₂R^{r}; wherein R^{a} to R^{r} are, independently of one another, H, (C₁-C₆)alkyl, (C₁-C₆)haloalkyl, aryl, (C₁-C₆)alkyl-aryl, heteroaryl or (C₁-C₆)alkyl-heteroaryl; in particular consisting of halogen, CN, NO₂, (C₁-C₆)alkyl, (C₁-C₆)haloalkyl, oxo, NR^{a}R^{b}, COR^{c}, C(O)OR^{d}, CONR^{e}R^{f}, and OR^{g}, wherein R^{a} to R^{g} are, independently of one another, H or (C₁-C₆)alkyl;
X is selected in the group consisting of (C₁-C₁₂)alkyl, —O—(C₁-C₁₂)alkyl-, —C(O)—, —C(O)—(C₁-C₁₂)alkyl-, —NH—C(O)—(C₁-C₁₂)alkyl-, —S—(C₁-C₁₂)alkyl-, —S(O)—(C₁-C₁₂)alkyl-, and —S(O)₂—(C₁-C₁₂)alkyl-; the (C₁-C₁₂)alkyl groups being optionally substituted and/or optionally interrupted by an heteroatom;
Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴—, (C₁-C₂₄)alkyl, —NH—C(O)—NH—, —NH—S(O)—NH—, —NH—S(O₂)—NH—, —S—C(O)—NH—, —O—C(O)—NH—, —O—S(O)—NH—, —O—S(O₂)—NH—, —S—CH₂—C(O)—NH—, —S—CH₂—S(O)—NH—, —S—CH₂—S(O₂)—NH—, —S(O)—CH₂—C(O)—NH—, —S(O₂)—CH₂—C(O)—NH—, —O—CH₂—C(O)—NH—, —O—CH₂—S(O)—NH—, —O—CH₂—S(O₂)—NH—, —NH—CH₂—C(O)—NH—, —NH—CH₂—S(O)—NH—, —NH—CH₂—S(O₂)—NH—, —O—(CH₂)₂—NH—C(O)—, —O—(CH₂)₂—N(CH₃)—C(O)—, —O—(CH₂)₂—NH—S(O)—, —O—(CH₂)₂—N(CH₃)—S(O)—, —O—(CH₂)₂—NH—S(O₂)—, —O—(CH₂)₂—N(CH₃)—S(O₂)—, —S—(CH₂)₂—NH—C(O)—, —S—(CH₂)₂—N(CH₃)—C(O)—, —S—(CH₂)₂—NH—S(O)—, —S—(CH₂)₂—N(CH₃)—S(O)—, —S—(CH₂)₂—NH—S(O₂)—, —S—(CH₂)₂—N(CH₃)—S(O₂)—, —NH—(CH₂)₂—NH—C(O)—, —NH—(CH₂)₂—N(CH₃)—C(O)—, —NH—(CH₂)₂—NH—S(O)—, —NH—(CH₂)₂—N(CH₃)—S(O)—, —NH—(CH₂)₂—NH—S(O₂)—, —NH—(CH₂)₂—N(CH₃)—S(O₂)—, —O—CH₂—CH(CH₂—OH)—NH—C(O)—, —O—CH₂—CH(CH₂—OCH₃)—NH—C(O)—, —O—CH₂—CH(CH₃)—NH—C(O)—, —O—CH(CH₃)—CH₂—NH—C(O)—, —O—CH₂—CH(CH₂—OH)—NH—S(O)—, —O—CH₂—CH(CH₂—OCH₃)—NH—S(O)—, —O—CH₂—CH(CH₃)—NH—S(O)—, —O—CH(CH₃)—CH₂—NH—S(O)—, —O—CH₂—CH(CH₂—OH)—NH—S(O₂)—, —O—CH₂—CH(CH₂—OCH₃)—NH—S(O₂)—, —O—CH₂—CH(CH₃)—NH—S(O₂)—, —O—CH(CH₃)—CH₂—NH—S(O₂)—, —S—CH₂—CH(CH₂—OH)—NH—C(O)—, —S—CH₂—CH(CH₂—OCH₃)—NH—C(O)—, —S—CH₂—CH(CH₃)—NH—C(O)—, —S—CH(CH₃)—CH₂—NH—C(O)—, —S—CH₂—CH(CH₂—OH)—NH—S(O)—, —S—CH₂—CH(CH₂—OCH₃)—NH—S(O)—, —S—CH₂—CH(CH₃)—NH—S(O)—, —S—CH(CH₃)—CH₂—NH—S(O)—, —S—CH₂—CH(CH₂—OH)—NH—S(O₂)—, —S—CH₂—CH(CH₂—OCH₃)—NH—S(O₂)—, —S—CH₂—CH(CH₃)—NH—S(O₂)—, —S—CH(CH₃)—CH₂—NH—S(O₂)—, —NH—CH₂—CH(CH₂—OH)—NH—C(O)—, —NH—CH₂—CH(CH₂—OCH₃)—NH—C(O)—, —NH—CH₂—CH(CH₃)—NH—C(O)—, —NH—CH(CH₃)—CH₂—NH—C(O)—, —NH—CH₂—CH(CH₂—OH)—NH—S(O)—, —NH—CH₂—CH(CH₂—OCH₃)—NH—S(O)—, —NH—CH₂—CH(CH₃)—NH—S(O)—, —NH—CH(CH₃)—CH₂—NH—S(O)—, —NH—CH₂—CH(CH₂—OH)—NH—S(O₂)—, —NH—CH₂—CH(CH₂—OCH₃)—NH—S(O₂)—, —NH—CH₂—CH(CH₃)—NH—S(O₂)—, —NH—CH(CH₃)—CH₂—NH—S(O₂)—, —O—(CH₂)₂—NH—, —S—(CH₂)₂—NH—, and —NH—(CH₂)₂—NH—; the (C₁-C₂₄)alkyl groups being optionally substituted; preferably Y⁰ is not absent, and
R⁵, R⁶, R⁷ and R⁸ are independently selected in the group consisting of H, halogen, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl; and
R⁹ to R¹⁴ are independently selected in the group consisting of optionally substituted (C₁-C₁₂)alkyl, optionally substituted (C₂-C₁₂) alkenyl, optionally substituted (C₂-C₁₂) alkynyl, optionally substituted aryl;
provided that when R⁵, R⁶, R⁷ and R⁸ are H, Y⁰ is —O—, and Y¹, Y² and Y³ are absent, then R¹, R² and R³ are not an optionally substituted (C₂-C₂₄) alkynyl.

In particular, R⁹ is selected in the group consisting of optionally substituted (C₁-C₁₂)alkyl, optionally substituted (C₂-C₁₂) alkenyl, optionally substituted (C₂-C₁₂) alkynyl, and aryl optionally substituted with a (C₁-C₆)alkyl; and
R¹⁰ to R¹⁴ are independently selected in the group consisting of optionally substituted (C₁-C₁₂)alkyl, optionally substituted (C₂-C₁₂) alkenyl, optionally substituted (C₂-C₁₂) alkynyl, optionally substituted aryl.

In the compound of general formula (I), X is selected in the group consisting of (C₁-C₁₂)alkyl, —O—(C₁-C₁₂)alkyl-, —C(O)—, —C(O)—(C₁-C₁₂)alkyl-, —NH—C(O)—(C₁-C₁₂)alkyl-, —S—(C₁-C₁₂)alkyl-, —S(O)—(C₁-C₁₂)alkyl-, and —S(O)₂—(C₁-C₁₂)alkyl-; the (C₁-C₁₂)alkyl groups being optionally substituted and/or optionally interrupted by an heteroatom. Preferably X is selected in the group consisting of (C₁-C₁₂)alkyl, —O—(C₁-C₁₂)alkyl-, —C(O)—, —C(O)—(C₁-C₁₂)alkyl-, —NH—C(O)—(C₁-C₁₂)alkyl-; the (C₁-C₁₂)alkyl groups being optionally substituted and/or optionally interrupted by an heteroatom; the (C₁-C₁₂)alkyl groups being optionally substituted and/or optionally interrupted by an heteroatom. Preferably, X is selected in the group consisting of (C₁-C₆)alkyl, —O—(C₁-C₆)alkyl-, —C(O)—, —C(O)—(C₁-C₆)alkyl- and —NH—C(O)—(C₁-C₆)alkyl-; the (C₁-C₆)alkyl groups being optionally substituted and/or optionally interrupted by an heteroatom. More preferably, X is selected in the group consisting of (C₁-C₃)alkyl, —O—(C₁-C₃)alkyl-, —C(O)—(C₁-C₃)alkyl- and —NH—C(O)—(C₁-C₃)alkyl-; the (C₁-C₃)alkyl groups being optionally substituted and/or optionally interrupted by an heteroatom.

In a specific embodiment, X is a (C₁-C₆)alkyl. Preferably X is a methyl, an ethyl or a n-propyl, more preferably an ethyl.

In the compound of general formula (I), Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴, optionally substituted (C₁-C₂₄)alkyl, —NH—C(O)—NH—, —NH—S(O)—NH—, —NH—S(O₂)—NH—, —S—C(O)—NH—, —O—C(O)—NH—, —O—S(O)—NH—, —O—S(O₂)—NH—, —S—CH₂—C(O)—NH—, —S—CH₂—S(O)—NH—, —S—CH₂—S(O₂)—NH—, —S(O)—CH₂—C(O)—NH—, —S(O₂)—CH₂—C(O)—NH—, —O—CH₂—C(O)—NH—, —O—CH₂—S(O)—NH—, —O—CH₂—S(O₂)—NH—, —NH—CH₂—C(O)—NH—, —NH—CH₂—S(O)—NH—, —NH—CH₂—S(O₂)—NH—, —O—(CH₂)₂—NH—C(O)—, —O—(CH₂)₂—N(CH₃)—C(O)—, —O—(CH₂)₂—NH—S(O)—, —O—(CH₂)₂—N(CH₃)—S(O)—, —O—(CH₂)₂—NH—S(O₂)—, —O—(CH₂)₂—N(CH₃)—S(O₂)—, —S—(CH₂)₂—NH—C(O)—, —S—(CH₂)₂—N(CH₃)—C(O)—, —S—(CH₂)₂—NH—S(O)—, —S—(CH₂)₂—N(CH₃)—S(O)—, —S—(CH₂)₂—NH—S(O₂)—, —S—(CH₂)₂—N(CH₃)—S(O₂)—, —NH—(CH₂)₂—NH—C(O)—, —NH—(CH₂)₂—N(CH₃)—C(O)—, —NH—(CH₂)₂—NH—S(O)—, —NH—(CH₂)₂—N(CH₃)—S(O)—, —NH—(CH₂)₂—NH—S(O₂)—, —NH—(CH₂)₂—N(CH₃)—S(O₂)—, —O—CH₂—CH(CH₂—OH)—NH—C(O)—, —O—CH₂—CH(CH₂—OCH₃)—NH—C(O)—, —O—CH₂—CH(CH₃)—NH—C(O)—, —O—CH(CH₃)—CH₂—NH—C(O)—, —O—CH₂—CH(CH₂—OH)—NH—S(O)—, —O—CH₂—CH(CH₂—OCH₃)—NH—S(O)—, —O—CH₂—CH(CH₃)—NH—S(O)—, —O—CH(CH₃)—CH₂—NH—S(O)—, —O—CH₂—CH(CH₂—OH)—NH—S(O₂)—, —O—CH₂—CH(CH₂—OCH₃)—NH—S(O₂)—, —O—CH₂—CH(CH₃)—NH—S(O₂)—, —O—CH(CH₃)—CH₂—NH—S(O₂)—, —S—CH₂—CH(CH₂—OH)—NH—C(O)—, —S—CH₂—CH(CH₂—OCH₃)—NH—C(O)—, —S—CH₂—CH(CH₃)—NH—C(O)—, —S—CH(CH₃)—CH₂—NH—C(O)—, —S—CH₂—CH(CH₂—OH)—NH—S(O)—, —S—CH₂—CH(CH₂—OCH₃)—NH—S(O)—, —S—CH₂—CH(CH₃)—NH—S(O)—, —S—CH(CH₃)—CH₂—NH—S(O)—, —S—CH₂—CH(CH₂—OH)—NH—S(O₂)—, —S—CH₂—CH(CH₂—OCH₃)—NH—S(O₂)—, —S—CH₂—CH(CH₃)—NH—S(O₂)—, —S—CH(CH₃)—CH₂—NH—S(O₂)—, —NH—CH₂—CH(CH₂—OH)—NH—C(O)—, —NH—CH₂—CH(CH₂—OCH₃)—NH—C(O)—, —NH—CH₂—CH(CH₃)—NH—C(O)—, —NH—CH(CH₃)—CH₂—NH—C(O)—, —NH—CH₂—CH(CH₂—OH)—NH—S(O)—, —NH—CH₂—CH(CH₂—OCH₃)—NH—S(O)—, —NH—CH₂—CH(CH₃)—NH—S(O)—, —NH—CH(CH₃)—CH₂—NH—S(O)—, —NH—CH₂—CH(CH₂—OH)—NH—S(O₂)—, —NH—CH₂—CH(CH₂—OCH₃)—NH—S(O₂)—, —NH—CH₂—CH(CH₃)—NH—S(O₂)—, —NH—CH(CH₃)—CH₂—NH—S(O₂)—, —O—(CH₂)₂—NH—, —S—(CH₂)₂—NH—, and —NH—(CH₂)₂—NH—; the (C₁-C₂₄)alkyl groups being optionally substituted. In particular, Y⁰ is not absent.

In a specific embodiment, in the compound of general formula (I), Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴—, optionally substituted (C₁-C₂₄)alkyl, —NH—C(O)—NH—, —NH—S(O)—NH—, —NH—S(O₂)—NH—, —S—C(O)—NH—, —O—C(O)—NH—, —O—S(O)—NH—, —O—S(O₂)—NH—, —O—(CH₂)₂—NH—, —S—(CH₂)₂—NH—, and —NH—(CH₂)₂—NH—; the (C₁-C₂₄)alkyl groups being optionally substituted. In particular, Y⁰ is not absent.

In a particular embodiment, the compound of general formula (I), Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴—, and optionally substituted (C₁-C₂₄)alkyl; the (C₁-C₂₄)alkyl groups being optionally substituted; and preferably, Y⁰ is not absent. In particular, Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₁₂)alkyl-NH—, —(C₁-C₁₂)alkyl-O—, —(C₁-C₁₂)alkyl-S—, (C₁-C₁₂)alkyl-NR¹³—, —(C₁-C₁₂)alkyl-S(O)—, —(C₁-C₁₂)alkyl-S(O)₂—, —(C₁-C₁₂)alkyl-C(O)—, —(C₁-C₁₂)alkyl-C(O)O—, —(C₁-C₁₂)alkyl-OC(O)—, —(C₁-C₁₂)alkyl-NHC(O)—, —(C₁-C₁₂)alkyl-C(O)NH—, —(C₁-C₁₂)alkyl-C(O)NR¹⁴—, and optionally substituted (C₁-C₁₂)alkyl; the (C₁-C₁₂)alkyl groups being optionally substituted; and preferably, Y⁰ is not absent. More particularly, Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—; —C(O)NR¹⁴— —(C₁-C₆)alkyl-NH—, —(C₁-C₆)alkyl-O—, —(C₁-C₆)alkyl-S—, (C₁-C₆)alkyl-NR¹³—, —(C₁-C₆)alkyl-S(O)—, —(C₁-C₆)alkyl-S(O)₂—, —(C₁-C₆)alkyl-C(O)—, —(C₁-C₆)alkyl-C(O)O—, —(C₁-C₅)alkyl-OC(O)—, —(C₁-C₆)alkyl-NHC(O)—, —(C₁-C₆)alkyl-C(O)NH—, —(C₁-C₆)alkyl-C(O)NR¹⁴—, and optionally substituted (C₁-C₆)alkyl; the (C₁-C₆)alkyl groups being optionally substituted; and preferably, Y⁰ is not absent. Preferably, Y⁰, Y¹, Y² and Y³ are independently absent or selected from —O—, and optionally substituted (C₁-C₆)alkyl; and preferably, Y⁰ is not absent. In particular, Y⁰ is not absent.

In a specific embodiment, Y⁰, Y¹, Y² and Y³ are independently absent or —O—. Preferably Y⁰ is —O— and, Y¹, Y² and Y³ are independently absent or —O—, preferably absent.

In a specific embodiment, in the compound of general formula (I), X is a (C₁-C₆)alkyl, Y⁰ is —O—, and Y¹, Y² and Y³ are independently absent or —O—.

In a particular embodiment, X is a methyl, an ethyl or a n-propyl, Y⁰ is —O—, and Y¹, Y² and Y³ are independently absent or —O—. Preferably, X is a methyl, an ethyl or a n-propyl, Y⁰ is —O—, and Y¹, Y² and Y³ are absent. More preferably, X is an ethyl, Y⁰ is —O—, and Y¹, Y² and Y³ are absent.

In the compound of general formula (I), R⁵, R⁶, R⁷ and R⁸ independently selected in the group consisting of H, halogen, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl. Preferably, at least one of R⁵, R⁶, R⁷ and R⁸ is not H.

In a specific embodiment, R⁵, R⁶, R⁷ and R⁸ are independently selected in the group consisting of H, halogen, optionally substituted (C₁-C₂₄)alkyl, optionally substituted (C₂-C₂₄) alkenyl, optionally substituted (C₂-C₂₄) alkynyl. In particular, R⁵, R⁶, R⁷ and R⁸ are independently selected in the group consisting of H, halogen, optionally substituted (C₁-C₁₂)alkyl, optionally substituted (C₂-C₁₂) alkenyl, optionally substituted (C₂-C₁₂) alkynyl. More particularly, R⁵, R⁶, R⁷ and R⁸ are independently selected in the group consisting of H, halogen, optionally substituted (C₁-C₅)alkyl, optionally substituted (C₂-C₆) alkenyl, optionally substituted (C₂-C₆) alkynyl. Preferably R⁵, R⁶, R⁷ and R⁸ are independently selected in the group consisting of H, halogen, haloalkyl, or (C₁-C₂₄)alkyl. More preferably R⁵, R⁶, R⁷ and R⁸ are independently selected in the group consisting of H, halogen, haloalkyl such as CF₃, or (C₁-C₅)alkyl. Preferably, at least one of R⁵, R⁶, R⁷ and R⁸ is not H.

In a specific embodiment, three of R⁵, R⁶, R⁷ and R⁸ are H, and the last one is as defined above except H.

In another specific embodiment, two of R⁵, R⁶, R⁷ and R⁸ are H, and two are as defined above except H.

In another specific embodiment, three of R⁵, R⁶, R⁷ and R⁸ are H, and one is as defined above except H.

In another specific embodiment, R⁵, R⁶, R⁷ and R⁸ are H.

In another specific embodiment, R⁵, R⁶, R⁷ and R⁸ are as defined above but not H.

In a particular embodiment, one or two of R⁵, R⁶, R⁷ and R⁸ are selected in the group consisting of H, halogen, haloalkyl such as CF₃, or (C₁-C₆)alkyl, and the others are H.

In a particular embodiment, one or two of R⁵, R⁶, R⁷ and R⁸ are selected in the group consisting of halogen, haloalkyl such as CF₃, or (C₁-C₅)alkyl, and the others are H.

In a specific embodiment, Y⁰ is —O—, and Y¹, Y² and Y³ are absent, and:
three of R⁵, R⁶, R⁷ and R⁸ are H, and the last one is as defined above except H; or
two of R⁵, R⁶, R⁷ and R⁸ are H, and two are as defined above except H; or
three of R⁵, R⁶, R⁷ and R⁸ are H, and one is as defined above except H; or
R⁵, R⁶, R⁷ and R⁸ are H; or
R⁵, R⁶, R⁷ and R⁸ are as defined above but not H.

In a particular embodiment, Y⁰ is —O—, and Y¹, Y² and Y³ are absent, one or two of R⁵, R⁶, R⁷ and R⁸ are selected in the group consisting of H, halogen, haloalkyl such as CF₃, or (C₁-C₆)alkyl, and the others are H.

In a particular embodiment, Y⁰ is —O—, and Y¹, Y² and Y³ are absent, one or two of R⁵, R⁶, R⁷ and R⁸ are selected in the group consisting of halogen, haloalkyl such as CF₃, or (C₁-C₆)alkyl, and the others are H.

In the above embodiment, when R⁵, R⁶, R⁷ and R⁸ are H, and Y¹, Y² and Y³ are absent, R¹, R² and R³ are not an optionally substituted (C₂-C₂₄) alkynyl. The new compound of general formula (I) cannot be a compound (A1), (A2), (A3) or (A4).

In one embodiment of the compound of general formula (I), R¹, R², R³ and R⁴ are independently selected in the group consisting of H, OH, —C(O)OR⁹—, —S(O)₂R¹⁰—, —C(O)R⁹—, —S(O)R¹⁰—, —NR¹¹R¹²—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl, optionally substituted (C₁-C₆)alkyl-aryl, and optionally substituted heteroaryl, provided that at least one of R² and R³ is not H.

In one particular embodiment of the compound of general formula (I), R¹, R², R³ and R⁴ are independently selected in the group consisting of H, OH, —C(O)OR⁹—, —S(O)₂R¹⁰—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl, optionally substituted (C₁-C₆)alkyl-aryl, and optionally substituted heteroaryl, provided that at least one of R² and R³ is not H.

In this definition, “optionally substituted” preferably means non-substituted or substituted with at least one, for example 1, 2 or 3, substituent(s) which may be selected in the group consisting of halogen; CN; NO₂; (C₁-C₆)alkyl; (C₁-C₆)haloalkyl; (C₁-C₅)haloalkoxy, (C₁-C₅)halothioalkoxy, optionally substituted aryl (in particular optionally substituted with one or two (C₁-C₆)alkyl or NO₂); heteroaryl; halogeno-heteroaryl, in particular halogeno-furanyl; oxo; NR^{a}R^{b}; COR^{c}; CO₂R^{d}; CONR^{e}R^{f}; OR^{g}; SR^{h}; SO₂Rⁱ; SO₂NR^{j}R^{k}; NR^{l}COR^{m}; RⁿNR^{O}R^{P}; NR^{q}SO₂R^{r}; wherein R^{a} to R^{r} are, independently of one another, H, (C₁-C₅)alkyl, (C₁-C₅)haloalkyl, aryl, (C₁-C₆)alkyl-aryl, heteroaryl or (C₁-C₅)alkyl-heteroaryl; in particular consisting of halogen; CN; NO₂; (C₁-C₆)alkyl; (C₁-C₆)haloalkyl; aryl optionally substituted with one or two (C₁-C₆)alkyl or NO₂; heteroaryl; oxo; NR^{a}R^{b}; COR^{c}; CO₂R^{d}; CONR^{e}R^{f}; and OR^{g}; wherein R^{a} to R^{g} are, independently of one another, H, (C₁-C₆)alkyl, (C₁-C₆)haloalkyl, aryl, (C₁-C₆)alkyl-aryl, heteroaryl or (C₁-C₆)alkyl-heteroaryl.

In a specific embodiment, R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₅)alkyl, aryl, oxo, —NH₂, —CO₂H or —OH. In particular, R¹ is selected in the group consisting of H, (C₁-C₁₂)alkyl, (C₂-C₁₂) alkenyl, (C₂-C₁₂) alkynyl and aryl. Preferably, R¹ is H.

In a specific embodiment, R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH. In particular, R⁴ is selected in the group consisting of H, (C₁-C₁₂)alkyl and aryl. Preferably, R⁴ is H.

In a specific embodiment, R² and R³ are independently selected in the group consisting of H, OH, —C(O)OR⁹—, —S(O)₂R¹⁰—, —C(O)R⁹—, —S(O)R¹⁰—, —NR¹¹R¹²—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl, provided that at least one of R² and R³ is not H; or R² and R³ form together with the nitrogen atom to which they bound an optionally substituted heterocycloalkyl. In this definition, “optionally substituted” preferably means non-substituted or substituted with at least one, for example 1, 2 or 3, substituent(s) which may be preferably halogen; CN; NO₂; (C₁-C₆)alkyl; (C₁-C₆)haloalkyl; (C₁-C₆)haloalkoxy, (C₁-C₆)halothioalkoxy, optionally substituted aryl (in particular optionally substituted with one or two (C₁-C₆)alkyl or NO₂); heteroaryl; halogeno-heteroaryl, in particular halogeno-furanyl; oxo; NR^{a}R^{b}; COR^{c}; CO₂R^{d}; CONR^{e}R^{f}; OR^{g}; SR^{h}; SO₂Rⁱ; SO₂NR^{j}R^{k}; NR^{l}COR^{m}; RⁿNR^{O}R^{P}; NR^{q}SO₂R^{r}; wherein R^{a} to R^{r} are, independently of one another, H, (C₁-C₆)alkyl, (C₁-C₆)haloalkyl, aryl, (C₁-C₆)alkyl-aryl, heteroaryl or (C₁-C₆)alkyl-heteroaryl; in particular (C₁-C₆)alkyl; halogen; CN; NO₂; (C₁-C₅)haloalkyl; phenyl optionally substituted with one or two (C₁-C₆)alkyl or NO₂; heteroaryl optionally substituted with —OH; oxo; NR^{a}R^{b}; (C₁-C₁₂)alkyl-NR^{a}R^{b}; COR^{c}; CO₂R^{d}; CONR^{e}R^{f}; and OR^{g}; wherein R^{a} to R^{g} are, independently of one another, H, (C₁-C₅)alkyl, (C₁-C₅)haloalkyl, aryl, (C₁-C₆)alkyl-aryl, heteroaryl or (C₁-C₆)alkyl-heteroaryl.

In the compound of general formula (I), when one or more of R¹, R², R³ and R⁴ is —C(O)OR⁹—, —S(O)₂R¹⁰—, —C(O)R⁹—, —S(O)R¹⁰—, or —NR¹¹R¹²—, preferably —C(O)OR⁹— or —S(O)₂R¹⁰—, R⁹ to R¹² are independently selected in the group consisting of optionally substituted (C₁-C₁₂)alkyl, optionally substituted (C₂-C₁₂) alkenyl, optionally substituted (C₂-C₁₂) alkynyl, and optionally substituted aryl.

In the compound of general formula (I), when one or more of R¹, R², R³ and R⁴ is —C(O)OR⁹—, —S(O)₂R¹⁰—, —C(O)R⁹—, —S(O)R¹⁰—, or —NR¹¹R¹²—, preferably —C(O)OR⁹— or —S(O)₂R¹⁰—, R⁹ is selected in the group consisting of optionally substituted (C₁-C₁₂)alkyl, optionally substituted (C₂-C₁₂) alkenyl, optionally substituted (C₂-C₁₂) alkynyl, and aryl optionally substituted with a (C₁-C₆)alkyl; and R¹⁰ to R¹² are independently selected in the group consisting of optionally substituted (C₁-C₁₂)alkyl, optionally substituted (C₂-C₁₂) alkenyl, optionally substituted (C₂-C₁₂) alkynyl, and optionally substituted aryl.

In this definition, “optionally substituted” preferably means non-substituted or substituted with at least one, for example 1, 2 or 3, substituent(s) which may be preferably (C₁-C₆)alkyl.

In a specific embodiment, R⁹ to R¹² are independently selected in the group consisting of optionally substituted (C₁-C₆)alkyl, optionally substituted (C₂-C₆) alkenyl, optionally substituted (C₂-C₆) alkynyl, and optionally substituted aryl.

In particular, R⁹ is selected in the group consisting of optionally substituted (C₁-C₆)alkyl, optionally substituted (C₂-C₆) alkenyl, optionally substituted (C₂-C₆) alkynyl, and aryl optionally substituted with a (C₁-C₆)alkyl such as methyl; and R¹⁰ to R¹² are independently selected in the group consisting of optionally substituted (C₁-C₆)alkyl, optionally substituted (C₂-C₆) alkenyl, optionally substituted (C₂-C₆) alkynyl, and optionally substituted aryl.

Preferably, R⁹ to R¹² are independently selected in the group consisting of (C₁-C₆)alkyl, (C₂-C₆) alkenyl, (C₂-C₆) alkynyl, aryl or aryl substituted with a (C₁-C₆)alkyl such as methyl.

In another embodiment of the compound of general formula (I), R¹ and R⁴ are independently selected in the group consisting of H, OH, —C(O)OR⁹—, —S(O)₂R¹⁰—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl; and R² and R³ form together with the nitrogen atom to which they bound an optionally substituted heterocycloalkyl.

In a specific embodiment, the optionally substituted heterocycloalkyl formed by R² and R³ with the nitrogen atom to which they are bound form together is preferably an optionally substituted nitrogen-heterocycloalkyl comprising 3 to 10 ring atoms; preferably an optionally substituted nitrogen-heterocycloalkyl comprising one nitrogen atom and 3 to 10 ring atoms. In particular, the nitrogen-heterocycloalkyl is non substituted or substituted with one or more substituents selected in the list consisting of a halogen; CN; NO₂; (C₁-C₆)alkyl; (C₁-C₆)haloalkyl; (C₁-C₆)haloalkoxy, (C₁-C₆)halothioalkoxy, optionally substituted aryl (in particular optionally substituted with one or two (C₁-C₆)alkyl or NO₂); heteroaryl; oxo; NR^{a}R^{b}; COR^{c}; CO₂R^{d}; CONR^{e}R^{f}; OR^{g}; SR^{h}; SO₂Rⁱ; SO₂NR^{j}R^{k}; NR^{l}COR^{m}; RⁿNR^{O}R^{P}; NR^{q}SO₂R^{r}; wherein R^{a} to R^{r} are, independently of one another, H, (C₁-C₆)alkyl, (C₁-C₆)haloalkyl, aryl, (C₁-C₆)alkyl-aryl, heteroaryl or (C₁-C₆)alkyl-heteroaryl; in particular consisting of halogens, (C₁-C₆)alkyl, aryl, oxo, —NH₂, —CO₂H or —OH.

In a specific embodiment, when R² and R³ form together with the nitrogen atom to which they bound an optionally substituted heterocycloalkyl, R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH; in particular, R⁴ is selected in the group consisting of H, (C₁-C₁₂)alkyl and aryl; preferably, R⁴ is H.

In a specific embodiment, when R² and R³ form together with the nitrogen atom to which they bound an optionally substituted heterocycloalkyl, R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH; in particular, R¹ is selected in the group consisting of H, (C₁-C₁₂)alkyl, (C₂-C₁₂) alkenyl, (C₂-C₁₂) alkynyl and aryl; preferably, R¹ is H.

In a specific embodiment, when R² and R³ form together with the nitrogen atom to which they bound an optionally substituted heterocycloalkyl:
R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH;
   and
R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₅)alkyl, aryl, oxo, —NH₂, —CO₂H or —OH.

In a specific embodiment, when R² and R³ form together with the nitrogen atom to which they bound an optionally substituted heterocycloalkyl:
R⁴ is preferably selected in the group consisting of H, (C₁-C₁₂)alkyl and aryl; and
R¹ is preferably selected in the group consisting of H, (C₁-C₁₂)alkyl, (C₂-C₁₂) alkenyl, (C₂-C₁₂) alkynyl and aryl.

In a specific embodiment, when R² and R³ form together with the nitrogen atom to which they bound an optionally substituted heterocycloalkyl:
R⁴ is preferably H; and
R¹ is preferably selected in the group consisting of H, (C₁-C₁₂)alkyl, (C₂-C₁₂) alkenyl, (C₂-C₁₂) alkynyl and aryl; more preferably, R¹ is H.

In a specific embodiment,
X is a methyl, an ethyl or a n-propyl; and/or Y⁰ is —O—; and/or Y¹, Y² and Y³ are independently absent or —O—; and
R² and R³ form together with the nitrogen atom to which they bound an optionally substituted nitrogen-heterocycloalkyl comprising 3 to 10 ring atoms; and
R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH; and/or R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₅)alkyl, aryl, oxo, —NH₂, —CO₂H or —OH.

In a specific embodiment,
X is a methyl, an ethyl or a n-propyl; and/or Y⁰ is —O—; and/or Y¹, Y² and Y³ are independently absent or —O—; and
R² and R³ form together with the nitrogen atom to which they bound an optionally substituted nitrogen-heterocycloalkyl comprising one nitrogen atom and 3 to 10 ring atoms; and
R⁴ is preferably selected in the group consisting of H, (C₁-C₁₂)alkyl and aryl; and/or R¹ is preferably selected in the group consisting of H, (C₁-C₁₂)alkyl, (C₂-C₁₂) alkenyl, (C₂-C₁₂) alkynyl and aryl.

In a specific embodiment,
X is a methyl, an ethyl or a n-propyl; and/or Y⁰ is —O—; and/or Y¹, Y² and Y³ are independently absent or —O—; and
R² and R³ form together with the nitrogen atom to which they bound a nitrogen-heterocycloalkyl comprising one nitrogen atom and 3 to 10 ring atoms; and
R⁴ is preferably H; and/or R¹ is preferably selected in the group consisting of H, (C₁-C₁₂)alkyl, (C₂-C₁₂) alkenyl, (C₂-C₁₂) alkynyl and aryl; more preferably, R¹ is H.

In a specific embodiment, when R² and R³ are independently selected in the group consisting of H, —C(O)OR⁹—, —S(O)₂R¹⁰—, —C(O)R⁹—, —S(O)R¹⁰—, —NR¹¹R¹²—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl (R⁹ to R¹² being as defined above), R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH; in particular, R⁴ is selected in the group consisting of H, (C₁-C₁₂)alkyl and aryl; preferably, R⁴ is H.

In a specific embodiment, when R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl (R⁹ and R¹⁰ being as defined above), R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH; provided that at least one of R² and R³ is not H; in particular, R¹ is selected in the group consisting of H, (C₁-C₁₂)alkyl, (C₂-C₁₂) alkenyl, (C₂-C₁₂) alkynyl and aryl; preferably, R¹ is H.

In a specific embodiment, when R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl; and R⁹ and R¹⁰ are as defined above:
R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₅)alkyl, aryl, oxo, —NH₂, —CO₂H or —OH;
   and/or
R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH;
   provided that at least one of R² and R³ is not H.

In a specific embodiment, when R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl; and R⁹ and R¹⁰ are as defined above:
R⁴ is preferably selected in the group consisting of H, (C₁-C₁₂)alkyl and aryl; and/or
R¹ is preferably selected in the group consisting of H, (C₁-C₁₂)alkyl, (C₂-C₁₂) alkenyl, (C₂-C₁₂) alkynyl and aryl;
provided that at least one of R² and R³ is not H.

In a specific embodiment, when R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl; and R⁹ and R¹⁰ are as defined above:
R⁴ is preferably H; and/or
R¹ is preferably selected in the group consisting of H, (C₁-C₁₂)alkyl, (C₂-C₁₂) alkenyl, (C₂-C₁₂) alkynyl and aryl; more preferably, R¹ is H;
provided that at least one of R² and R³ is not H.

In a specific embodiment,
X is a methyl, an ethyl or a n-propyl; and/or Y⁰ is —O—; and/or Y¹, Y² and Y³ are independently absent or —O—; and
R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl (R⁹ and R¹⁰ being as defined above); and
R⁴ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH; and/or R¹ is preferably selected in the group consisting of H, (C₁-C₂₄)alkyl, (C₂-C₂₄) alkenyl, (C₂-C₂₄) alkynyl, (C₃-C₁₂) cycloalkyl and aryl; said alkyl, alkenyl, alkynyl, aryl or cycloalkyl group being optionally substituted with one or more halogens, (C₁-C₆)alkyl, aryl, oxo, —NH₂, —CO₂H or —OH;

provided that at least one of R² and R³ is not H.

In a specific embodiment,
X is a methyl, an ethyl or a n-propyl; and/or Y⁰ is —O—; and/or Y¹, Y² and Y³ are independently absent or —O—; and
R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl (R⁹ and R¹⁰ being as defined above); and
R⁴ is preferably selected in the group consisting of H, (C₁-C₁₂)alkyl and aryl; and/or R¹ is preferably selected in the group consisting of H, (C₁-C₁₂)alkyl, (C₂-C₁₂) alkenyl, (C₂-C₁₂) alkynyl and aryl;

provided that at least one of R² and R³ is not H.

In a specific embodiment,
X is a methyl, an ethyl or a n-propyl; and/or Y⁰ is —O—; and/or Y¹, Y² and Y³ are independently absent or —O—; and
R² and R³ are independently selected in the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl (R⁹ and R¹⁰ being as defined above); and
R⁴ is preferably H; and/or R¹ is preferably selected in the group consisting of H, (C₁-C₁₂)alkyl, (C₂-C₁₂) alkenyl, (C₂-C₁₂) alkynyl and aryl; more preferably, R¹ is H;
   provided that at least one of R² and R³ is not H.

In the context of the invention, the new compound of formula (I) according to the invention is preferably selected from compounds (001) to (064) of Table 1 below, or a pharmaceutically acceptable salt and/or solvate thereof. Preferably, the new compound of formula (I) for use according to the invention is selected from compounds (002), (003), (004), (008), (009), (010), (012), (013), (014), (015), (016), (017), (018), (019), (020), (022), (023), (024), (025), (026), (028), (029), (036), (039), (040), (043), (045), (049), (051), (052), (054), (057), (058), (059), (061), (063), and (064) of Table 1. Preferably, the new compound of formula (I) for use according to the invention is preferably selected from compounds (003) to (007), (013), (014), (021), (029), (031) and (034) to (038) of Table 1, or a pharmaceutically acceptable salt and/or solvate thereof.

In the context of the invention, the new compound of formula (I) according to the invention is preferably selected from compounds (001) to (038) of Table 1 below, or a pharmaceutically acceptable salt and/or solvate thereof. Preferably, the new compound of formula (I) for use according to the invention is preferably selected from compounds (003) to (007), (013), (014), (021), (029), (031) to (038) of Table 1, or a pharmaceutically acceptable salt and/or solvate thereof.

The invention also relates to a pharmaceutical composition comprising a compound of general formula (I) as defined above, especially one selected from compounds (001) to (064) as disclosed in Table 1, or a pharmaceutically acceptable salt and/or solvate thereof.

The invention also relates to a pharmaceutical composition comprising a compound of general formula (I) as defined above, especially one selected from compounds (001) to (038) as disclosed in Table 1, or a pharmaceutically acceptable salt and/or solvate thereof.

The invention also relates to a compound of general formula (I) as defined above, especially one selected from compounds (001) to (064) as disclosed in Table 1, or a pharmaceutically acceptable salt and/or solvate thereof, or a pharmaceutical composition comprising it, for use as a drug.

The invention also relates to a compound of general formula (I) as defined above, especially one selected from compounds (001) to (038) as disclosed in Table 1, or a pharmaceutically acceptable salt and/or solvate thereof, or a pharmaceutical composition comprising it, for use as a drug.

The invention also relates to the use of a compound of general formula (I) as defined above, especially one selected from compounds (001) to (064) as disclosed in Table 1, or a pharmaceutically acceptable salt and/or solvate thereof, or a pharmaceutical composition comprising it, for the manufacture of a medicament.

The invention also relates to the use of a compound of general formula (I) as defined above, especially one selected from compounds (001) to (038) as disclosed in Table 1, or a pharmaceutically acceptable salt and/or solvate thereof, or a pharmaceutical composition comprising it, for the manufacture of a medicament.

The invention further relates to a method for treating a disease or disorder in a subject in need thereof, comprising administering a therapeutically effective amount of a compound of general formula (I) as defined above, especially one selected from compounds (001) to (064) as disclosed in Table 1, or a pharmaceutically acceptable salt and/or solvate thereof, or a pharmaceutical composition comprising it, to said subject.

The invention further relates to a method for treating a disease or disorder in a subject in need thereof, comprising administering a therapeutically effective amount of a compound of general formula (I) as defined above, especially one selected from compounds (001) to (038) as disclosed in Table 1, or a pharmaceutically acceptable salt and/or solvate thereof, or a pharmaceutical composition comprising it, to said subject.

The present invention also relates to a kit of parts and/or a combination comprising (i) a compound of general formula (I) according to the invention, especially one selected from compounds (001) to (064) as disclosed in Table 1, or a pharmaceutical acceptable salt thereof, and (ii) an additional active ingredient, for separate, sequential or simultaneous use.

The present invention also relates to a kit of parts and/or a combination comprising (i) a compound of general formula (I) according to the invention, especially one selected from compounds (001) to (038) as disclosed in Table 1, or a pharmaceutical acceptable salt thereof, and (ii) an additional active ingredient, for separate, sequential or simultaneous use.

The invention also relates to a compound of general formula (I) as defined above, especially one of those as disclosed in Table 1, or a pharmaceutically acceptable salt and/or solvate thereof, or a pharmaceutical composition comprising it, for use in preventing or treating iron- and/or copper-associated disorders, in particular iron and/or copper overload-associated disorders; or metal-dependent cell death associated disorders.

The invention also relates to the use of a compound of general formula (I) as defined above, especially one of those as disclosed in Table 1, or a pharmaceutically acceptable salt and/or solvate thereof, or a pharmaceutical composition comprising it, for the manufacture of a medicament for preventing or treating iron- and/or copper-associated disorders, in particular iron and/or copper overload-associated disorders; or metal-dependent cell death associated disorders.

The invention further relates to a method for preventing or treating iron- and/or copper-associated disorders, in particular iron and/or copper overload-associated disorders; or metal-dependent cell death associated disorders in a subject in need thereof, comprising administering a therapeutically effective amount of a compound of general formula (I) as defined above, especially one of those as disclosed in Table 1, or a pharmaceutically acceptable salt and/or solvate thereof, or a pharmaceutical composition comprising it, to said subject.

In a specific embodiment, the metalloptosis associated disorders are disorders associated to ferroptosis and/or cuproptosis. Advantageously, the metalloptosis associated disorders are selected among Hemorrhagic stroke; Ischemic Heart Injury; Ischemic Renal Injury; Ischaemia-reperfusion injury (IRI) such as IRI occurring during organ transplantation ((liver, lung, heart, kidneys, pancreas, intestine); Neurodegenerative diseases selected from Huntington Disease, Dementia, and amyotrophic lateral sclerosis (ALS); aging related diseases or disorders; sepsis; ishemia-reperfusion; and Diabetes. In particular, the use of the compound according to the invention is specifically disclosed below in the specification.

In a specific embodiment, the iron- and/or copper-associated disorders are selected among HFE-related hematochromatosis; HFE-related non hematochromatosis; congenital atransferrinenemia; iron-loading associated anemias such as thalassemia, myelodysplasia and hematopoietic stem-cell transplantation-associated disorders; chronic liver diseases; chronic inflammation linked to cancer; autoimmune or inflammatory diseases; neurodegeneration with brain iron accumulation-associated diseases such as Wilson's Disease; and polygenic neurodegenerative-associated diseases such as Parkinson's Disease and Alzheimer Disease.

### Therapeutic Uses

As illustrated by examples, the inventors have demonstrated the therapeutic interest of the new compounds of the invention. Accordingly, the present invention relates to a pharmaceutical or veterinary composition comprising any new compound according to the present invention, or a pharmaceutically acceptable salt and/or solvate thereof. Preferably, the pharmaceutical or veterinary composition further comprises a pharmaceutically or veterinary acceptable carrier or excipient. The present invention relates to the use of any new compound according to the invention, or a pharmaceutically acceptable salt and/or solvate thereof, as a drug or as a medicine. The invention further relates to a method for treating a disease in a subject, wherein a therapeutically effective amount of any new compound according to the invention, or a pharmaceutically acceptable salt and/or solvate thereof, is administered to said subject in need thereof. The invention also relates to the use of any new compound according to the invention, or a pharmaceutically acceptable salt and/or solvate thereof, for the manufacture of a medicine. The invention also relates to a pharmaceutical composition comprising any new compound according to the invention or a pharmaceutically acceptable salt and/or solvate thereof, for use as a drug. The terms “any new compound according to the invention” or ““any new compound of general formula (I)” mean herein any compounds as defined in the section “New compounds” herein above. The terms “any compound according to the invention” or “any compound of general formula (I)” mean herein any compounds of general formula (I) as defined in the present specification, in particular in the sections “Compounds for use” and “New compounds” herein above.

The invention relates to any compounds of general formula (I) according to the invention, especially one of those as disclosed in Table 1, or a pharmaceutically acceptable salt and/or solvate thereof, or a pharmaceutical composition comprising it for use preventing and/or treating metalloptosis associated disorders. It further relates to the use of any compounds of general formula (I) according to the invention, especially one of those as disclosed in Table 1, or a pharmaceutically acceptable salt and/or solvate thereof, or a pharmaceutical composition comprising it for the manufacture of a medicine for use preventing and/or treating metalloptosis associated disorders. It also relates to a method for preventing and/or treating metalloptosis associated disorders in a subject in need thereof, comprising administering a therapeutically effective amount of any compounds of general formula (I) according to the invention, especially one of those as disclosed in Table 1, or a pharmaceutically acceptable salt and/or solvate thereof, or a pharmaceutical composition comprising it to said subject.

In the context if the invention, metalloptosis associated disorders are metal-dependent cell death associated disorders. In particular, metalloptosis associated disorders are disorders associated to ferroptosis and/or cuproptosis.

In the context of the invention, the metalloptosis associated disorders are selected among Hemorrhagic stroke; Ischemic Heart Injury; Ischemic Renal Injury; Ischaemia-reperfusion injury (IRI) such as IRI occurring during organ transplantation ((liver, lung, heart, kidneys, pancreas, intestine); Neurodegenerative diseases selected from Huntington Disease, Dementia, and amyotrophic lateral sclerosis (ALS); aging related diseases or disorders; sepsis; ishemia-reperfusion; and Diabetes.

In another embodiment, the invention relates to any new compounds of general formula (I) according to the invention, or a pharmaceutically acceptable salt and/or solvate thereof, or a pharmaceutical composition comprising it for use for preventing and/or treating iron- and/or copper-associated disorders. It further relates to the use of any new compounds of general formula (I) according to the invention, or a pharmaceutically acceptable salt and/or solvate thereof, or a pharmaceutical composition comprising it for the manufacture of a medicine for use for preventing and/or treating iron- and/or copper-associated disorders. It also relates to a method for preventing and/or treating iron- and/or copper-associated disorders in a subject in need thereof, comprising administering a therapeutically effective amount of any new compounds of general formula (I) according to the invention, or a pharmaceutically acceptable salt and/or solvate thereof, or a pharmaceutical composition comprising it to said subject.

In the context of the invention, the iron- and/or copper-associated disorders are selected among HFE-related hematochromatosis; non HFE-related hematochromatosis; congenital atransferrinenemia; iron-loading associated anemias such as thalassemia, myelodysplasia and hematopoietic stem-cell transplantation-associated disorders; chronic liver diseases; chronic inflammation linked to cancer; autoimmune or inflammatory diseases; neurodegeneration with brain copper- or iron accumulation-associated diseases such as Wilson's Disease; and polygenic neurodegenerative-associated diseases such as Parkinson's Disease and Alzheimer Disease.

In particular, the iron-associated disorders are selected among HFE-related hematochromatosis; non HFE-related hematochromatosis; congenital atransferrinenemia; iron-loading associated anemias such as thalassemia, myelodysplasia and hematopoietic stem-cell transplantation-associated disorders; chronic liver diseases; chronic inflammation linked to cancer; autoimmune or inflammatory diseases; neurodegeneration with brain iron accumulation-associated diseases such as Wilson's Disease; and polygenic neurodegenerative-associated diseases such as Parkinson's Disease and Alzheimer Disease.

In particular, the copper-associated disorders are selected among HFE-related hematochromatosis; non HFE-related hematochromatosis; congenital atransferrinenemia; iron-loading associated anemias such as thalassemia, myelodysplasia and hematopoietic stem-cell transplantation-associated disorders; chronic liver diseases; chronic inflammation linked to cancer; autoimmune or inflammatory diseases; neurodegeneration with brain copper accumulation-associated diseases such as Wilson's Disease; and polygenic neurodegenerative-associated diseases such as Parkinson's Disease and Alzheimer Disease.

HFE-related hematochromatosis may notably be due to C282Y homozygosity or C282/H63D heterozygosity. Non HFE-related hematochromatosis include for example juvenile hemochromatosis type 2A or 2B, or may be due to Mutated transferrin receptor 2 or Mutated ferroportin 1 gene.

Neurodegeneration with brain iron accumulation-associated diseases include aceruloplasminemia, neuroferritinopathy, pantothenate kinase-associated neurodegenration, Wilson's disease and Beta-propeller Protein-Associated Neurodegeneration (BPAN).

Polygenic neurodegenerative disorders include Parkinson's disease and Alzheimer's disease. In particular, the iron overload-associated disorder is iron-loading related anemia, such as thalassemia, myelodysplasy, aplastic anemia, Blackfan diamond anemia, congenital dyserythopoietic anemia, chronic hemoytic anemia, in particular sickel cell disease, hematopoietic stem-cell transplantation-related disorder and chronic liver disease including viral hepatitis, alcoholic hepatitis, steatohepatitis (NASH), dysmetabolic iron overload syndrome.

### Pharmaceutical Compositions

The pharmaceutical compositions contemplated herein may include a pharmaceutically acceptable carrier in addition to the active ingredient(s). The term “pharmaceutically acceptable carrier” is meant to encompass any carrier (e.g., support, substance, solvent, etc.) which does not interfere with effectiveness of the biological activity of the active ingredient(s) and that is not toxic to the host to which it is administered. For example, for parental administration, the active compounds(s) may be formulated in a unit dosage form for injection in vehicles such as saline, dextrose solution, serum albumin and Ringer's solution.

The pharmaceutical composition can be formulated as solutions in pharmaceutically compatible solvents or as emulsions, suspensions or dispersions in suitable pharmaceutical solvents or vehicle, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations suitable for parental administration conveniently comprise a sterile oily or aqueous preparation of the active ingredient which is preferably isotonic with the blood of the recipient. Every such formulation can also contain other pharmaceutically compatible and nontoxic auxiliary agents, such as, e.g. stabilizers, antioxidants, binders, dyes, emulsifiers or flavoring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions are advantageously applied by injection or intravenous infusion of suitable sterile solutions or as oral dosage by the digestive tract. Methods for the safe and effective administration of most of these chemotherapeutic agents are known to those skilled in the art. In addition, their administration is described in the standard literature. The pharmaceutical or veterinary composition as disclosed herein may further comprise an additional active ingredient or drug.

Further aspects and advantages of the present invention will be described in the following examples, which should be regarded as illustrative and not limiting.

### EXAMPLES

TABLE 1

| | |
|---|---|
| 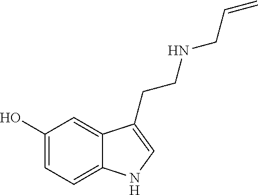 [SMILES: C=CCNCCc1c[nH]c2ccc(O)cc12] | (001) |
| 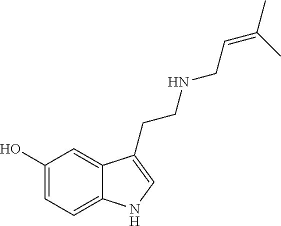 [SMILES: CC(C)=CCNCCc1c[nH]c2ccc(O)cc12] | (002) |
| 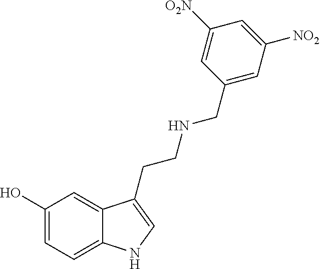 [SMILES: O=[N+]([O-])c1cc(CNCCc2c[nH]c3ccc(O)cc23)cc([N+](=O)[O-])c1] | (003) |
| 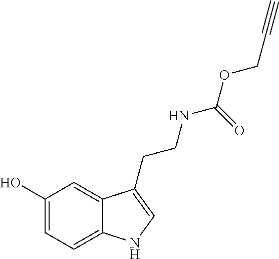 [SMILES: C#CCOC(=O)NCCc1c[nH]c2ccc(O)cc12] | (004) |
| 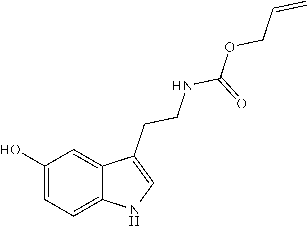 [SMILES: C=CCOC(=O)NCCc1c[nH]c2ccc(O)cc12] | (005) |
| 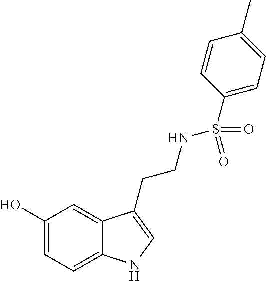 [SMILES: Cc1ccc(S(=O)(=O)NCCc2c[nH]c3ccc(O)cc23)cc1] | (006) |
| 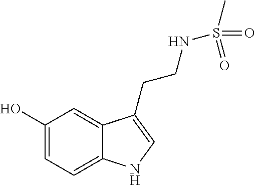 [SMILES: CS(=O)(=O)NCCc1c[nH]c2ccc(O)cc12] | (007) |
| 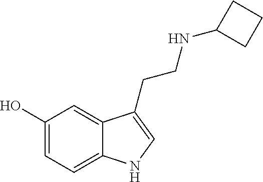 [SMILES: Oc1ccc2[nH]cc(CCNC3CCC3)c2c1] | (008) |
| 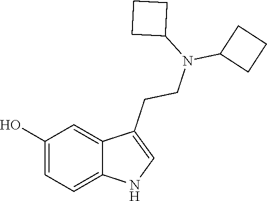 [SMILES: Oc1ccc2[nH]cc(CCN(C3CCC3)C3CCC3)c2c1] | (009) |
| 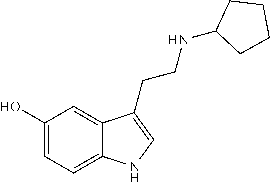 [SMILES: Oc1ccc2[nH]cc(CCNC3CCCC3)c2c1] | (010) |
| 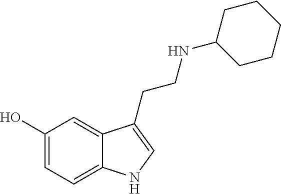 [SMILES: Oc1ccc2[nH]cc(CCNC3CCCCC3)c2c1] | (011) |
| 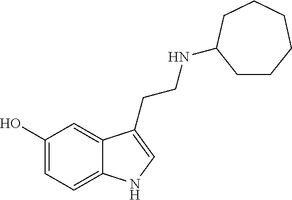 [SMILES: Oc1ccc2[nH]cc(CCNC3CCCCCC3)c2c1] | (012) |
| 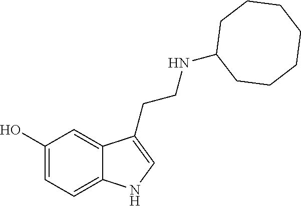 [SMILES: Oc1ccc2[nH]cc(CCNC3CCCCCCC3)c2c1] | (013) |
| 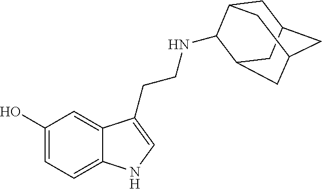 [SMILES: Oc1ccc2[nH]cc(CCNC3C4CC5CC(C4)CC3C5)c2c1] | (014) |
| 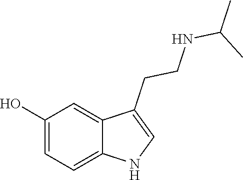 [SMILES: CC(C)NCCc1c[nH]c2ccc(O)cc12] | (015) |
| 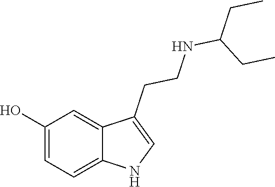 [SMILES: CCC(CC)NCCc1c[nH]c2ccc(O)cc12] | (016) |
| 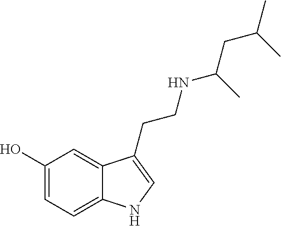 [SMILES: CC(C)CC(C)NCCc1c[nH]c2ccc(O)cc12] | (017) |
| 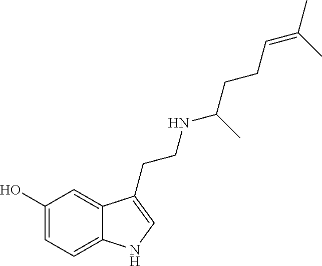 [SMILES: CC(C)=CCCC(C)NCCc1c[nH]c2ccc(O)cc12] | (018) |
| 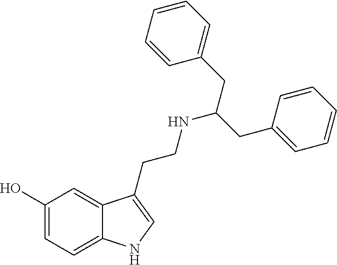 [SMILES: Oc1ccc2[nH]cc(CCNC(Cc3ccccc3)Cc3ccccc3)c2c1] | (019) |
| 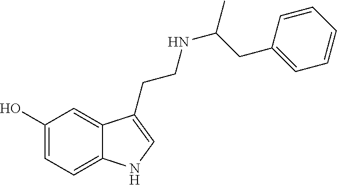 [SMILES: CC(Cc1ccccc1)NCCc1c[nH]c2ccc(O)cc12] | (020) |
| 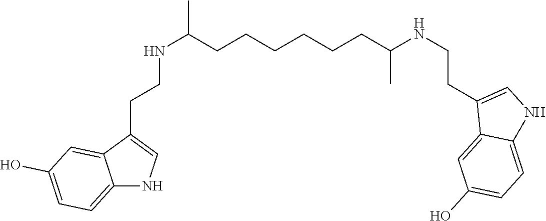 [SMILES: CC(CCCCCCC(C)NCCc1c[nH]c2ccc(O)cc12)NCCc1c[nH]c2ccc(O)cc12] | (021) |
| 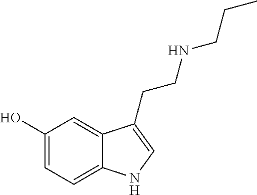 [SMILES: CCCNCCc1c[nH]c2ccc(O)cc12] | (022) |
| 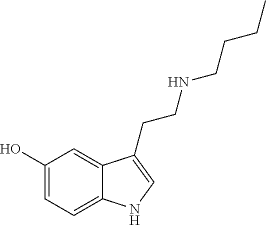 [SMILES: CCCCNCCc1c[nH]c2ccc(O)cc12] | (023) |
| 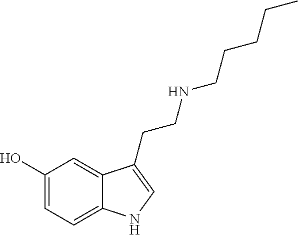 [SMILES: CCCCCNCCc1c[nH]c2ccc(O)cc12] | (024) |
| 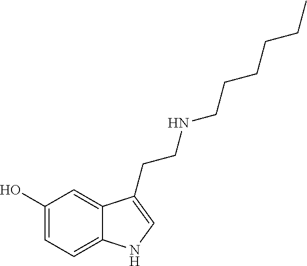 [SMILES: CCCCCCNCCc1c[nH]c2ccc(O)cc12] | (025) |
| 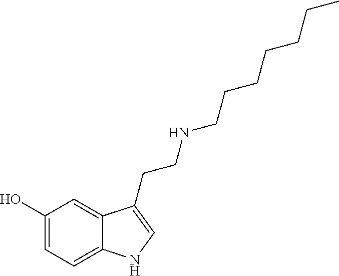 [SMILES: CCCCCCCNCCc1c[nH]c2ccc(O)cc12] | (026) |
| 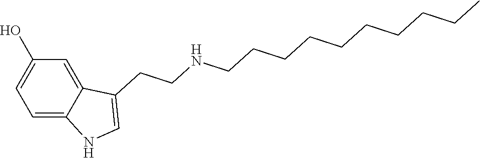 [SMILES: CCCCCCCCCCNCCc1c[nH]c2ccc(O)cc12] | (027) |
| 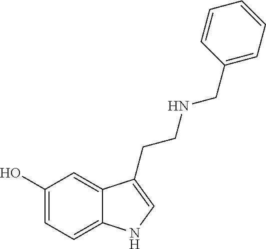 [SMILES: Oc1ccc2[nH]cc(CCNCc3ccccc3)c2c1] | (028) |
| 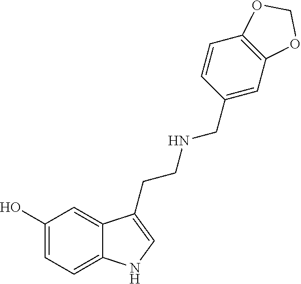 [SMILES: Oc1ccc2[nH]cc(CCNCc3ccc4c(c3)OCO4)c2c1] | (029) |
| 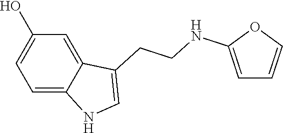 [SMILES: Oc1ccc2[nH]cc(CCNc3ccco3)c2c1] | (030) |
| 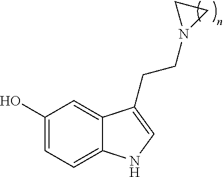 | (031) |
| n being an integer from 1 to 12 | |
| 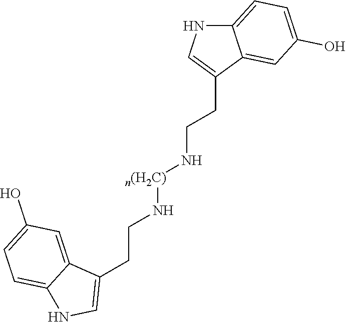 | (032) |
| n being an integer from 1 to 12 | |
| 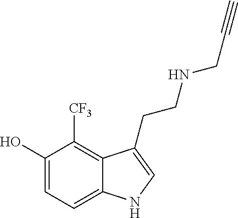 [SMILES: C#CCNCCc1c[nH]c2ccc(O)c(C(F)(F)F)c12] | (033) |
| 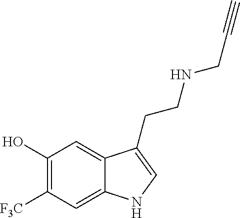 [SMILES: C#CCNCCc1c[nH]c2cc(C(F)(F)F)c(O)cc12] | (033)bis |
| 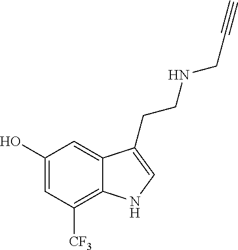 [SMILES: C#CCNCCc1c[nH]c2c(C(F)(F)F)cc(O)cc12] | (033ter) |
| 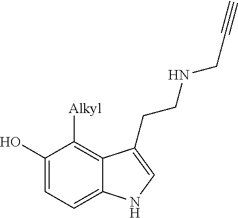 | (034) |
| Alkyl being (C₁-C₁₂)alkyl | |
| 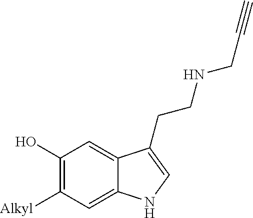 | (034)bis |
| Alkyl being (C₁-C₁₂)alkyl | |
| 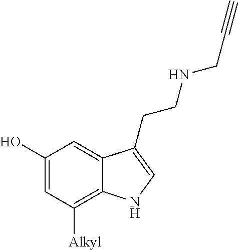 | (034)ter |
| Alkyl being (C₁-C₁₂)alkyl | |
| 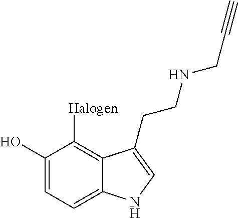 | (035) |
| Halogen being Cl or Br or I | |
| 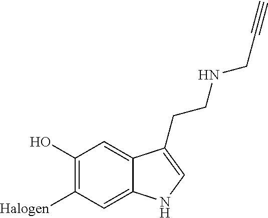 | (035bis) |
| Halogen being Cl or Br or I | |
| 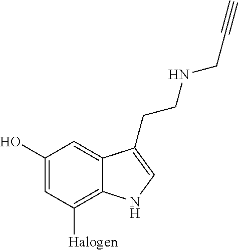 | (035ter) |
| Halogen being Cl or Br or I | |
| 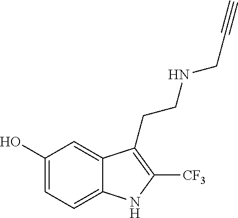 [SMILES: C#CCNCCc1c(C(F)(F)F)[nH]c2ccc(O)cc12] | (036) |
| 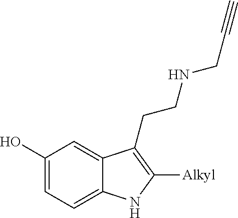 | (037) |
| Alkyl being (C₁-C₁₂)alkyl | |
| 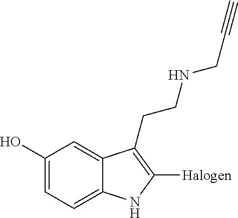 | (038) |
| Halogen being Cl or Br or I | |
| 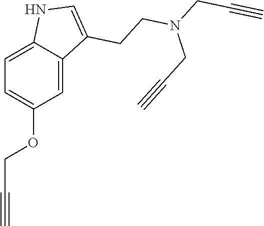 [SMILES: C#CCOc1ccc2[nH]cc(CCN(CC#C)CC#C)c2c1] | (A1) |
| 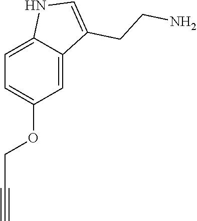 [SMILES: C#CCOc1ccc2[nH]cc(CCN)c2c1] | (A2) |
| 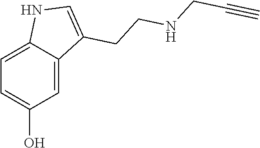 [SMILES: C#CCNCCc1c[nH]c2ccc(O)cc12] | (A3) |
| 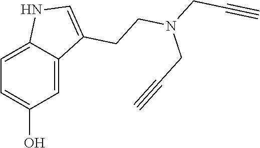 [SMILES: C#CCN(CC#C)CCc1c[nH]c2ccc(O)cc12] | (A4) |
| 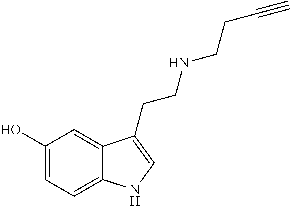 [SMILES: C#CCCNCCc1c[nH]c2ccc(O)cc12] | (039) |
| LC419 | |
| 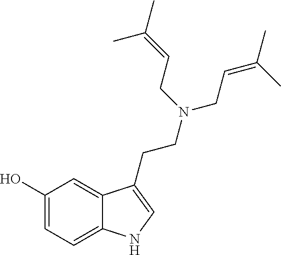 [SMILES: CC(C)=CCN(CC=C(C)C)CCc1c[nH]c2ccc(O)cc12] | (040) |
| LC342 | |
| 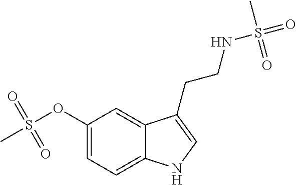 [SMILES: CS(=O)(=O)NCCc1c[nH]c2ccc(OS(C)(=O)=O)cc12] | (041) |
| LYS9 | |
| 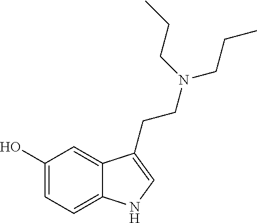 [SMILES: CCCN(CCC)CCc1c[nH]c2ccc(O)cc12] | (042) |
| 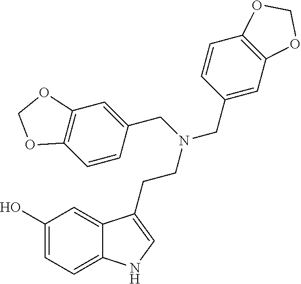 [SMILES: Oc1ccc2[nH]cc(CCN(Cc3ccc4c(c3)OCO4)Cc3ccc4c(c3)OCO4)c2c1] | (043) |
| LC353A | |
| 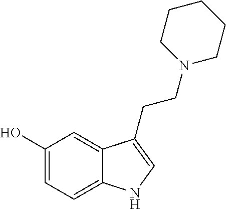 [SMILES: Oc1ccc2[nH]cc(CCN3CCCCC3)c2c1] | (045) |
| LC432F2 | |
| 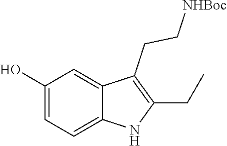 [SMILES: CCc1[nH]c2ccc(O)cc2c1CCNC(=O)OC(C)(C)C] | (046) |
| LC468 | |
| 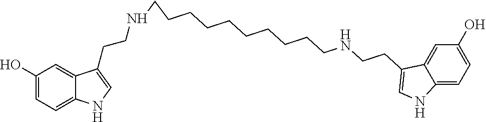 [SMILES: Oc1ccc2[nH]cc(CCNCCCCCCCCCCNCCc3c[nH]c4ccc(O)cc34)c2c1] | (047) |
| LC469 | |
| 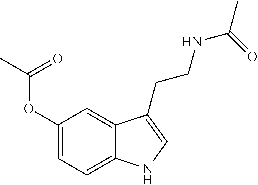 [SMILES: CC(=O)NCCc1c[nH]c2ccc(OC(C)=O)cc12] | (048) |
| LC377 | |
| 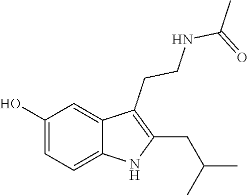 [SMILES: CC(=O)NCCc1c(CC(C)C)[nH]c2ccc(O)cc12] | (049) |
| LC385 | |
| 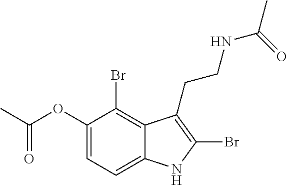 [SMILES: CC(=O)NCCc1c(Br)[nH]c2ccc(OC(C)=O)c(Br)c12] | (051) |
| LC380F2 | |
| 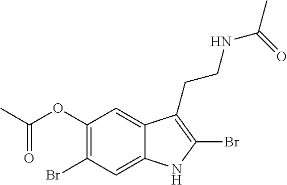 [SMILES: CC(=O)NCCc1c(Br)[nH]c2cc(Br)c(OC(C)=O)cc12] | (052) |
| LC380F3 | |
| 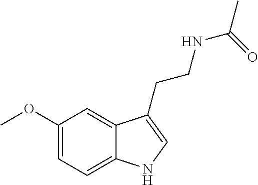 [SMILES: COc1ccc2[nH]cc(CCNC(C)=O)c2c1] | (054) |
| LC461 = Melatonin | |
| 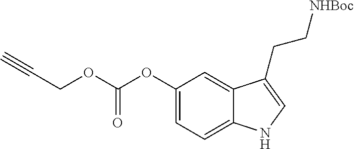 [SMILES: C#CCOC(=O)Oc1ccc2[nH]cc(CCNC(=O)OC(C)(C)C)c2c1] | (055) |
| LC483 | |
| 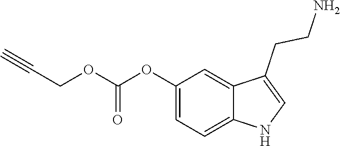 [SMILES: C#CCOC(=O)Oc1ccc2[nH]cc(CCN)c2c1] | (056) |
| LC487 | |
| 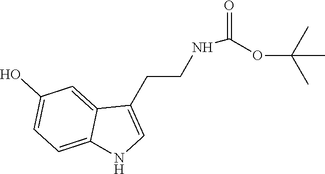 [SMILES: CC(C)(C)OC(=O)NCCc1c[nH]c2ccc(O)cc12] | (057) |
| LT273 | |
| 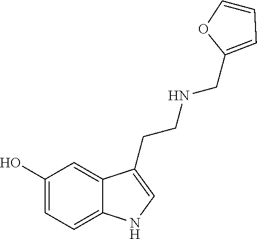 [SMILES: Oc1ccc2[nH]cc(CCNCc3ccco3)c2c1] | (058) |
| LT262B | |
| 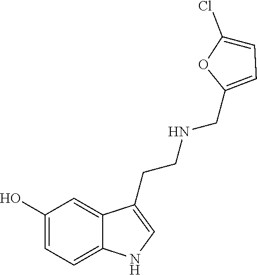 [SMILES: Oc1ccc2[nH]cc(CCNCc3ccc(Cl)o3)c2c1] | (059) |
| LT267C | |
| 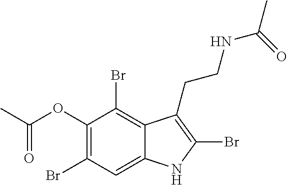 [SMILES: CC(=O)NCCc1c(Br)[nH]c2cc(Br)c(OC(C)=O)c(Br)c12] | (060) |
| LC380F4 | |
| 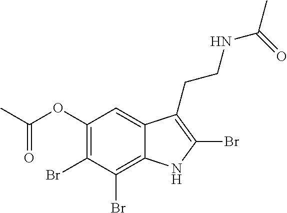 [SMILES: CC(=O)NCCc1c(Br)[nH]c2c(Br)c(Br)c(OC(C)=O)cc12] | (060a) |
| LC380F4′ | |
| 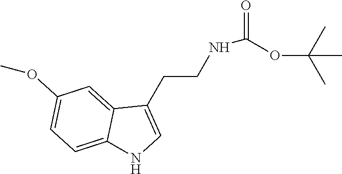 [SMILES: COc1ccc2[nH]cc(CCNC(=O)OC(C)(C)C)c2c1] | (061) |
| LT282 | |
| 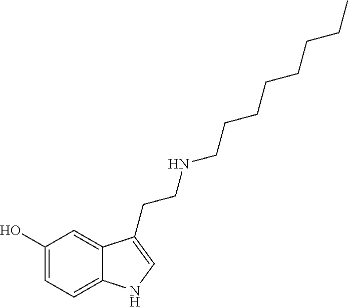 [SMILES: CCCCCCCCNCCc1c[nH]c2ccc(O)cc12] | (063) |
| LT266B | |
| 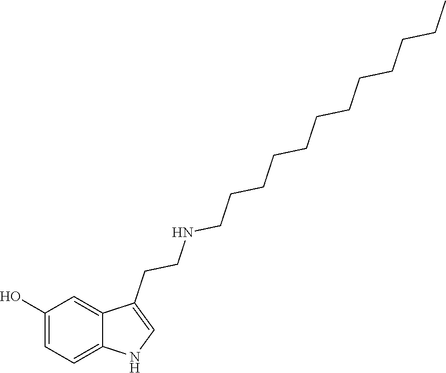 [SMILES: CCCCCCCCCCCCNCCc1c[nH]c2ccc(O)cc12] | (064) |
| LT266C | |

### Example 1: Synthesis of Compounds

### 1—General Information

All solvents and chemicals were purchased from commercially available sources and used without further purification or purified according to Purification of Laboratory Chemicals (Armarego, W.L.F.; Chai, C.L.L. 5^{th} Ed.). Solvents were dried under standard conditions. Reactions were monitored by thin layer chromatography (TLC) using pre-coated silica on aluminum plates from Merck (60_{F254}). TLC plates were visualized with UV-light and/or by treatment with ceric ammonium molybdate solution (CAM) and heating. Products were purified on column chromatography with Silica gel 60 from Macherey Nagel (0.036-0.071 mm; 215-400 mesh), a CombiFlash Rf+ Teledyne Isco system fitted with pre-packed silica gel columns (Interchim) or/and preparative HPLC Quaternary Gradient 2545 equipped with a Photodiode Array detector (Waters) fitted with a reverse phase column (XBridge Prep C18 5 μm OBD, 30×150 mm).

NMR spectroscopy was performed on Bruker spectrometers. Spectra were run in DMSO-d₆ or D₂O or CD₃OD, CD₂Cl₂ at 298 K. ¹H NMR were recorded at 400 or 500 MHz, and chemical shifts δ are expressed in ppm using the residual non-deuterated solvent signal as internal standard and the coupling constants J are specified in Hz. The following abbreviations are used: s, singlet; brs, broad singlet; d, doublet; dd, doublet of doublets; dt, doublet of triplets; dq, doublet of quartets; ddd, doublet of doublet of doublets; dqd, doublet of quartet of doublets; t, triplet; td, triplet of doublets; tdd, triplet of doublet of doublets; q, quartet; m, multiplet. We only reported labile protons that could be clearly identified in the spectra. ¹³C NMR were recorded at 101 or 126 MHZ, and chemical shifts δ are expressed in ppm using deuterated solvent signal as internal standard.

The purity of final compounds, determined to be >95% by UPLC MS, was recorded on a Waters Acquity H-class equipped with a Photodiode array detector and SQ Detector 2 with a reverse phase column (Aquity UPLC® BEH C18 1.7 μm, 2.1×50 mm).

“Classic System”: ACN (+0.1% FA) and MilliQ Water (+0.1% FA): isocratic at 5% of ACN (0.2 min), then linear gradient from 5% to 100% of ACN in 2.3 min, then isocratic at 100% of ACN (0.5 min).

### 2—Abbreviations

ACN, acetonitrile; AcOH, glacial acetic acid; aq., aqueous; Boc₂O, di-tert-butyl dicarbonate; DCM, dichloromethane; equiv., equivalent(s); ESI, electrospray ionization; EtOAc, ethyl acetate; EtOH, ethanol; Et₂O, diethyl ether; Et₃N, trimethylamine; FA, formic acid; HPLC, high pressure liquid chromatography; HRMS, high resolution mass spectroscopy; K₂CO₃, potassium carbonate; MeOH, methanol; MgSO₄, sulfate magnesium; MS, mass spectrometry; NaH, sodium hydride; NMR, nuclear magnetic resonance; RT, room temperature; THF, tetrahydrofuran; TLC, thin-layer chromatography; UPLC, ultra-high performance liquid chromatography; UV, ultraviolet.

### 3—Chemical Synthesis

### 3-1—Synthesis by Nucleophilic Substitution

### 3-1-1 Procedure for NH-Monoalkylated Compounds

In the dark and under inert atmosphere, serotonin hydrochloride (200 mg, 0.940 mmol, 1 equiv.) was dissolved in THF (10 mL). NaH (45 mg, 1.128 mmol, 1.2 equiv.) was added to the mixture. The mixture is stirred for 30 min, then alkyl bromide (0.6 equiv.) was added. The mixture was stirred for 3.5 h, then was quenched with water. The resulting solution was extracted with DCM, dried on MgSO₄ and concentrated. The crude was purified by flash chromatography using DCM/MeOH (100/0 to 80/20) as eluent and by preparative HPLC to give the desired product after lyophilization. The obtained compound is the formic salt.

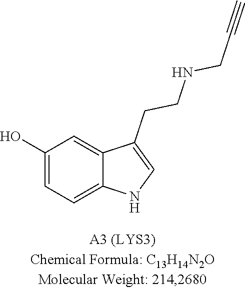 [SMILES: C#CCNCCc1c[nH]c2ccc(O)cc12]

Yield: 44 mg, 21%. Isolated as a light grey powder, >95% pure by NMR and a single spot by TLC; R_{f}: 0.44 in DCM/MeOH, 90/10. Strain green with CAM. ¹H NMR (DMSO-d₆, 500 MHz): δ ppm=10.51 (1H, s); 7.12 (1H, d, J=8.6 Hz); 7.04 (1H, d, J=2.4 Hz); 6.82 (1H, d, J=2.4 Hz); 6.60 (1H, dd, J=2.3, J=8.6 Hz); 3.52 (2H, d, J=2.4 Hz); 3.21 (1H, t, J=2.4 Hz); 2.92 (2H, m); 2.79 (2H, m). 13C NMR (DMSO-d₆, 126 MHz): δ ppm=150.2; 130.8; 127.8; 123.2; 111.7; 111.3; 110.5; 102.2; 80.8; 75.2; 47.9; 36.7; 24.2.UPLC: R_{T}: 0.8 to 1.0 (classic system). MS (ESI⁺) m/z [M+H]⁺ Calcd for C₁₃H₁₅N₂O⁺ 215.11; Found 215.22.

### LC419 (=Compound (039))

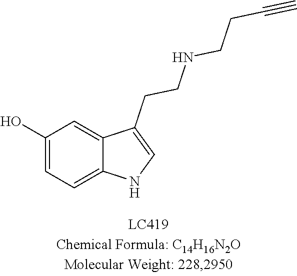 [SMILES: C#CCCNCCc1c[nH]c2ccc(O)cc12]

Yield: 28 mg, 12%. Isolated as a light grey powder, >95% pure by NMR and a single spot by TLC; R_{f}: 0.56 in DCM/MeOH, 80/20. Strain green with CAM;

¹H NMR (CD₃OD, 500 MHZ): δ ppm=8.51 (1H, s, FA); 7.19 (1H, d, J=8.8 Hz); 7.11 (1H, s); 6.93 (1H, d, J=2.1 Hz); 6.70 (1H, dd, J=2.2, J=8.8 Hz); 3.32-3.29 (2H, m); 3.18 (2H, t, J=7.4 Hz); 3.09 (2H, t, J=7.5 Hz); 2.62 (2H, dt, J=2.7 Hz, J=6.8 Hz); 2.52 (1H, t, J=2.6 Hz).

¹³C NMR (CD₃OD, 126 MHZ): δ ppm=169.8 (FA); 151.5; 133.2; 128.8; 124.9; 113.0; 112.9; 109.3; 103.1; 79.4; 73.1; 49.7; 47.1; 23.4; 16.9.

UPLC: R_{T}: 0.74 (classic system)

MS (ESI⁺) m/z [M+H]⁺ Calcd for C₁₄H₁₇N₂O⁺ 229.13; Found 229.28.

### LYS6 (LC344) (=Compound (002))

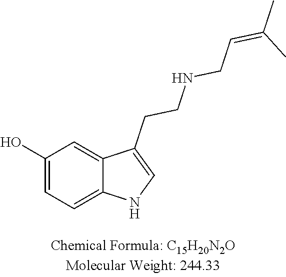 [SMILES: CC(C)=CCNCCc1c[nH]c2ccc(O)cc12]

Yield: 43 mg, 19%. Isolated as a light grey powder, >95% pure by NMR and a single spot by TLC; R_{f}: 0.5 in DCM/MeOH/Et₃N, 95/5/2. Strain green with CAM. ¹H NMR (CD₃OD, 400 MHZ): δ ppm=7.16 (1H, d, J=8.4 Hz); 7.00 (1H, s); 6.92 (1H, d, J=2.1 Hz); 6.67 (1H, dd, J=2.3 Hz, J=8.4 Hz); 5.21 (1H, m); 3.23 (2H, d, J=7.2 Hz); 2.89 (4H, s); 1.71 (3H, brs); 1.62 (3H, brs). ¹³C NMR (CD₃OD, 101 MHz): δ ppm=151.2; 136.8; 133.2; 129.3; 124.3; 122.3; 112.7; 112.5; 112.4; 103.5; 50.0; 47.4; 26.0; 25.9; 17.9. UPLC: R_{T}: 1.45 (classic system)

MS (ESI⁺) m/z [M+H]⁺ Calcd for C₁₅H₂₁N₂O⁺ 245.16; Found 245.17.

### LC342 (=Compound 040)

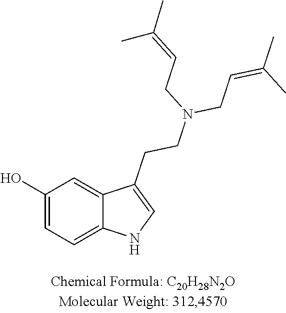 [SMILES: CC(C)=CCN(CC=C(C)C)CCc1c[nH]c2ccc(O)cc12]

Yield: 24 mg, 10%. Isolated as a light grey powder, >95% pure by NMR and a single spot by TLC; R_{f}: 0.49 in DCM/MeOH/Et₃N, 90/10/2. Strain green with CAM;

¹H NMR (CD₃OD, 400 MHZ): δ ppm=7.16 (1H, dd, J=0.5 Hz, J=8.5 Hz); 7.08 (1H, s); 6.85 (1H, dd, J=0.5 Hz, J=2.3 Hz); 6.67 (1H, dd, J=2.4 Hz, J=8.7 Hz); 5.29 (2H, m); 3.72 (4H, d, J=7.5 Hz); 3.27 (2H, m); 3.08 (2H, m); 1.81 (6H, brs); 1.72 (6H, d, J=0.9 Hz).

¹³C NMR (CD₃OD, 101 MHZ): δ ppm=151.6; 145.3 (2C); 133.2; 128.8; 125.0 (2C); 114.3; 113.1; 112.9; 109.2; 103.2; 53.3; 51.9 (2C); 26.2 (2C); 22.0; 18.5 (2C).

UPLC: R_{T}: 1.81 (classic system)

MS (ESI⁺) m/z [M+H]⁺ Calcd for C₂₀H₂₉N₂O⁺ 313.22; Found 313.04.

### LYS7 (LC330) (=Compound (003))

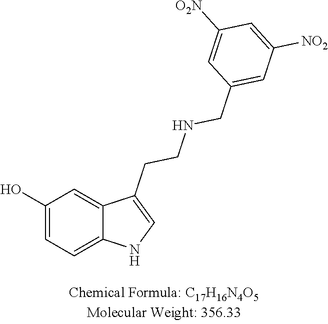 [SMILES: O=[N+]([O-])c1cc(CNCCc2c[nH]c3ccc(O)cc23)cc([N+](=O)[O-])c1]

Yield: 124 mg, 37%. Isolated as a orange powder, >95% pure by NMR and a single spot by TLC; R_{f}: 0.48 in DCM/MeOH, 90/10. Strain green with CAM;

¹H NMR (DMSO-d₆, 400 MHZ): δ ppm=10.43 (1H, brs); 8.68 (1H, t, J=2.2 Hz); 8.60 (2H, d, J=2.1 Hz); 8.51 (1H, s); 7.08 (1H, d, J=8.5 Hz); 7.01 (1H, d, J=2.5 Hz); 6.74 (1H, d, J=2.2 Hz); 6.55 (1H, dd, J=2.3 Hz, J=8.6 Hz); 3.98 (2H, s); 2.77 (4H, m).

¹³C NMR (DMSO-d₆, 101 MHZ): δ ppm=150.5; 148.4 (2C); 146.9; 131.3; 128.5 (2C); 128.3; 123.5; 117.2; 112.0; 111.8; 111.6; 102.6; 51.7; 49.6; 26.2.

UPLC: R_{T}: 1.48 (classic system)

MS (ESI⁺) m/z [M+H]⁺ Calcd for C₁₇H₁₇N₄O₅⁺ 357.11; Found 357.13.

### 3-1-2—Procedure for Polyalkylated Compounds

In the dark and under inert atmosphere, serotonin hydrochloride (500 mg, 2.35 mmol, 1 equiv.) was dissolved in THF (25 mL). NaH (188 mg, 2 equiv.) was added to the mixture. The mixture is stirred for 30 min, and then propargyl bromide (524 μL, 2 equiv.) was added. The mixture was stirred for 4 h, then was quenched with water. The resulting solution was extracted with DCM, dried on MgSO₄ and concentrated. The crude was purified by flash chromatography using DCM/MeOH (99/1 to 80/20) as eluent. 3 fractions were obtained and purified by preparative HPLC to give 3 products after lyophilization.

### A1 (LYS1) (NMR-SD095A1):

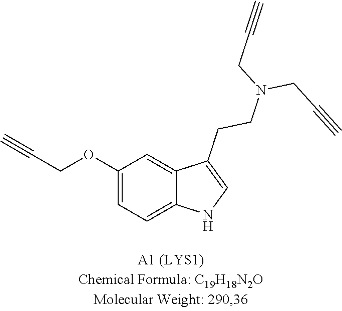 [SMILES: C#CCOc1ccc2[nH]cc(CCN(CC#C)CC#C)c2c1]

Yield: 5.5 mg, 0.8%. Isolated as a light grey powder, >95% pure by NMR and a single spot by TLC; R_{f}: 0.71 in DCM/MeOH, 95/5. Strain green with CAM, ¹H NMR (CD₃OD, 500 MHZ): δ ppm=7.23 (1H, d, J=8.8 Hz); 7.16 (1H, d, J=2.3 Hz); 7.06 (1H, s); 6.80 (1H, dd, J=2.3 Hz, J=8.8 Hz); 4.72 (2H, d, J=2.4 Hz); 3.56 (4H, d, J=2.5 Hz); 2.90 (4H, m); 2.89 (1H, t, J=2.4 Hz); 2.69 (2H, t, J=2.5 Hz). ¹³C NMR (CD₃OD, 126 MHZ): δ ppm=152.9; 133.9; 128.9; 124.2; 113.3 (2C); 112.9; 103.6; 80.7; 79.0 (2C); 76.2; 75.1 (2C); 57.8; 54.7; 42.8 (2C); 24.2. UPLC: R_{T}: 1.88 (classic system). MS (ESI⁺) m/z [M+H]⁺ Calcd for C₁₉H₁₉N₂O⁺ 291.14; Found 291.18.

A3 (LYS3) (NMR-SD095A3-Formic acid salt):

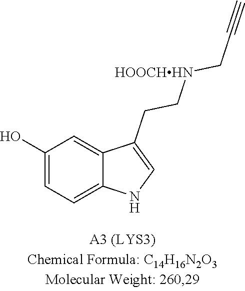

Yield: 29 mg, 6%. Isolated as a light grey powder, >95% pure by NMR and a single spot by TLC; R_{f}: 0.14 in DCM/MeOH, 95/5. Strain green with CAM. ¹H NMR (DMSO-d⁶, 500 MHZ): δ ppm=10.51 (1H, s); 7.12 (1H, d, J=8.6 Hz); 7.04 (1H, d, J=2.4 Hz); 6.82 (1H, d, J=2.4 Hz); 6.60 (1H, dd, J=2.3, J=8.6 Hz); 3.52 (2H, d, J=2.4 Hz); 3.21 (1H, t, J=2.4 Hz); 2.92 (2H, m); 2.79 (2H, m). ¹³C NMR (DMSO-d₆, 126 MHZ): δ ppm=150.2; 130.8; 127.8; 123.2; 111.7; 111.3; 110.5; 102.2; 80.8; 75.2; 47.9; 36.7; 24.2. UPLC: R_{T}: 0.8 to 1.0 (classic system). MS (ESI⁺) m/z [M+H−HCOOH]⁺ Calcd for C₁₃H₁₅N₂O⁺ 215.11; Found 215.22.

### A4 (LYS4) (NMR-SD095A4):

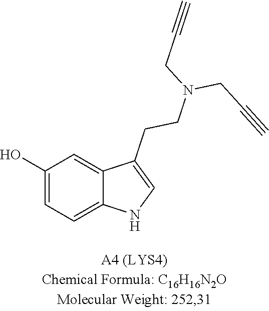 [SMILES: C#CCN(CC#C)CCc1c[nH]c2ccc(O)cc12]

Yield: 57 mg, 10%. Isolated as a light grey powder, >95% pure by NMR and a single spot by TLC; R_{f}: 0.43 in DCM/MeOH, 95/5. Strain green with CAM. ¹H NMR (CD₃OD, 500 MHZ): δ ppm=7.15 (1H, d, J=8.3 Hz); 6.99 (1H, s); 6.95 (1H, d, J=2.3 Hz); 6.67 (1H, dd, J=2.3 Hz, J=8.6 Hz); 3.55 (4H, d, J=2.5 Hz); 2.90-2.85 (4H, m); 2.70 (2H, t, J=2.5 Hz). ¹³C NMR (CD₃OD, 126 MHz): δ ppm=151.0; 133.1; 129.3; 1234.1; 112.7; 112.5; 112.4; 103.6; 78.7 (2C); 75.3 (2C); 54.7; 42.8 (2C); 24.2. UPLC: R_{T}: 1.42 (classic system). MS (ESI⁺) m/z [M+H]⁺ Calcd for C₁₆H₁₇N₂O⁺ 253.13; Found 253.15.

### A0 (LC431F1):

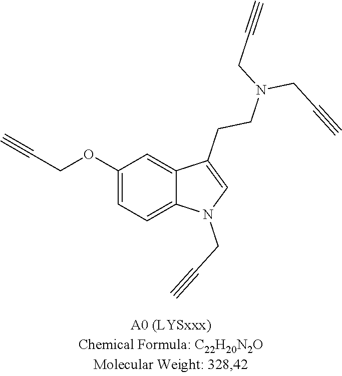 [SMILES: C#CCOc1ccc2c(c1)c(CCN(CC#C)CC#C)cn2CC#C]

Yield: 67 mg, 9%. Isolated as a light grey powder, >95% pure by NMR and a single spot by TLC; R_{f}: 0.68 in DCM/MeOH, 98/2. Strain green with CAM;

¹H NMR (CD₂Cl₂, 500 MHz): δ ppm=7.29 (1H, d, J=8.8 Hz); 7.16 (1H, d, J=2.5 Hz); 7.06 (1H, s); 6.92 (1H, dd, J=2.4 Hz, J=8.8 Hz); 4.82 (2H, d, J=2.4 Hz); 4.74 (2H, d, J=2.5 Hz); 3.52 (4H, d, J=2.5 Hz); 2.90-2.85 (4H, m); 2.56 (1H, t, J=2.5 Hz); 2.44 (1H, t, J=2.5 Hz); 2.30 (2H, t, J=2.5 Hz).

¹³C NMR (CD₂Cl₂, 126 MHZ): δ ppm=151.9; 131.9; 128.7; 125.9; 113.3; 112.5; 110.2; 103.4; 79.3; 78.9 (2C); 78.1; 74.8; 72.9; 72.7 (2C); 56.8; 53.3; 42.1 (2C); 35.8; 23.4.

UPLC: R^{T}: 2.02 (classic system)

MS (ESI⁺) m/z [M+H]⁺ Calcd for C₂₂H₂₁N₂O⁺ 329.16; Found 329.20.

### 3-1-3—Procedure for O-Alkylated Compounds

In the dark, serotonin hydrochloride (500 mg, 2.35 mmol, 1 equiv.) was dissolved in water (9 mL). K₂CO₃ (665 mg, 4.81 mmol, 2.1 equiv.) and Boc₂O (538 mg, 2.46 mmol, 1.05 equiv.) were added to the solution. The solution was stirred overnight, and then extracted with DCM. The organic phase was washed with aq. HCl 5% and brine, then dried on MgSO₄ and concentrated. The crude was purified by flash chromatography using DCM/MeOH (100/0 to 90/10) as eluent to give the desired product (485 mg), which was engaged in the next step.

In the dark and under inert atmosphere, the product of the previous step (138 mg, 0.5 mmol, 1 equiv.) was dissolved in dry acetonitrile (4 mL). K₂CO₃ (138 mg, 2 equiv.) and propargyl bromide (59 μL, 1.06 equiv.) were added to the solution. The mixture was stirred and heated at reflux overnight, cooled down to r.t. then filtered with acetonitrile and concentrated. The crude was purified by flash chromatography using cyclohexane/EtOAC (90/10 to 0/100) as eluent to give LC466.

The product was then dissolved in CH₂Cl₂/TFA, 4/1 (4 mL) and stirred for 2 h until the product was predominant in UPLC analysis. The mixture was then concentrated, and the crude was directly purified by preparative HPLC to give the desired product after lyophilization.

### A2 (LYS2) (NMR-SD08801-Formic Acid Salt):

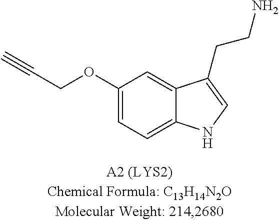 [SMILES: C#CCOc1ccc2[nH]cc(CCN)c2c1]

Yield: 39 mg, 17%. Isolated as a light grey powder, >95% pure by NMR and a single spot by TLC; R_{f}: 0.68 in DCM/MeOH/Et₃N, 90/10/2. Strain green with CAM. ¹H NMR (D₂O, 500 MHZ): δ ppm=8.45 (1H, s, (FA)); 7.47 (1H, d, J=8.7 Hz); 7.31 (1H, s); 7.29 (1H, d, J=2.5 Hz); 7.00 (1H, dd, J=8.9 Hz, J=2.4 Hz); 4.84 (2H, d, J=2.4 Hz); 3.32 (2H, t, J=7.1 Hz); 3.14 (2H, t, J=7.1 Hz); 2.95 (1H, t, J=2.4 Hz). UPLC: R_{T}: 1.57 (classic system). MS (ESI⁺) m/z [M+H]⁺ Calcd for C₁₃H₁₅N₂O⁺215.11; Found 215.23.

### 3-1-4—Procedure for Carbamate Esters

In the dark and under inert atmosphere, serotonin hydrochloride (75 mg, 0.315 mmol, 1 equiv.) was dissolved in DCM/H₂O, ½ (¼ mL). Na₂CO₃ (36 mg, 0.740 mmol, 2.1 equiv.) was added to the mixture. The mixture is stirred for 30 min, and then alkylchloroformate (1 equiv.) was added. The mixture was stirred for 18 h, then was quenched with water. The resulting solution was extracted with DCM, dried on MgSO₄ and concentrated. The crude was purified by flash chromatography using heptane/EtOAc (100/0 to 50/50) as eluent and then purified by preparative HPLC.

### LYS8=LC406F2 (=Compound (004))

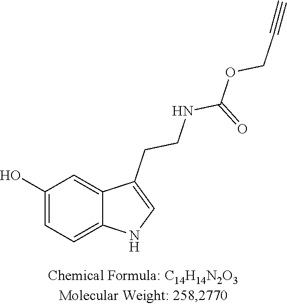 [SMILES: C#CCOC(=O)NCCc1c[nH]c2ccc(O)cc12]

Yield: 39 mg, 17%. Isolated as a light grey powder, >95% pure by NMR and a single spot by TLC; R_{f}: 0.68 in DCM/MeOH/Et₃N, 90/10/2. Strain green with CAM. ¹H NMR (CD₃OD, 500 MHZ): δ ppm=7.15 (1H, d, J=8.7 Hz); 6.98 (1H, s); 6.95 (1H, d, J=2.2 Hz); 6.67 (1H, dd, J=2.2 Hz, J=8.7 Hz); 4.63 (2H, d, J=2.2 Hz); 3.36 (2H, t, J=7.4 Hz); 2.85 (2H, t, J=7.4 Hz); 2.83 (1H, t, J=2.4 Hz). ¹³C NMR (CD₃OD, 126 MHz): δ ppm=157.9; 151.0; 133.0; 129.3; 124.2; 112.6; 112.3 (2C); 103.5; 79.5; 75.6; 53.0; 42.6; 26.7. UPLC: R_{T}: 1.83 (classic system). MS (ESI⁺) m/z [M+H]⁺ Calcd for C₁₄H₁₅N₂O⁺ 259.10; Found 259.24.

### 3-1-5—Procedure for Sulfonamides:

In the dark, serotonin hydrochloride (200 mg, 0.94 mmol) was dissolved in DCM (9 mL). Triethylamine (210 μL, 1.5 mmol, 1.6 equiv.) was added to the mixture. The mixture is stirred for 30 min, and then methanesulfonyl chloride (43 μL, 0.56 mmol, 0.6 equiv.) was added slowly. The mixture was stirred for 18 h, then was quenched with water. The resulting solution was extracted with DCM, dried on MgSO₄ and concentrated. The crude was purified by flash chromatography using n-Hexane/EtOAc (20/80 to 0/100) as eluent to afford a white solid.

### LYS9 (=Compound (041))

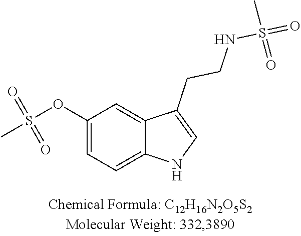 [SMILES: CS(=O)(=O)NCCc1c[nH]c2ccc(OS(C)(=O)=O)cc12]

Yield: 15 mg, 7%. Isolated as a pale yellow oil, >95% pure by NMR and a single spot by TLC; R_{f}: 0.23 in n-Hex/EtOAc, 10/90. Strain green with CAM;

¹H NMR (CD₃OD, 500 MHZ): δ ppm=7.54 (1H, d, J=2.2 Hz); 7.39 (1H, d, J=8.7 Hz); 7.25 (1H, s); 7.07 (1H, dd, J=2.3 Hz, J=8.6 Hz); 3.36 (2H, t, J=7.2 Hz); 3.18 (3H, s); 2.99 (2H, t, J=7.2 Hz); 2.81 (3H, s).

¹³C NMR (CD₃OD, 126 MHz): δ ppm=144.3; 136.6; 128.9; 126.2; 116.8; 113.7; 113.1; 112.5; 44.9; 39.9; 36.9; 27.3.

UPLC: R_{T}: 1.45 (classic system)

MS (ESI⁺) m/z [M+H]⁺ Calcd for C₁₂H₁₇N₂O₅S₂⁺ 333.05; Found 333.18.

### 3-1-6—Procedure for NH-Alkylated Tryptamine Derivatives

In the dark and under inert atmosphere, tryptamin (160 mg, 1 mmol, 1 equiv.) was dissolved in DMF (10 mL). K₂CO₃ (138 mg, 1 equiv.) was added to the mixture. The mixture is stirred for 5 min, then alkyl bromide (0.6 equiv.) was added. The mixture was stirred for 4 h, then was quenched with water. The resulting solution was extracted with DCM, dried on MgSO₄ and concentrated. The crude was purified by flash chromatography using DCM/MeOH (100/0 to 80/20) as eluent and by preparative HPLC to give the desired product after lyophilization. The obtained compound is the formic salt.

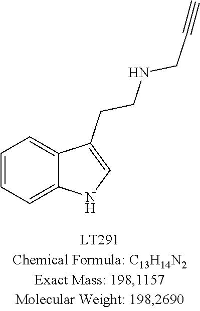

Yield: 25 mg, 13%. Isolated as a white powder, >95% pure by NMR and a single spot by TLC; R_{f}: 0.26 in DCM/MeOH, 95/5. Strain green with CAM;

¹H NMR (CD₃OD, 500 MHZ): δ ppm=8.51 (1H, s, FA); 7.58 (1H, td, J=0.9 Hz, J=7.9 Hz); 7.38 (1H, td, J=0.8 Hz, J=8.1 Hz); 7.17 (1H, brs); 7.13 (1H, dt, J=0.9 Hz, J=7.9 Hz); 7.05 (1H, dt, J=0.9 Hz, J=8.1 Hz); 3.87 (2H, d, J=2.5 Hz); 3.36 (2H, t, J=7.5 Hz); 3.15 (2H, t, J=7.4 Hz); 3.13 (1H, t, J=2.5 Hz).

¹³C NMR (CD₃OD, 126 MHZ): δ ppm=169.7 (FA); 138.3; 128.1; 124.2; 122.7; 120.0; 118.9; 112.5; 110.3; 78.4; 75.6; 48.4; 37.4; 23.5.

UPLC: R_{T}: 1.45 (classic system)

MS (ESI⁺) m/z [M+H]⁺ Calcd for C₁₃H₁₅N₂⁺ 199.12; Found 199.23.

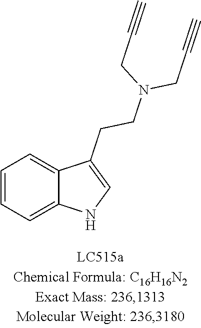 [SMILES: C#CCN(CC#C)CCc1c[nH]c2ccccc12]

Yield: 160 mg, 67%. Isolated as a white powder, >95% pure by NMR and a single spot by TLC; R_{f}: 0.63 in DCM/MeOH, 95/5. Strain green with CAM;

¹H NMR (CD₂Cl₂, 500 MHZ): δ ppm=8.11 (1H, brs); 8.09 (1H, s, FA); 7.61 (1H, dd, J=0.9 Hz, J=7.8 Hz); 7.36 (1H, td, J=0.9 Hz, J=8.2 Hz); 7.16 (1H, dt, J=1.0 Hz, J=7.9 Hz); 7.08 (2H, m); 3.53 (4H, d, J=2.5 Hz); 2.94 (2H, m); 2.89 (2H, m); 2.29 (2H, t, J=2.5 Hz).

¹³C NMR (CD₂Cl₂, 126 MHZ): δ ppm=136.7; 127.9; 122.2; 122.1; 119.5; 119.1; 114.2; 111.5; 79.2 (2C); 73.2 (2C); 53.8; 42.4 (2C); 23.8.

UPLC: R_{T}: 1.68 (classic system)

MS (ESI⁺) m/z [M+H]⁺ Calcd for C₁₆H₁₇N₂⁺ 237.13; Found 237.19.

### 3-2—Synthesis by Reductive Amination

3-2-1—Procedure from Ketone (Singly Substituted Analogs) [Compounds 1 to 7]:

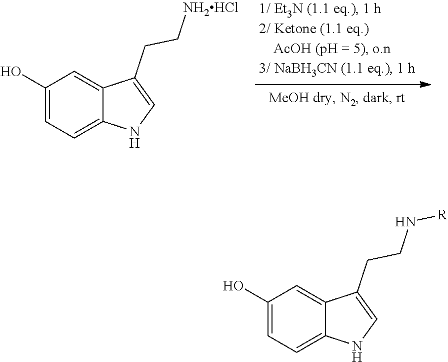

In the dark and under inert atmosphere, serotonin hydrochloride (213 mg, 1 mmol) was dissolved in dry MeOH (10 mL). Et₃N (153 μL, 1.1 equiv.) was added to the mixture. The mixture was stirred for 30 min at RT, and then the corresponding ketone (1.1 eq.) was added. The mixture was stirred overnight, and after that time NaBH₃CN (1.1 equiv.) was added. The reaction mixture was stirred further at RT for additional 60 minutes. Next, the solvent was evaporated under reduced pressure. The crude was taken in a mixture of Et₂O/water, 1/1 (10/10 mL), the resulting solution was alkalinized with NaOH [2M] until to obtain pH=10, then extracted with Et₂O (2×20 mL), then with DCM (1×20 mL). The organic phases were dried over MgSO₄ and concentrated. The crude was purified by flash chromatography using DCM/MeOH/Et₃N (100/0/2 to 80/20/2) as eluent.

### LYS12 Compound 1: LC332-333-338 ((=Compound (008))

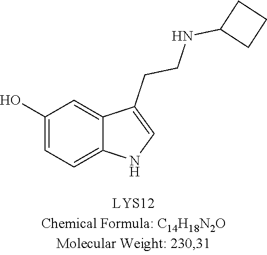 [SMILES: Oc1ccc2[nH]cc(CCNC3CCC3)c2c1]

Yield: 39 mg, 17%. Isolated as a light grey powder, >95% pure by NMR and a single spot by TLC; R_{f}: 0.68 in DCM/MeOH/Et₃N, 90/10/2. Strain green with CAM. ¹H NMR (CD₃OD, 400 MHZ): δ ppm=7.16 (1H, d, J=8 Hz); 7.00 (1H, s); 6.91 (1H, d, J=4 Hz); 6.67 (1H, dd, J=4 Hz, J=8 Hz); 3.33 (1H, m); 2.84 (4H, m); 2.20 (2H, m); 1.72 (4H, m). ¹³C NMR (CD₃OD, 101 MHZ): δ ppm=151.2; 133.2; 129.2; 124.3; 112.7; 112.5; 112.4; 103.4; 54.6; 47.7; 30.8 (2C); 26.1; 15.6. UPLC: R_{T}: 1.21 (classic system). MS (ESI⁺) m/z [M+H]⁺ Calcd for C₁₄H₁₉N₂O⁺ 231.14; Found 231.17.

### LC348 (=Compound (009))

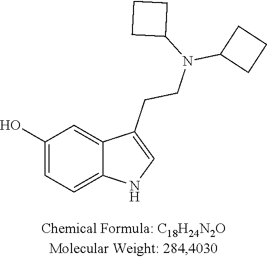 [SMILES: Oc1ccc2[nH]cc(CCN(C3CCC3)C3CCC3)c2c1]

Yield: 138 mg, 48%. Isolated as a light grey powder, >95% pure by NMR and a single spot by TLC; R_{f}: 0.62 in DCM/MeOH/Et₃N, 98/2/2. Strain green with CAM;

¹H NMR (CD₃OD, 400 MHZ): δ ppm=8.54 (1H, s, FA) 7.20 (1H, dd, J=0.5 Hz, J=8.6 Hz); 7.11 (1H, s); 6.88 (1H, dd, J=0.5 Hz, J=2.3 Hz); 6.71 (1H, dd, J=2.3 Hz, J=8.6 Hz); 3.88 (2H, quint, J=8.5 Hz); 3.19 (2H, m); 3.03 (2H, m); 2.33 (8H, m); 1.86 (4H, m).

¹³C NMR (CD₃OD, 101 MHZ): δ ppm=169.9 (FA); 151.6; 133.1; 128.7; 124.9; 113.1; 112.8; 109.6; 102.8; 58.2 (2C); 50.2; 28.9 (4C); 20.4; 15.7 (2C).

UPLC: R_{T}: 1.49 (classic system)

MS (ESI⁺) m/z [M+H]⁺ Calcd for C₁₈H₂₅N₂O⁺ 285.18 Found 285.34.

### LYS13 Compound 2: LC335 ((=Compound (010))

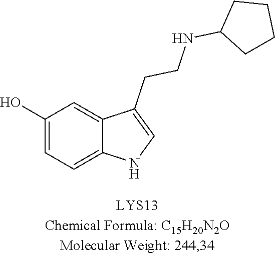 [SMILES: Oc1ccc2[nH]cc(CCNC3CCCC3)c2c1]

Yield: 195 mg, 80%. Isolated as a light grey powder, >95% pure by NMR and a single spot by TLC; R_{f}: 0.51 in DCM/MeOH/Et₃N, 95/5/2. Strain green with CAM. ¹H NMR (CD₃OD, 400 MHZ): δ ppm=7.16 (1H, d, J=8 Hz); 7.01 (1H, s); 6.92 (1H, d, J=4 Hz); 6.67 (1H, dd, J=4 Hz, J=8 Hz); 3.11 (1H, m); 2.90 (4H, s); 1.90 (2H, m); 1.66 (2H, m); 1.54 (2H, m); 1.36 (2H, m). ¹³C NMR (CD₃OD, 101 MHZ): δ ppm=151.2; 133.2; 129.3; 124.3; 112.7; 112.5 (2C); 103.5; 60.7; 49.6; 33.2 (2C); 26.1; 25.0 (2C). UPLC: R_{T}: 1.34 (classic system). MS (ESI⁺) m/z [M+H]⁺ Calcd for C₁₅H₂₁N₂O⁺245.16; Found 245.10.

### LYS14 Compound 3: LC336 (=Compound (011))

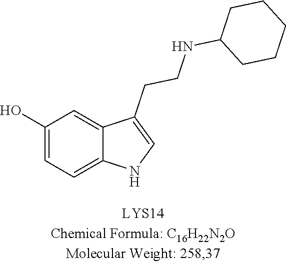 [SMILES: Oc1ccc2[nH]cc(CCNC3CCCCC3)c2c1]

Yield: 253 mg, 98%. Isolated as a light grey powder, >95% pure by NMR and a single spot by TLC; R_{f}: 0.53 in DCM/MeOH/Et₃N, 95/5/2. Strain green with CAM. ¹H NMR (CD₃OD, 400 MHZ): δ ppm=7.16 (1H, d, J=8 Hz); 7.01 (1H, s); 6.93 (1H, d, J=4 Hz); 6.67 (1H, dd, J=4 Hz, J=8 Hz); 2.90 (4H, m); 2.50 (1H, m); 1.90 (2H, m); 1.73 (2H, m); 1.63 (1H, m); 1.21 (5H, m). ¹³C NMR (CD₃OD, 101 MHZ): δ ppm=151.2; 133.2; 129.3; 124.3; 112.8; 112.5; 112.4; 103.5; 57.9; 47.6; 33.5 (2C); 27.1; 26.1 (3C). UPLC: R_{T}: 1.46 (classic system). MS (ESI⁺) m/z [M+H]⁺ Calcd for C₁₆H₂₃N₂O⁺ 259.17; Found 259.24.

### LYS15 Compound: LC356 (=Compound (012))

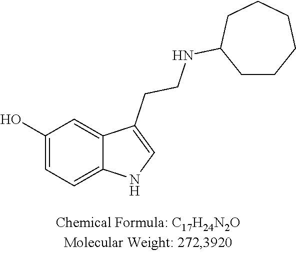 [SMILES: Oc1ccc2[nH]cc(CCNC3CCCCCC3)c2c1]

Yield: 120 mg, 44%. Isolated as a light grey powder, >95% pure by NMR and a single spot by TLC; R_{f}: 0.43 in DCM/MeOH/Et₃N, 95/5/2. Strain green with CMA. ¹H NMR (CD₃OD, 400 MHZ): δ ppm=7.17 (1H, d, J=8.6 Hz); 7.02 (1H, s); 6.93 (1H, d, J=2.5 Hz); 6.67 (1H, dd, J=2.4 Hz, J=8.6 Hz); 2.91 (4H, m); 2.71 (1H, m); 1.89-1.80 (2H, m); 1.71-1.32 (10H, m). ¹³C NMR (CD₃OD, 101 MHZ): δ ppm=151.2; 133.2; 129.2; 124.4; 112.8; 112.5; 112.2; 103.4; 60.2; 48.0; 34.8 (2C); 29.1 (2C); 25.8; 25.5 (2C).

UPLC: R_{T}: 1.57 (classic system)

MS (ESI⁺) m/z [M+H]⁺ Calcd for C₁₇H₂₅N₂O⁺ 273.19; Found 273.30.

### LYS16 Compound 4: LC337 (=Compound 013)

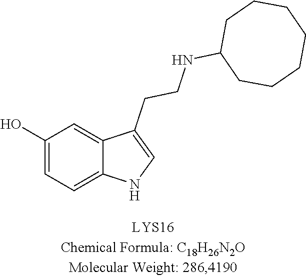 [SMILES: Oc1ccc2[nH]cc(CCNC3CCCCCCC3)c2c1]

Yield: 212 mg, 74%. Isolated as a light grey powder, >95% pure by NMR and a single spot by TLC; R_{f}: 0.6 in DCM/MeOH/Et₃N, 90/10/2. Strain green with CMA;

¹H NMR (CD₃OD, 400 MHZ): δ ppm=7.17 (1H, d, J=8 Hz); 7.02 (1H, s); 6.93 (1H, d, J=4 Hz); 6.67 (1H, dd, J=4 Hz, J=8 Hz); 2.94 (4H, m); 2.78 (1H, m); 1.73 (4H, m); 1.48 (10H, m).

¹³C NMR (CD₃OD, 101 MHZ): δ ppm=151.2; 133.2; 129.2; 124.4; 112.8; 112.6; 112.1; 103.4; 59.1; 47.9; 32.9 (2C); 27.9 (2C); 26.9; 25.8; 25.3 (2C).

UPLC: R_{T}: 1.66 (classic system)

MS (ESI⁺) m/z [M+H]⁺ Calcd for C₁₈H₂₇N₂O⁺ 287.20; Found 287.30.

### LYS17 Compound 5: LC341 (=Compound (0014))

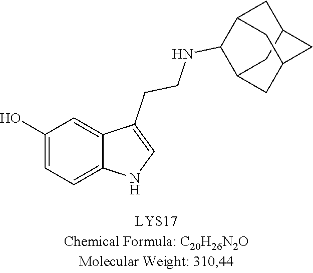 [SMILES: Oc1ccc2[nH]cc(CCNC3C4CC5CC(C4)CC3C5)c2c1]

Yield: 300 mg, 97%. Isolated as a a light grey powder, >95% pure by NMR and a single spot by TLC; R_{f}: 0.53 in DCM/MeOH/Et₃N, 95/5/2. Strain green with CMA;

¹H NMR (CD₃OD, 400 MHZ): δ ppm=7.17 (1H, d, J=8.6 Hz); 7.04 (1H, s); 6.94 (1H, d, J=2.3 Hz); 6.67 (1H, dd, J=2.4 Hz, J=8.6 Hz); 2.93 (4H, m); 2.79 (1H, brs); 1.91-1.83 (4H, m); 1.80 (1H, m); 1.74-1.68 (4H, m); 1.65-1.56 (3H, m); 1.47-1.41 (2H, m).

¹³C NMR (CD₃OD, 101 MHZ): δ ppm=151.2; 133.2; 129.3; 124.4; 112.8; 112.5 (2C); 103.5; 62.4; 47.4; 38.8; 38.7 (2C); 32.4 (2C); 31.9 (2C); 29.1; 28.6; 26.0.

UPLC: R_{T}: 1.62 (classic system)

MS (ESI⁺) m/z [M+H]⁺ Calcd for C₂₀H₂₇N₂O⁺ 311.21; Found 311.30.

### LYS19 Compound 6: LC340 (=Compound (015))

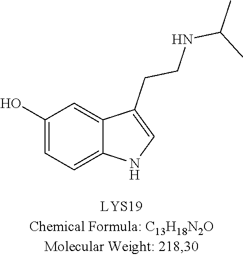 [SMILES: CC(C)NCCc1c[nH]c2ccc(O)cc12]

Yield: 70 mg, 32%. Isolated as a white amorphous solid, >95% pure by NMR and a single spot by TLC;

R_{f}: 0.29 in DCM/MeOH/Et₃N, 90/10/2. Strain green with CMA;

¹H NMR (CD₃OD, 400 MHZ): δ ppm=7.18 (1H, d, J=8.6 Hz); 7.04 (1H, s); 6.93 (1H, d, J=2.3 Hz); 6.68 (1H, dd, J=2.3 Hz, J=8.6 Hz); 3.05-2.91 (5H, m); 1.14 (6H, d, J=6.5 Hz).

¹³C NMR (CD₃OD, 101 MHZ): δ ppm=151.3; 133.2; 129.2; 124.5; 112.8; 112.6; 111.6; 103.3; 50.3; 47.7; 25.4; 21.4 (2C).

UPLC: R_{T}: 1.13 (classic system)

MS (ESI⁺) m/z [M+H]⁺ Calcd for C₁₃H₁₉N₂O⁺ 219.14; Found 219.21.

### LYS20 Compound 7: LC339 (=Compound (016))

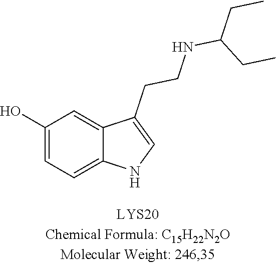 [SMILES: CCC(CC)NCCc1c[nH]c2ccc(O)cc12]

Yield: 57 mg, 23%. Isolated as a white amorphous solid, >95% pure by NMR and a single spot by TLC;

R_{f}: 0.63 in DCM/MeOH/Et₃N, 90/10/2. Strain green with CMA;

¹H NMR (CD₃OD, 400 MHZ): δ ppm=7.18 (1H, d, J=8.6 Hz); 7.07 (1H, s); 6.94 (1H, d, J=2.4 Hz); 6.69 (1H, dd, J=2.3 Hz, J=8.7 Hz); 3.06 (2H, m); 2.99 (2H, m); 2.68 (1H, quint., J=6.2 Hz); 1.56 (4H, m); 0.87 (6H, t, J=7.5 Hz).

¹³C NMR (CD₃OD, 101 MHZ): δ ppm=151.4; 133.2; 129.1; 124.7; 112.9; 112.7; 111.1; 103.3; 61.6; 47.3; 24.9 (3C); 9.8 (2C).

UPLC: R_{T}: 1.40 (classic system)

MS (ESI⁺) m/z [M+H]⁺ Calcd for C₁₅H₂₃N₂O⁺ 247.17; Found 247.20.

### LC347 (=Compound (017))

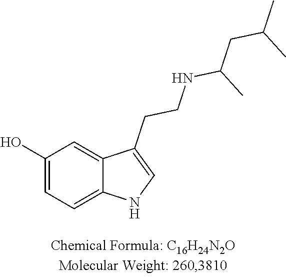 [SMILES: CC(C)CC(C)NCCc1c[nH]c2ccc(O)cc12]

Yield: 115 mg, 44%. Isolated as a white amorphous solid, >95% pure by NMR and a single spot by TLC;

R_{f}: 0.8 in DCM/MeOH/Et₃N, 90/10/2. Strain green with CMA;

¹H NMR (CD₃OD, 400 MHZ): δ ppm=7.17 (1H, dd, J=0.5 Hz, J=8.6 Hz); 7.05 (1H, s); 6.93 (1H, dd, J=0.5 Hz, J=2.3 Hz); 6.68 (1H, dd, J=2.3 Hz, J=8.6 Hz); 3.08-2.85 (5H, m); 1.53 (1H, m); 1.36 (1H, m); 1.24 (1H, m); 1.12 (3H, m); 0.88 (6H, dd, J=3.6 Hz, J=6.6 Hz).

¹³C NMR (CD₃OD, 101 MHz): δ ppm=151.3; 133.2; 129.1; 124.6; 112.9; 112.6; 111.4; 103.3; 52.9; 47.3; 45.8; 25.9; 25.3; 23.4; 22.3; 18.8.

UPLC: R_{T}: 1.58 (classic system)

MS (ESI⁺) m/z [M+H]⁺ Calcd for C₁₅H₂₃N₂O⁺ 261.19; Found 261.27.

### LYS23 Compound 8: LC349 (=Compound (019))

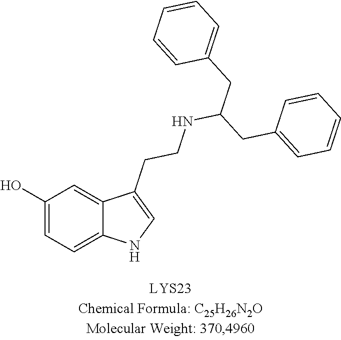 [SMILES: Oc1ccc2[nH]cc(CCNC(Cc3ccccc3)Cc3ccccc3)c2c1]

Yield: 131 mg, 35%. Isolated as a white amorphous solid, >95% pure by NMR and a single spot by TLC;

R_{f}: 0.33 in DCM/MeOH, 90/10. Strain green with CMA;

¹H NMR (CD₃OD, 400 MHZ): δ ppm=7.21-7.10 (7H, m); 7.01 (4H, m); 6.84 (1H, d, J=2.4 Hz); 7.07 (1H, s); 6.94 (1H, d, J=2.4 Hz); 6.67 (1H, dd, J=2.4 Hz, J=8.6 Hz); 6.62 (1H, s); 3.04 (1H, p, J=6.9 Hz); 2.92 (2H, t, J=6.6 Hz); 2.78 (1H, t, J=6.6 Hz); 2.72 (2H, dd, J=6.9 Hz, J=13.8 Hz); 2.61 (2H, dd, J=6.6 Hz, J=13.8 Hz).

¹³C NMR (CD₃OD, 101 MHz): δ ppm=151.1; 139.7 (2C); 133.2; 130.5 (4C); 129.6 (4C); 128.9; 127.5 (2C); 124.5; 112.8; 112.5; 111.3; 103.4; 62.5; 47.6; 40.8 (2C); 25.5.

UPLC: R_{T}: 1.91 (classic system)

MS (ESI⁺) m/z [M+H]⁺ Calcd for C₂₅H₂₇N₂O⁺ 371.20; Found 371.27.

### LC350 (=Compound (020))

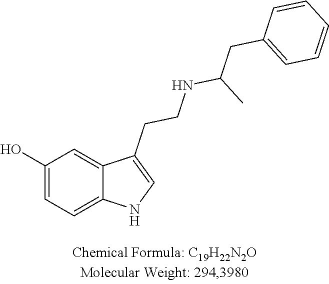 [SMILES: CC(Cc1ccccc1)NCCc1c[nH]c2ccc(O)cc12]

Yield: 187 mg, 63%. Isolated as a white amorphous solid, >95% pure by NMR and a single spot by TLC;

R_{f}: 0.6 in DCM/MeOH, 90/10. Strain green with CMA;

¹H NMR (CD₃OD, 400 MHZ): δ ppm=7.18-7.12 (4H, m); 7.01 (2H, dd, J=2.0 Hz, J=7.8 Hz); 6.90 (1H, d, J=0.5 Hz, J=2.3 Hz); 6.82 (1H, s); 6.68 (1H, dd, J=2.3 Hz, J=8.6 Hz); 3.0.3-2.81 (5H, m); 2.69 (1H, J=7.1 Hz, J=12.9 Hz); 2.59 (1H, J=7.1 Hz, J=12.9 Hz); 1.06 (3H, d, J=6.5 Hz).

¹³C NMR (CD₃OD, 101 MHZ): δ ppm=151.1; 139.7; 133.2; 130.1 (2C); 129.5 (2C); 129.1; 127.4; 124.4; 112.8; 112.5; 111.8; 103.4; 55.8; 47.6; 43.5; 25.7; 19.4.

UPLC: R_{T}: 1.62 (classic system)

MS (ESI⁺) m/z [M+H]⁺ Calcd for C₁₅H₂₃N₂O⁺ 295.17; Found 295.28.

### LC354 (=Compound (018))

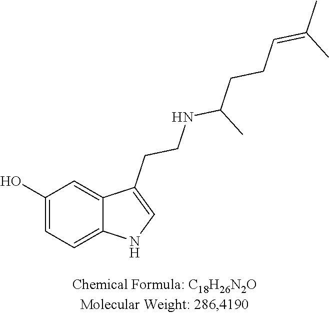 [SMILES: CC(C)=CCCC(C)NCCc1c[nH]c2ccc(O)cc12]

Yield: 273 mg, 95%. Isolated as a white amorphous solid, >95% pure by NMR and a single spot by TLC;

R_{f}: 0.12 in DCM/MeOH/Et₃N, 95/5/2. Strain green with CMA;

¹H NMR (CD₃OD, 400 MHZ): δ ppm=7.19 (1H, d, J=0.4 Hz, J=8.6 Hz); 7.08 (1H, s); 6.94 (1H, d, J=0.5 Hz, J=2.2 Hz); 6.70 (1H, dd, J=2.3 Hz, J=8.7 Hz); 5.06 (1H, m); 3.14-2.91 (5H, m); 1.98 (2H, m); 1.66 (3H, J=0.7 Hz); 1.65-1.60 (1H, m); 1.56 (3H, s); 1.46-1.36 (1H, m); 1.18 (3H, d, J=6.5 Hz).

¹³C NMR (CD₃OD, 101 MHZ): δ ppm=151.4; 133.5; 133.2; 129.1; 124.7; 124.2; 112.9; 112.7; 110.9; 103.3; 54.6; 47.1; 35.8; 25.8; 25.1; 24.9; 18.0; 17.7.

UPLC: R_{T}: 1.71 (classic system)

MS (ESI⁺) m/z [M+H]⁺ Calcd for C₁₅H₂₃N₂O⁺ 287.20; Found 287.30.

### LYS25 Compound X: LC357 (=Compound (021))

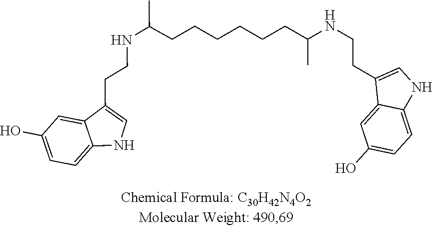 [SMILES: CC(CCCCCCC(C)NCCc1c[nH]c2ccc(O)cc12)NCCc1c[nH]c2ccc(O)cc12]

Yield: 36 mg, 16%. Isolated as a white amorphous solid, >95% pure by NMR and a single spot by TLC;

R_{f}: 0.32 in DCM/MeOH, 90/10+2% Et₃N. Strain green with CMA;

¹H NMR (CD₃OD, 500 MHZ): δ ppm=8.54 (2H, s, (FA)); 7.20 (2H, d, J=8.6 Hz); 7.11 (2H, s); 6.92 (2H, d, J=2.3 Hz); 6.71 (2H, dd, J=2.3 Hz, J=8.6 Hz); 3.29-3.16 (6H, m); 3.07 (4H, m); 1.71 (2H, m); 1.48 (2H, m); 1.41-1.22 (14H, m).

¹³C NMR (CD₃OD, 126 MHZ): δ ppm=170.1 (FA) (2C); 151.6 (2C); 133.2 (2C); 128.8 (2C); 124.9 (2C); 113.1 (2C); 112.9 (2C); 109.4 (2C); 103.1 (2C); 55.5 (2C); 46.1 (2C); 34.1 (2C); 30.2 (2C); 26.4 (2C); 23.7 (2C); 16.6 (2C).

UPLC: R_{T}: 1.48 (classic system)

MS (ESI⁺) m/z [M+H]⁺ Calcd for C₃₀H₄₂N₄O₂⁺ 491.33; Found 491.49.

3-2-2—Procedure from Aldehyde (Singly Substituted Analogs) [Compound 8 to x]:

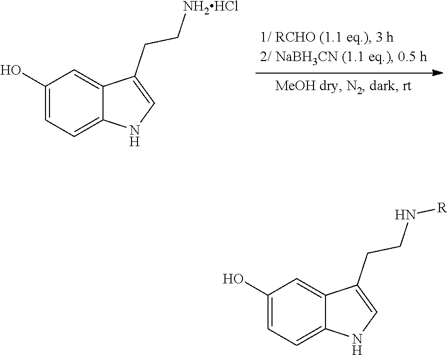

In the dark and under inert atmosphere, serotonin hydrochloride (213 mg, 1 mmol) was dissolved in MeOH dry (10 mL) and the corresponding aldehyde (1.1 equiv.) was added. The mixture was stirred for 3 h, and after that time NaBH₃CN (1.1 equiv.) was added. The reaction mixture was stirred further at RT for additional 30 minutes. Next, the solvent was evaporated under reduced pressure. The crude was taken in a mixture of Et₂O/water, 1/1 (10/10 mL), the resulting solution was alkalinized with NaOH [2M] until to obtain pH=10, then extracted with Et₂O (2×20 mL), then with DCM (1×20 mL). The organic phases were dried over MgSO₄ and concentrated. The crude was purified by flash chromatography using DCM/MeOH/Et₃N (100/0/2 to 80/20/2) as eluent.

### LYS29 (LC358) (=Compound (024))

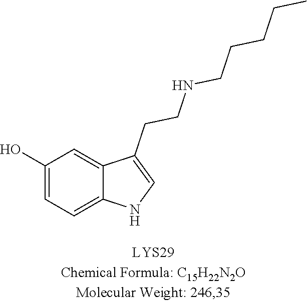 [SMILES: CCCCCNCCc1c[nH]c2ccc(O)cc12]

Yield: 88 mg, 36%. Isolated as a white amorphous solid, >95% pure by NMR and a single spot by TLC;

R_{f}: 0.31 in DCM/MeOH/Et₃N, 98/2/2. Strain green with CMA;

¹H NMR (CD₃OD, 500 MHZ): δ ppm=8.58 (1H, s, (FA)); 7.19 (1H, d, J=8.7 Hz); 7.09 (1H, s); 6.95 (1H, d, J=2.2 Hz); 6.71 (1H, dd, J=2.2 Hz, J=8.7 Hz); 3.21 (2H, t, J=7.5 Hz); 3.07 (2H, t, J=7.5 Hz); 2.94 (2H, t, J=7.9 Hz); 1.65 (2H, quint, J=7.8 Hz); 1.33 (4H, m); 0.92 (3H, t, J=6.7 Hz).

¹³C NMR (CD₃OD, 126 MHZ): δ ppm=170.3; 151.5; 133.1; 128.9; 124.9; 113.0; 112.8; 109.4; 103.1; 49.0; 48.8; 29.7; 26.9; 23.4; 23.2; 14.1.

UPLC: R_{T}: 1.45 (classic system)

MS (ESI⁺) m/z [M+H]⁺ Calcd for C₁₅H₂₂N₂O⁺ 247.17; Found 247.27.

### LYS30 Compound 9: LC343, LC345, LC346 (=Compound (025))

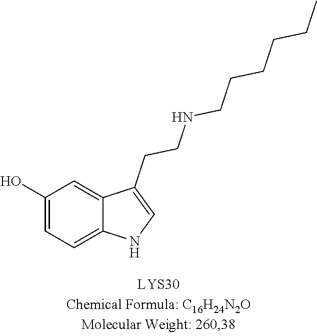 [SMILES: CCCCCCNCCc1c[nH]c2ccc(O)cc12]

Yield: 122 mg, 47%. Isolated as a white amorphous solid, >95% pure by NMR and a single spot by TLC;

R_{f}: 0.16 in DCM/MeOH/Et₃N, 98/2/2. Strain green with CMA;

¹H NMR (CD₃OD, 400 MHZ): δ ppm=7.16 (1H, d, J=8.7 Hz); 7.01 (1H, s); 6.92 (1H, d, J=2.1 Hz); 6.67 (1H, dd, J=2.1 Hz, J=8.7 Hz); 2.91 (4H, s); 2.62 (2H, m); 1.48 (2H, m); 1.26 (6H, m); 0.88 (3H, t, J=6.5 Hz).

¹³C NMR (CD₃OD, 101 MHZ): δ ppm=151.2; 133.2; 129.3; 124.3; 112.8; 112.5; 112.2; 103.4; 50.6; 50.3; 32.8; 30.0; 27.9; 25.8; 23.6; 14.3.

UPLC: R_{T}: 1.68 (classic system)

MS (ESI⁺) m/z [M+H]⁺ Calcd for C₁₆H₂₅N₂O₁₀⁺ 261.19; Found 261.19.

### LYS26 Compound 10: (Exp: LT266a) (=Compound (022))

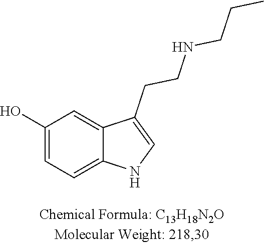 [SMILES: CCCNCCc1c[nH]c2ccc(O)cc12]

Yield: 10 mg, 4.5%. Isolated as a white amorphous solid, >95% pure by NMR and a single spot by TLC;

R_{f}: 0.3 in DCM/MeOH/Et₃N, 80/20/2. Strain green with CMA;

¹H NMR (CD₃OD, 400 MHZ): δ ppm=8.57 (1H, s, (FA)); 7.21 (1H, d, J=8.6 Hz); 7.13 (1H, s); 6.95 (1H, d, J=2.3 Hz); 6.72 (1H, dd, J=8.6 Hz, J=2.3 Hz); 3.28 (2H, t, J=7.6 Hz); 3.10 (2H, t, J=7.6 Hz); 2.99 (2H, d, J=8.6 Hz). 1.72 (2H, d, J=7.5 Hz); 1.02 (3H, t, J=7.4 Hz).

¹³C NMR (CD₃OD, 126 MHZ): δ ppm=168.7 (FA); 150.2; 131.8; 127.5; 123.5; 111.6; 111.5; 108.0; 101.7; 49.0; 22.0; 19.2; 9.8.

UPLC: R_{T}: 0.9 to 1.12 (classic system)

MS (ESI⁺) m/z [M+H]⁺ Calcd for C₁₃H₁₉N₂O⁺ 219.14; Found 219.26.

### LYS28 Compound 11: (Exp: LT265B) (=Compound (023))

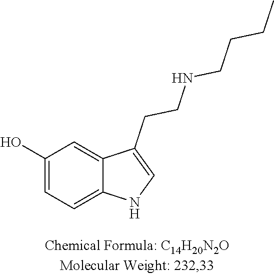 [SMILES: CCCCNCCc1c[nH]c2ccc(O)cc12]

Yield: 27.6 mg, 11.8%. Isolated as a white amorphous solid, >95% pure by NMR and a single spot by TLC; R_{f}: 0.5 in DCM/MeOH/Et₃N, 90/10/2. Strain green with CMA;

¹H NMR (CD₃OD, 500 MHZ): δ ppm=8.58 (s, 1H (FA)); 7.21 (1H, dd, J=8.7 Hz, J=2.7 Hz); 7.12 (1H, s); 6.96 (1H, d, J=2.4 Hz); 6.72 (1H, dd, J=8.6 Hz, J=2.0 Hz); 3.27 (2H, t, J=7.6 Hz); 3.10 (2H, t, J=7.6 Hz); 3.00 (2H, t, J=8.2 Hz); 1.67 (2H, p, J=7.9 Hz); 1.42 (2H, h, J=7.4 Hz); 0.98 (3H, t, J=7.4 Hz).

¹³C NMR (CD₃OD, 126 MHZ): δ ppm=168.9; 150.2; 131.8; 127.5; 123.5; 111.6; 111.5; 108.0; 101.7; 27.8; 22.1; 19.4; 12.5.

UPLC: R_{T}: 1.23 (classic system)

MS (ESI⁺) m/z [M+H]⁺ Calcd for C₁₄H₂₁N₂O⁺ 233.16; Found 233.20.

### LYS31 Compound 12: (Exp: LT265C) (=Compound (026))

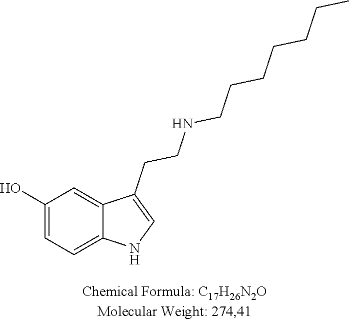 [SMILES: CCCCCCCNCCc1c[nH]c2ccc(O)cc12]

Yield: 71.8 mg, 26%. Isolated as a white amorphous solid, >95% pure by NMR and a single spot by TLC;

R_{f}: 0.6 in DCM/MeOH/Et₃N, 90/10/2. Strain green with CMA;

¹H NMR (CD₃OD, 500 MHZ): δ ppm=8.59 (1H, s, (FA)); 7.21 (1H, d, J=8.7 Hz); 7.12 (1H, s); 6.96 (1H, d, J=2.5 Hz); 6.72 (1H, dd, J=8.7 Hz, J=2.0 Hz); 3.26 (2H, dd, J=6.5 Hz, J=5.5 Hz); 3.09 (2H, t, J=7.6 Hz), 2.98 (2H, dd, J=6.6 Hz, J=5.3 Hz); 1.73-1.58 (2H, m); 1.44-1.25 (8H, m); 0.93 (3H, t, J=7.6 Hz).

¹³C NMR (CD₃OD, 126 MHZ): δ ppm=169.0, 150.2, 131.8, 127.5, 123.5, 111.6, 111.5, 108.1, 101.7, 31.3, 28.5, 26.2, 25.9, 22.2, 22.1, 13.0 ppm.

UPLC: R_{T}: 1.61 (classic system)

MS (ESI⁺) m/z [M+H]⁺ Calcd for C₁₇H₂₇N₂O⁺ 275.20; Found 275.28.

### LYS32 Compound 13: (Exp: LT266B) (=Compound (063))

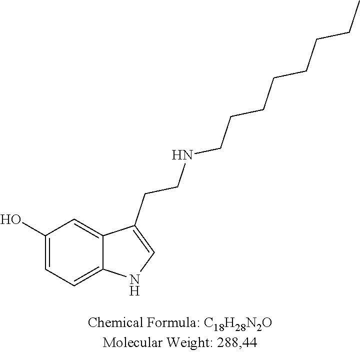 [SMILES: CCCCCCCCNCCc1c[nH]c2ccc(O)cc12]

Yield: 69 mg, 24%. Isolated as a white amorphous solid, >95% pure by NMR and a single spot by TLC;

R_{f}: 0.4 in DCM/MeOH/Et₃N, 90/10/2. Strain green with CMA;

¹H NMR (CD₃OD, 500 MHZ): δ ppm=8.60 (1H, d, J=2.3 Hz, (FA)); 7.21 (1H, d, J=8.6 Hz); 7.11 (1H, s); 6.97 (1H, d, J=2.4 Hz); 6.73 (1H, dd, J=8.6 Hz, J=2.3 Hz); 3.28-3.19 (2H, m); 3.08 (2H, t, J=7.6 Hz); 3.04-2.88 (2H, m); 1.75-1.60 (2H, m); 1.43-1.23 (10H, m); 0.92 (3H, t, J=6.7 Hz).

¹³C NMR (CD₃OD, 126 MHz): δ ppm=169.0; 150.2; 131.8; 127.5; 123.5; 111.6; 111.5; 108.1; 101.8; 31.5; 28.8 (2C); 26.2; 25.8; 22.3; 22.1; 13.0.

UPLC: R_{T}: 1.85 (classic system)

MS (ESI⁺) m/z [M+H]⁺ Calcd for C₁₈H₂₉N₂O⁺ 289.22; Found 289.22.

### LYS33 Compound 14: (Exp: LT262A) (=Compound (027))

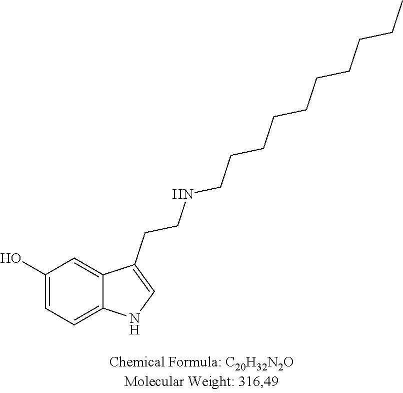 [SMILES: CCCCCCCCCCNCCc1c[nH]c2ccc(O)cc12]

Yield: 22 mg, 7%. Isolated as a white amorphous solid, >95% pure by NMR and a single spot by TLC; R_{f}: 0.7 in DCM/MeOH/Et₃N, 80/20/2. Strain green with CMA;

¹H NMR (CD₃OD, 500 MHz): δ ppm=8.59 (1H, s, (FA)); 7.21 (1H, d, J=8.6 Hz); 7.12 (1H, s); 6.96 (1H, d, J=2.3 Hz); 6.72 (1H, dd, J=8.6 Hz, J=2.3 Hz); 3.26 (2H, t, J=7.6 Hz); 3.09 (2H, t, J=7.6 Hz); 2.99 (2H, t, J=7.1 Hz); 1.67 (2H, p, J=7.4 Hz); 1.46-1.19 (14H, m); 0.92 (3H, t, J=6.8 Hz).

¹³C NMR (CD₃OD, 126 MHz): δ ppm=169.0; 150.2; 131.8; 127.5; 123.5; 111.6; 111.5; 108.1; 101.7; 31.6; 29.2; 29.1; 29.0; 28.8; 26.2; 25.9; 22.3; 22.1; 13.0.

UPLC: R_{T}: 2.03 (classic system)

MS (ESI⁺) m/z [M+H]⁺ Calcd for C₂₀H₃₃N₂O⁺ 317.25; Found 317.03.

### LYS34 Compound 15: (Exp: LT266C) (=Compound (064))

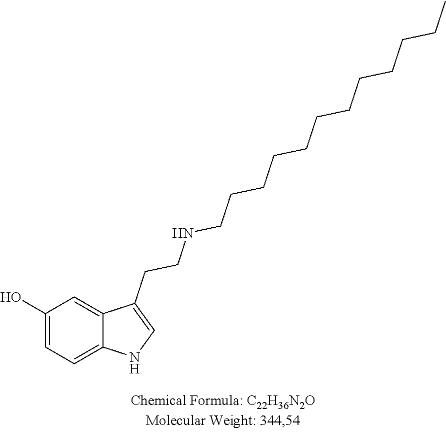 [SMILES: CCCCCCCCCCCCNCCc1c[nH]c2ccc(O)cc12]

Yield: 77 mg, 22%. Isolated as a white amorphous solid, >95% pure by NMR and a single spot by TLC;

R_{f}: 0.4 in DCM/MeOH/Et₃N, 90/10/2. Strain green with CMA. ¹H NMR (CD₃OD, 500 MHZ): δ ppm=8.60 (1H, s, (FA)); 7.21 (1H, d, J=8.7 Hz); 7.12 (1H, s); 6.96 (1H, d, J=2.2 Hz); 6.72 (1H, dd, J=8.7 Hz, J=2.3 Hz); 3.25 (2H, t, J=7.6 Hz); 3.09 (2H, t, J=7.6 Hz); 2.97 (2H, t, J=8.0 Hz); 1.67 (2H, p, J=7.5 Hz); 1.47-1.21 (18H, m); 0.92 (3H, t, J=6.9 Hz). ¹³C NMR (CD₃OD, 126 MHz): δ ppm=169.0; 150.2; 131.8; 127.5; 123.5; 111.6; 111.5; 108.1; 101.8; 31.7; 29.3 (2C); 29.2; 29.1; 29.1; 28.8; 26.2; 25.9; 22.3; 22.2; 13.1.

UPLC: R_{T}: 2.32 (classic system). MS (ESI⁺) m/z [M+H]⁺ Calcd for C₂₂H₃₇N₂O⁺ 345.28; Found 345.16.

### LYS35 Compound 16: LC355 (=Compound (028))

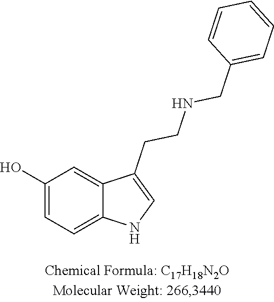 [SMILES: Oc1ccc2[nH]cc(CCNCc3ccccc3)c2c1]

Yield: 93 mg, 35%. Isolated as a white amorphous solid, >95% pure by NMR and a single spot by TLC;

R_{f}: 0.46 in DCM/MeOH/Et₃N, 98/2/2. ¹H NMR (CD₃OD, 400 MHZ): δ ppm=7.31-7.21 (5H, m); 7.16 (1H, d, J=8.6 Hz); 6.98 (1H, s); 6.90 (1H, d, J=2.4 Hz); 6.66 (1H, dd, J=8.6 Hz, J=2.4 Hz); 3.78 (2H, s); 2.91 (4H, s). ¹³C NMR (CD₃OD, 101 MHZ): δ ppm=151.2; 139.9; 133.2; 129.6 (2C); 129.5 (2C); 129.3; 128.3; 124.3; 112.7; 112.5; 112.4; 103.5; 54.2; 50.1; 25.9. UPLC: R_{T}: 1.48 (classic system). MS (ESI⁺) m/z [M+H]⁺ Calcd for C₁₇H₁₉N₂O⁺ 267.14; Found 267.21.

### LYS36 Compound 17: LC353B (=Compound (029))

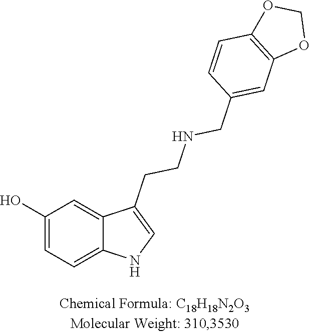 [SMILES: Oc1ccc2[nH]cc(CCNCc3ccc4c(c3)OCO4)c2c1]

Yield: 154 mg, 49%. Isolated as a white amorphous solid, >95% pure by NMR and a single spot by TLC;

R_{f}: 0.10 in DCM/MeOH, 95/5. ¹H NMR (CD₃OD, 400 MHZ): δ ppm=7.16 (1H, d, J=8.8 Hz); 6.98 (1H, s); 6.89 (1H, d, J=2.4 Hz); 6.98 (1H, s); 6.76 (1H, brs); 6.71 (2H, brs); 6.66 (1H, dd, J=8.7 Hz, J=2.4 Hz); 5.90 (2H, s); 3.68 (2H, s); 2.89 (4H, brt, J=3.9 Hz). ¹³C NMR (CD₃OD, 101 MHZ): δ ppm=151.1; 149.2; 148.3; 133.8; 133.2; 129.3; 124.3; 122.9; 112.7; 112.4 (2C); 109.8; 108.9; 103.5; 102.3; 53.9; 49.8; 25.9.

UPLC: R_{T}: 1.49 (classic system). MS (ESI⁺) m/z [M+H]⁺ Calcd for C₁₈H₁₉N₂O⁺ 311.13; Found 311.23.

### LC353A (=Compound (043))

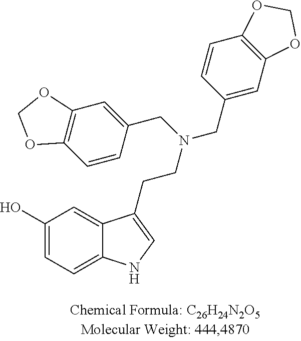 [SMILES: Oc1ccc2[nH]cc(CCN(Cc3ccc4c(c3)OCO4)Cc3ccc4c(c3)OCO4)c2c1]

Yield: 82 mg, 18%. Isolated as a white amorphous solid, >95% pure by NMR and a single spot by TLC;

R_{f}: 0.49 in DCM/MeOH, 95/5.

¹H NMR (CD₃OD, 400 MHZ): δ ppm=7.12 (1H, d, J=8.5 Hz); 6.88 (1H, s); 6.82 (2H, d, J=1.6 Hz); 6.75 (3H, m); 6.70 (2H, m); 6.62 (1H, dd, J=8.6 Hz, J=2.3 Hz); 5.89 (4H, s); 3.53 (4H, s); 2.84 (2H, m); 2.70 (2H, s).

¹³C NMR (CD₃OD, 101 MHZ): δ ppm=150.8; 149.1 (2C); 148.0 (2C); 134.8 (2C); 133.1; 129.5; 123.9; 123.1 (2C); 113.7; 112.5; 112.1; 110.1 (2C); 108.7 (2C); 103.7; 102.1 (2C); 58.9 (2C); 54.9; 24.0.

UPLC: R_{T}: 1.79 (classic system)

MS (ESI⁺) m/z [M+H]⁺ Calcd for C₂₆H₂₅N₂O₅⁺ 445.17; Found 445.23.

### LYS37 Compound 18: (Exp: LT262B) (=Compound (058))

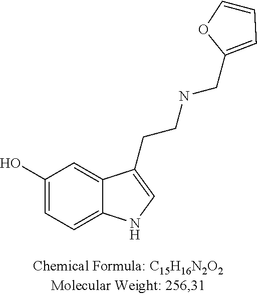 [SMILES: Oc1ccc2[nH]cc(CCNCc3ccco3)c2c1]

Yield: 31 mg, 12%. Isolated as a white amorphous solid, >95% pure by NMR and a single spot by TLC;

R_{f}: 0.5 in DCM/MeOH/Et₃N, 90/10/2. Strain green with CMA. ¹H NMR (CD₃OD, 500 MHZ): δ ppm=8.53 (1H, s); 7.63 (1H, d, J=1.8 Hz); 7.21 (1H, d, J=8.7 Hz); 7.11 (1H, s); 6.93 (1H, d, J=2.3 Hz); 6.72 (1H, dd, J=8.7 Hz, J=2.3 Hz); 6.61 (1H, d, J=3.3 Hz); 6.50 (1H, dd, J=3.3, 1.8 Hz); 4.27 (2H, s); 3.27 (2H, t, J=7.7 Hz); 3.09 (2H, t, J=7.6 Hz). ¹³C NMR (CD₃OD, 126 MHZ): δ ppm=168.4; 150.2; 145.4; 144.3; 131.8; 127.4; 123.5; 112.0; 111.6; 111.5; 110.7; 108.0; 101.8; 47.0; 42.7; 22.0. UPLC: R_{T}: 1.22 (classic system). MS (ESI⁺) m/z [M+H]⁺ Calcd for C₁₅H₁₇N₂O⁺ 257.12; Found 257.14.

### LYS38 Compound 19: (Exp: LT267C) (=Compound (059))

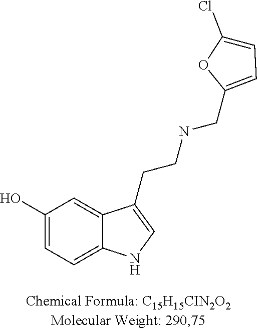 [SMILES: Oc1ccc2[nH]cc(CCNCc3ccc(Cl)o3)c2c1]

Yield: 37.3 mg, 13%. Isolated as a white amorphous solid, >95% pure by NMR and a single spot by TLC;

R_{f}: 0.4 in DCM/MeOH/Et₃N, 90/10/2. Strain green with CMA. ¹H NMR (CD₃OD, 400 MHZ): δ ppm=8.41 (1H, s); 7.09 (1H, d, J=8.6 Hz); 6.98 (1H, s); 6.81 (1H, d, J=2.2 Hz); 6.60 (1H, dd, J=8.7 Hz, J=2.3 Hz); 6.48 (1H, d, J=3.4 Hz); 6.22 (1H, d, J=3.3 Hz); 4.06 (2H, s), 3.12 (2H, t, J=7.6 Hz); 2.95 (2H, t, J=7.5 Hz). ¹³C NMR (CD₃OD, 101 MHZ): δ ppm=150.1; 146.4; 137.4; 131.8; 127.5; 123.5; 114.0; 111.6; 111.4; 108.3; 107.4; 101.8; 47.3; 43.0; 22.3. UPLC: R_{T}: 1.43 (classic system). MS (ESI⁺) m/z [M+H]⁺ Calcd for C₁₅H₁₆N₂O⁺ 291.08; Found 291.10.

### 3-3—N-cycloalkylation

Strategy: O-Silylation then N-Cycloalkylation

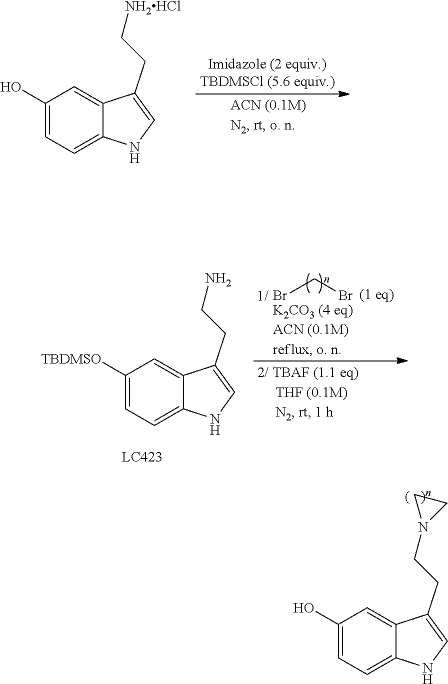

A mixture of serotonin hydrochloride (1.0 g, 4.7 mmol), tert-butyldimethylsilyl chloride (TBDMSCl, 3.97 g, 5.6 equiv.) and imidazole (640 mg, 2 equiv.) in acetonitrile (47 mL) was stirred at room temperature for overnight under N₂ and then was filtered. The solid residue was dissolved in 0.5 M NaOH aqueous solution (100 mL), and immediately extracted with CH₂Cl₂ (3×30 mL). The combined CH₂Cl₂ extracts were washed with deionized water (30 mL) and dried over Mg₂SO₄. Removing the solvent under reduced pressure afforded the desired compound LC423 as a greyish solid.

In the dark and under inert atmosphere, the product of the previous step LC423 (145 mg, 0.5 mmol, 1 equiv.) was dissolved in dry acetonitrile (4 mL). K₂CO₃ (276 mg, 4 equiv.) and dibromoalkyl (1 equiv.) were added to the solution. The mixture was stirred and heated at reflux overnight, cooled down to r.t. then filtered with acetonitrile and concentrated. The crude was purified by flash chromatography using cyclohexane/EtOAC (90/10 to 0/100) as eluent to give the O-silylated-N-cycloalkylated derivatives.

The product was then dissolved in THF (C=0.1M) and 1.1 equivalent of TBAF (1 M in THF) was added.

The mixture was stirred for 1 h until the O-deprotected product was predominant in UPLC analysis. The mixture was then concentrated and the crude was directly purified by preparative HPLC to give the desired product after lyophilization.

### LC423 (5-((tert-Butyldimethylsilyl)oxy-1H-indol-3-yl) ethanamine

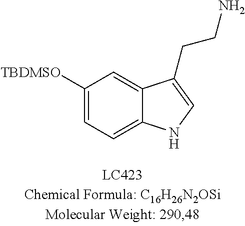 [SMILES: CC(C)(C)[Si](C)(C)Oc1ccc2[nH]cc(CCN)c2c1]

Yield: 1.29 g, 94%. Isolated as a light grey foam, >95% pure by NMR and a single spot by TLC; R_{f}: 0.15 in DCM/MeOH, 95/5+2% Et₃N. Strain green with CAM;

¹H NMR (CD₂Cl₂, 400 MHZ): δ ppm=8.17 (1H, brs); 7.20 (1H, dd, J=0.6 Hz, J=8.5 Hz); 7.01 (1H, d, J=2.1 Hz); 6.99 (1H, d, J=2.3 Hz); 6.73 (1H, dd, J=2.3 Hz, J=8.5 Hz); 2.95 (2H, t, J=7.0 Hz); 2.80 (2H, t, J=6.8 Hz); 1.31 (2H, brs); 1.01 (9H, s); 0.19 (6H, s).

¹³C NMR (CD₂Cl₂, 101 MHZ): δ ppm=148.9; 132.1; 128.3; 122.9; 115.9; 113.5; 111.3; 108.3; 42.5; 29.7; 25.6 (3C); 18.1; −3.8 (2C).

UPLC: R_{T}: 1.85 (classic system)

MS (ESI⁺) m/z [M+H]⁺ Calcd for C₁₆H₂₈N₂OSi⁺ 291.18; Found 291.18.

### LC434

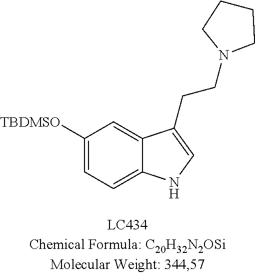 [SMILES: CC(C)(C)[Si](C)(C)Oc1ccc2[nH]cc(CCN3CCCC3)c2c1]

Yield: 70 mg, 41%. Isolated as a light grey foam, >95% pure by NMR and a single spot by TLC; R_{f}: 0.67 in DCM/MeOH, 95/5+2% Et₃N. Strain green with CAM;

¹H NMR (CD₃OD, 400 MHZ): δ ppm=7.19 (1H, dd, J=0.5 Hz, J=8.6 Hz); 7.04 (1H, s); 6.96 (1H, d, J=2.1 Hz); 6.67 (1H, dd, J=2.3 Hz, J=8.6 Hz); 2.92 (4H, m); 2.78 (4H, m); 1.88 (4H, m); 1.02 (9H, s); 0.19 (6H, s).

¹³C NMR (CD₃OD, 101 MHZ): δ ppm=149.7; 133.9; 129.1; 124.2; 116.5; 112.6 (2C); 108.7; 58.1; 55.1 (2C); 26.3 (3C); 25.3; 24.1 (2C); 19.1; −4.2 (2C).

UPLC: R_{T}: 2.05 (classic system)

MS (ESI⁺) m/z [M+H]⁺ Calcd for C₂₀H₃N₂OSi⁺ 345.57; Found 345.21.

### LC437

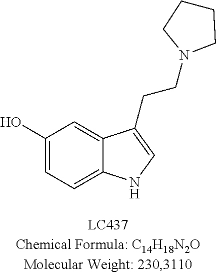 [SMILES: Oc1ccc2[nH]cc(CCN3CCCC3)c2c1]

Yield: 35 mg, 76%. Isolated as a light grey foam, >95% pure by NMR and a single spot by TLC; R_{f}: 0.41 in DCM/MeOH, 95/5+2% Et₃N. Strain green with CAM;

¹H NMR (CD₃OD, 500 MHz): δ ppm=8.51 (1H, s, FA); 7.19 (1H, d, J=8.5 Hz); 7.10 (1H, s); 6.96 (1H, brs); 6.70 (1H, brd, J=8.4 Hz); 3.37 (2H, d, J=7.5 Hz); 3.29 (4H, m); 3.08 (2H, t, J=7.8 Hz); 2.01 (4H, brs).

¹³C NMR (CD₃OD, 101 MHz): δ ppm=169.8 (FA); 151.6; 133.1; 128.8; 125.0; 113.1; 112.9; 109.2; 103.2; 56.5; 55.1 (2C); 23.9 (2C); 23.2.

UPLC: R_{T}: 0.61 (classic system)

MS (ESI⁺) m/z [M+H]⁺ Calcd for C₁₄H₁₉N₂O⁺ 231.14; Found 231.09.

### LC432F2-LC432 (=Compound (045))

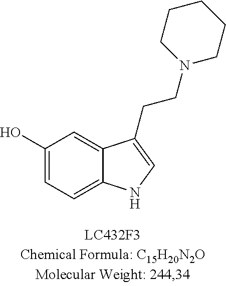 [SMILES: Oc1ccc2[nH]cc(CCN3CCCCC3)c2c1]

Comment: LC432F2 is obtained directly deprotected after the alkylation reaction.

Yield: 65 mg, 53%. Isolated as a light grey foam, >95% pure by NMR and a single spot by TLC; R_{f}: 0.59 in DCM/MeOH, 95/5+2% Et₃N. Strain green with CAM;

¹H NMR (CD₃OD, 400 MHZ): δ ppm=8.56 (1H, brs, FA); 7.18 (1H, d, J=8.6 Hz); 7.09 (1H, s); 6.93 (1H, d, J=2.1 Hz); 6.70 (1H, dd, J=2.4 Hz, J=8.5 Hz); 3.33-3.15 (6H, m); 3.12 (2H, m); 1.85 (4H, m); 1.67 (2H, brs).

¹³C NMR (CD₃OD, 101 MHz): δ ppm=170.2 (FA); 151.6; 133.1; 128.8; 124.7; 113.0; 112.8; 109.5; 103.1; 58.6; 54.3 (2C); 24.4 (2C); 22.9; 22.6.

UPLC: R_{T}: 1.15 (classic system)

MS (ESI⁺) m/z [M+H]⁺ Calcd for C₁₅H₂₁N₂O⁺ 244.16; Found 245.17.

### 3-4—N-dimer

### Strategy

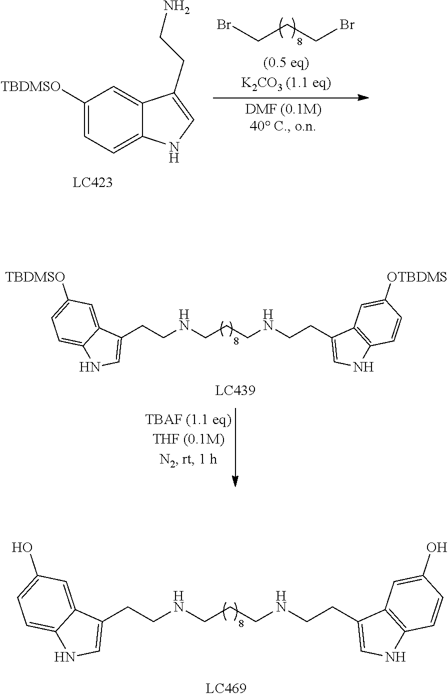

In the dark and under inert atmosphere, LC423 (145 mg, 0.5 mmol) was dissolved in dry DMF (1 mL). K₂CO₃ (76 mg, 1.1 equiv.) and 1,10-dibromodecane (75 mg, 0.5 equiv.) were added to the solution. The mixture was stirred at 40° C. overnight, cooled down to r.t. then concentrated to dryness. The crude was purified by flash chromatography using DCM/MeOH (100/0 to 90/10)+2% Et₃N as eluent, then by preparative HPLC to give the desired product LC439 after lyophilization.

LC439 was then dissolved in THF (C=0.1M) and 1.1 equivalent of TBAF (1 M in THF) was added. The mixture was stirred for 1 h until the O-deprotected product was predominant in UPLC analysis. The mixture was then concentrated and the crude was directly purified by preparative HPLC to give the desired product LC469 after lyophilization.

### LC439

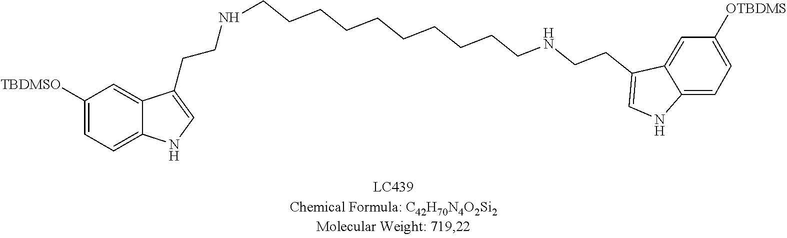 [SMILES: CC(C)(C)[Si](C)(C)Oc1ccc2[nH]cc(CCNCCCCCCCCCCNCCc3c[nH]c4ccc(O[Si](C)(C)C(C)(C)C)cc34)c2c1]

Yield: 34 mg, 9.5%. Isolated as a light grey foam, >95% pure by NMR and a single spot by TLC; R_{f}: 0.50 in DCM/MeOH, 90/10+2% Et₃N. Strain green with CAM;

¹H NMR (CD₃OD, 500 MHZ): δ ppm=8.59 (2H, brs, FA); 7.25 (2H, d, J=8.7 Hz); 7.16 (2H, s); 7.00 (2H, d, J=2.0 Hz); 6.72 (2H, dd, J=2.3 Hz, J=8.7 Hz); 3.25 (4H, t, J=7.5 Hz); 3.10 (4H, t, J=7.5 Hz); 2.98 (4H, m); 1.67 (4H, m); 1.34 (12H, m); 1.03 (18H, s); 0.21 (12H, s).

¹³C NMR (CD₃OD, 126 MHZ): δ ppm=168.9 (FA); 148.7 (2C); 132.6 (2C); 127.4 (2C); 123.7 (2C); 115.5 (2C); 111.5 (2C); 108.5 (2C); 107.1 (2C); 47.8 (2C); 47.5 (2C); 28.9 (2C); 28.8 (2C); 26.2 (2C); 25.9 (2C); 24.9 (6C); 22.1 (2C); 17; 7 (2C); −5.6 (4C).

UPLC: R_{T}: 2.43 (classic system)

MS (ESI⁺) m/z [M+H]⁺ Calcd for C₄₂H₇₁N₄O₂Si₂⁺ 719.50; Found 719.62.

### LC469 (=Compound (047))

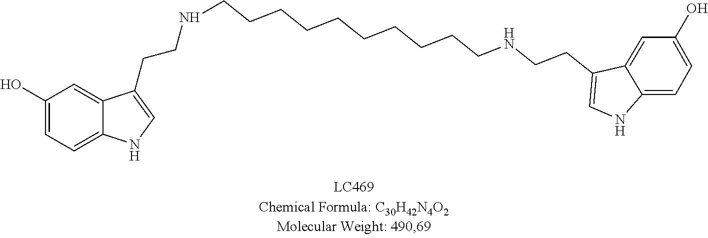 [SMILES: Oc1ccc2[nH]cc(CCNCCCCCCCCCCNCCc3c[nH]c4ccc(O)cc34)c2c1]

Yield: 18 mg, 78%. Isolated as a light grey foam, >95% pure by NMR;

¹H NMR (CD₃OD, 400 MHZ): δ ppm=8.53 (2H, brs, FA); 7.19 (2H, d, J=8.7 Hz); 7.10 (2H, s); 6.93 (2H, d, J=2.2 Hz); 6.70 (2H, dd, J=2.1 Hz, J=8.7 Hz); 3.26 (4H, t, J=7.5 Hz); 3.08 (4H, t, J=7.5 Hz); 2.99 (4H, m); 1.66 (4H, m); 1.34 (12H, m).

¹³C NMR (CD₃OD, 101 MHZ): δ ppm=170.1 (FA); 1516 (2C); 133.2 (2C); 128.9 (2C); 124.9 (2C); 113.0 (2C); 112.8 (2C); 109.4 (2C); 103.1 (2C); 49.1 (2C); 48.9 (2C); 30.4 (2C); 30.2 (2C); 27.6 (2C); 27.2 (2C); 23.5 (2C).

UPLC: R_{T}: 1.49 (classic system)

MS (ESI⁺) m/z [M+H]⁺ Calcd for C₄₂H₇₁N₄O₂Si₂⁺ 491.33; Found 491.56.

3-5—pyrrole C-alkylation

### 3-5-1—Strategy 1: N-et O-Acetyl

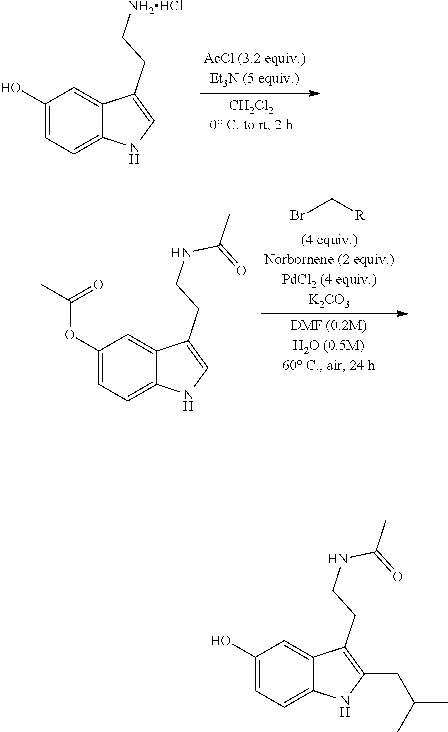

To a cold (0° C.) solution of serotonin hydrochloride (2.5 g, 11.5 mmol) and triethylamine (8.25 mL, 5 equiv.) in CH₂Cl₂ (80 mL) was added slowly acetyl chloride (2.67 mL, 3.2 equiv.) over 10 min. After the addition was complete, the reaction mixture was allowed to warm at room temperature and stirred for 2 hours. The reaction was diluted with CH₂Cl₂ (20 mL) and washed with water (2×20 mL) and brine (30 mL), dried over MgSO₄ and the solvent was evaporated to afford the compound LC377 as a pale yellow foam.

To a mixture of diacetylated serotonin derivative LC377 (130 mg, 0.50 mmol), norbornene (94 mg, 2.00 equiv), K₂CO₃ (276 mg, 4.00 equiv), and PdCl₂ (8.9 mg, 10 mol %) was added 2.5 mL of DMF (containing 0.5 M H₂O) and 1-bromo-2-methylpropane (218 μL, 4.00 equiv). The resulting suspension was stirred at 60° C. for 24 h. After being cooled to room temperature, the reaction mixture was diluted with Et₂O (30 mL) and washed with water (50 mL). The aqueous layer was then extracted with Et₂O (2×50 mL), and the combined organic extracts were dried over MgSO₄ and concentrated. The crude product was purified by flash column chromatography on silica gel to afford the 2-alkylserotonin product LC385. Comment: desacetylation of the phenol occurs during the reaction.

### LC377 (=Compound (048))

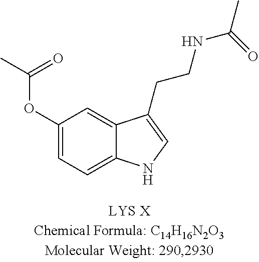 [SMILES: CC(=O)NCCc1c[nH]c2ccc(OC(C)=O)cc12]

Yield: 3.06 g, 100%. Isolated as a light yellow foam, >95% pure by NMR and a single spot by TLC; R_{f}: 0.5 in DCM/MeOH, 95/5. Strain green with CAM;

¹H NMR (DMSO-d6, 500 MHz): δ ppm=10.91 (1H, s); 7.92 (1H, brt, J=5.4 Hz); 7.35 (1H, d, J=8.6 Hz); 7.22 (2H, dd, J=2.4 Hz, J=7.9 Hz); 6.81 (1H, dd, J=2.2 Hz, J=8.6 Hz); 3.30 (2H, dd, J=6.0 Hz, J=7.0 Hz); 2.79 (2H, t, J=7.4 Hz); 2.26 (3H, s); 1.80 (3H, s).

¹³C NMR (DMSO-d₆, 126 MHZ): δ ppm=169.9; 169.1; 143.3; 134.0; 127.4; 124.2; 115.3; 112.2; 111.7; 110.4; 54.9; 25.1; 22.7; 20.9.

UPLC: R_{T}: 1.70 (classic system)

MS (ESI⁺) m/z [M+H]⁺ Calcd for C₁₄H₁₇N₂O₃⁺ 261.12; Found 261.21.

### LC385 (=Compound (049))

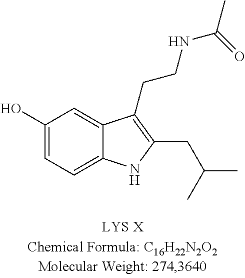 [SMILES: CC(=O)NCCc1c(CC(C)C)[nH]c2ccc(O)cc12]

Yield: 19 mg, 12%. Isolated as a light grey powder, >95% pure by NMR and a single spot by TLC; R_{f}: 0.64 in EtOAc/MeOH, 95/5. Strain green with CAM;

¹H NMR (CD₃OD, 500 MHZ): δ ppm=7.06 (1H, d, J=8.5 Hz); 6.87 (1H, d, J=2.3 Hz); 6.58 (1H, dd, J=2.3 Hz, J=8.5 Hz); 3.34 (2H, t, J=7.5 Hz); 2.81 (2H, t, J=7.5 Hz); 2.55 (2H, d, J=7.5 Hz); 1.99 (1H, quint, J=6.9 Hz); 1.91 (3H, s); 0.94 (6H, d, J=6.7 Hz).

¹³C NMR (CD₃OD, 126 MHZ): δ ppm=173.2; 150.9; 137.9; 132.1; 130.5; 111.8; 111.1; 108.6; 103.2; 41.5; 36.3; 30.4; 25.4; 22.9 (2C); 22.7.

UPLC: R_{T}: 1.87 (classic system)

MS (ESI⁺) m/z [M+H]⁺ Calcd for C₁₆H₂₃N₂O₂⁺ 275.17; Found 275.28.

### 3-5-2—Strategy2: NH-Boc et O-TBDMS

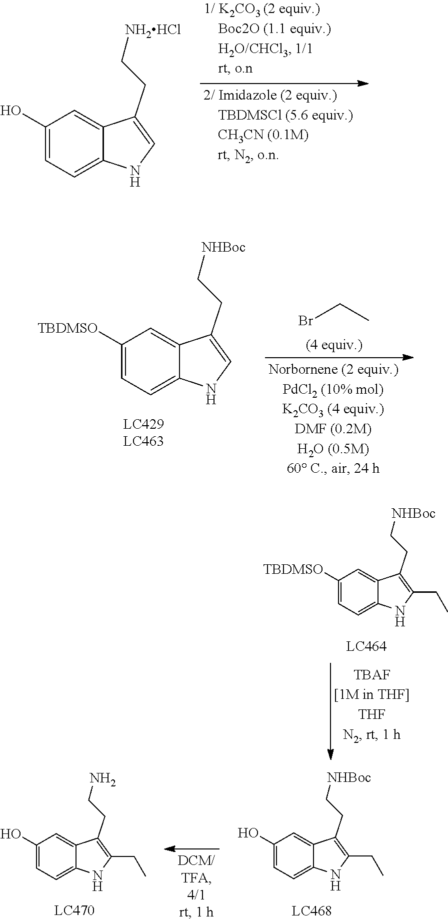

In the dark, serotonin hydrochloride (500 mg, 2.35 mmol, 1 equiv.) was dissolved in water (9 mL). K₂CO₃ (665 mg, 4.81 mmol, 2.1 equiv.) and Boc₂O (538 mg, 2.46 mmol, 1.05 equiv.) were added to the solution. The solution was stirred overnight, and then extracted with DCM. The organic phase was washed with aq. HCl 5% and brine, then dried on MgSO4 and concentrated. The crude was purified by flash chromatography using DCM/MeOH (100/0 to 90/10) as eluent to give the desired product (485 mg), which was engaged in the next step.

In the dark and under inert atmosphere, the product of the previous step (553 mg, 2 mmol), tert-butyldimethylsilyl chloride (TBDMSCl, 1.69 g, 5.6 equiv.) and imidazole (272 mg, 2 equiv.) in acetonitrile (20 mL) was stirred at room temperature for overnight under N₂ and then was filtered. The solid residue was dissolved in 0.5 M NaOH aqueous solution (50 mL), and immediately extracted with CH₂Cl₂ (3×20 mL). The combined CH₂Cl₂ extracts were washed with deionized water (30 mL) and dried over Mg₂SO₄. The crude was purified by flash chromatography using n-Hex/EtOAc (100/0 to 50/50) as eluent to give the desired product LC429 as a white solid.

To a mixture of LC429 serotonin derivative (117 mg, 0.30 mmol), norbornene (56 mg, 2.00 equiv), K₂CO₃ (166 mg, 4.00 equiv), and PdCl₂ (5.3 mg, 10 mol %) was added 2.1 mL of DMF (containing 0.5 M H₂O) and bromoethane (90 μL, 4.00 equiv). The resulting suspension was stirred at 60° C. for 24 h. After being cooled to room temperature, the reaction mixture was diluted with Et₂O (30 mL) and washed with water (50 mL). The aqueous layer was then extracted with Et₂O (2×50 mL), and the combined organic extracts were dried over MgSO₄ and concentrated. The crude product was purified by flash column chromatography on silica gel using n-Hex/EtOAc (100/0 to 50/50) as eluent to give LC464.

LC464 was dissolved in THF (C=0.1M) and 1.1 equivalent of TBAF (1 M in THF) was added. The mixture was stirred for 1 h until the O-deprotected product was predominant in UPLC analysis. The mixture was then concentrated and the crude was directly purified by preparative HPLC to give the desired product LC468 after lyophilization.

LC468 was dissolved in CH₂Cl₂/TFA, 4/1 (4 mL) and stirred for 2 h until the product was predominant in UPLC analysis. The mixture was then concentrated and the crude was directly purified by preparative HPLC to give the desired product LC470 after lyophilization.

### LC429

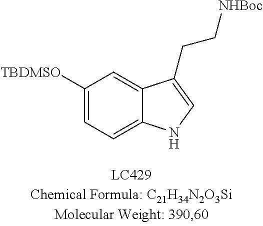 [SMILES: CC(C)(C)OC(=O)NCCc1c[nH]c2ccc(O[Si](C)(C)C(C)(C)C)cc12]

Yield: 691 mg, 88%. Isolated as a light grey powder, >95% pure by NMR and a single spot by TLC; R_{f}: 0.56 in n-Hex/EtOAc, 60/40. Strain green with ninhydrin;

¹H NMR (CD₃OD, 500 MHZ): δ ppm=7.17 (1H, d, J=8.6 Hz); 7.02 (1H, s); 6.98 (1H, brd, J=1.2 Hz); 6.66 (1H, dd, J=2.3 Hz, J=8.5 Hz); 3.29 (2H, t, J=7.3 Hz); 2.83 (2H, t, J=7.3 Hz); 1.43 (9H, s); 1.02 (9H, s); 0.19 (6H, s).

¹³C NMR (CD₃OD, 126 MHZ): δ ppm=158.5; 149.7; 133.9; 129.4; 124.3; 116.4; 112.9; 112.5; 108.9; 79.9; 42.4; 28.8 (3C); 26.8; 26.3 (3C); 19.1; −4.2 (2C).

UPLC: R_{T}: 3.04 (classic system)

MS (ESI⁺) m/z [M+Na]⁺ Calcd for C₂₁H₃₄N₂O₃SiNa⁺ 413.21; Found 413.29.

### LC464

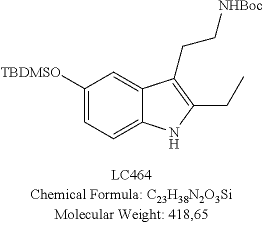 [SMILES: CCc1[nH]c2ccc(O[Si](C)(C)C(C)(C)C)cc2c1CCNC(=O)OC(C)(C)C]

Yield: 50 mg, 40%. Isolated as a light grey powder, >95% pure by NMR and a single spot by TLC; R_{f}: 0.65 in n-Hex/EtOAc, 70/30. Strain green with ninhydrin;

¹H NMR (CD₃OD, 500 MHZ): δ ppm=7.09 (1H, d, J=8.4 Hz); 6.90 (1H, brs); 6.57 (1H, dd, J=2.4 Hz, J=8.6 Hz); 3.20 (2H, t, J=7.0 Hz); 2.79 (2H, t, J=7.2 Hz); 2.73 (2H, q, J=7.7 Hz); 1.43 (9H, s); 1.28 (3H, t, J=7.5 Hz); 1.01 (9H, s); 0.18 (6H, s).

¹³C NMR (CD₃OD, 126 MHz): δ ppm=158.5; 149.6; 140.4; 132.9; 130.5; 115.2; 111.6; 108.5; 107.7; 79.8; 42.5; 28.8 (3C); 26.3 (3C); 25.7; 20.3; 19.1; 14.9; −4.2 (2C).

UPLC: R_{T}: 3.18 (classic system)

MS (ESI⁺) m/z [M+Na]⁺ Calcd for C₂₃H₃₈N₂O₃SiNa⁺ 441.23; Found 441.22.

### LC468 (=Compound (046))

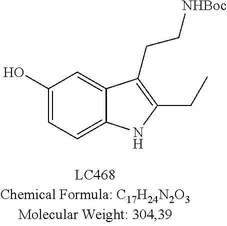 [SMILES: CCc1[nH]c2ccc(O)cc2c1CCNC(=O)OC(C)(C)C]

Yield: 20 mg, 56%. Isolated as a light grey powder, >95% pure by NMR and a single spot by TLC; R_{f}: 0.38 in n-Hex/EtOAc, 60/40. Strain green with ninhydrin;

¹H NMR (CD₃OD, 400 MHZ): δ ppm=7.06 (1H, dd, J=0.5 Hz, J=8.5 Hz); 6.85 (1H, brd, J=1.8 Hz); 6.57 (1H, dd, J=2.3 Hz, J=8.5 Hz); 3.20 (2H, t, J=7.0 Hz); 2.77 (2H, t, J=7.5 Hz); 2.72 (2H, q, J=7.7 Hz); 1.43 (9H, s); 1.27 (3H, t, J=7.5 Hz).

¹³C NMR (CD₃OD, 101 MHZ): δ ppm=158.5; 150.9; 140.2; 132.1; 130.6; 111.7; 111.0; 107.4; 103.3; 79.8; 42.3; 28.8 (3C); 25.8; 20.3; 14.9.

UPLC: R_{T}: 2.17 (classic system)

MS (ESI⁺) m/z [M+H]⁺ Calcd for C₁₇H₂₄N₂O₃Na⁺ 327.18; Found 327.46.

### LC470

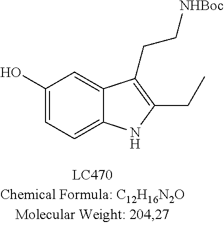 [SMILES: CCc1[nH]c2ccc(O)cc2c1CCNC(=O)OC(C)(C)C]

Yield: 10 mg, 77%. Isolated as a light grey powder, >95% pure by NMR;

¹H NMR (CD₃OD, 400 MHZ): δ ppm=7.11 (1H, dd, J=0.5 Hz, J=8.7 Hz); 6.84 (1H, d, J=2.3 Hz); 6.62 (1H, dd, J=2.4 Hz, J=8.7 Hz); 3.10 (2H, m); 2.99 (2H, m); 2.75 (2H, q, J=7.6 Hz); 1.29 (3H, t, J=7.6 Hz).

¹³C NMR (CD₃OD, 101 MHz): δ ppm=151.4; 140.9; 132.2; 130.0; 112.2; 111.5; 104.2; 102.8; 41.3; 23.5; 20.1; 14.8.

UPLC: R_{T}: 1.19 (classic system)

MS (ESI⁺) m/z [M+H]⁺ Calcd for C₁₂H₁₇N₂O⁺ 205.13; Found 188.13.

### 3-6—Serotonin/Pyrrole C-Trifluoromethylation

### Strategy

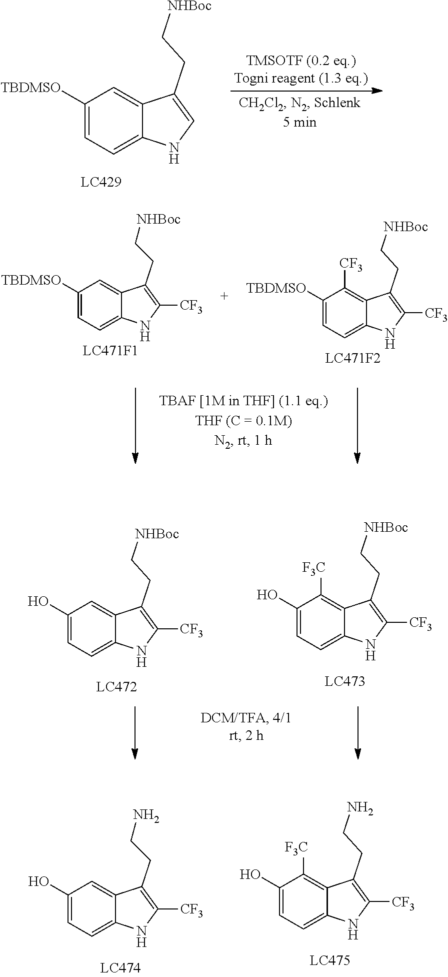

For the trifluoromethylation of indole, LC429 (195 mg, 0.5 mmol) and Togni reagent (342 mg, 1.3 equiv.) were added to a Schlenk tube. Under vacuum, DCM (5 mL) and TMSOTf (18 μL, 0.2 equiv.) were added and the mixture was stirred for 15 min. NaHCO₃ aq. sat. (15 mL) was added to quench the reaction mixture, then extracted with CH₂Cl₂ (3×20 mL). The combined CH₂Cl₂ extracts were dried over Mg₂SO₄. The crude was purified by flash chromatography using n-Hex/EtOAc (100/0 to 50/50) as eluent to give the desired product LC471F1 and a side product LC471F2 as white solids.

LC471F1 was dissolved in THF (C=0.1M) and 1.1 equivalent of TBAF (1 M in THF) was added. The mixture was stirred for 1 h until the O-deprotected product was predominant in UPLC analysis. NH₄Cl aq. sat. (15 mL) was added and extracted with EtOAc (3×20 mL). The combined organic phases were dried with MgSO₄. Removing the solvent under reduced pressure afforded the desired compound

LC472 which is used for the next step without purification.

LC471F2 was dissolved in THF (C=0.1M) and 1.1 equivalent of TBAF (1 M in THF) was added. The mixture was stirred for 1 h until the O-deprotected product was predominant in UPLC analysis. NH₄Cl aq. sat. (15 mL) was added and extracted with EtOAc (3×20 mL). The combined organic phases were dried with MgSO₄. Removing the solvent under reduced pressure afforded the desired compound

LC473 which is used for the next step without purification.

LC472 was dissolved in CH₂Cl₂/TFA, 4/1 (4 mL) and stirred for 2 h until the product was predominant in UPLC analysis. The mixture was then concentrated and the crude was directly purified by preparative HPLC to give the desired product LC474 after lyophilization.

LC473 was dissolved in CH₂Cl₂/TFA, 4/1 (4 mL) and stirred for 2 h until the product was predominant in UPLC analysis. The mixture was then concentrated and the crude was directly purified by preparative HPLC to give the desired product LC475 after lyophilization.

### LC471F1

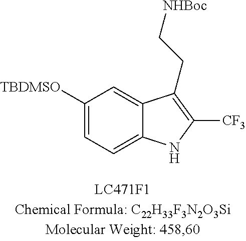 [SMILES: CC(C)(C)OC(=O)NCCc1c(C(F)(F)F)[nH]c2ccc(O[Si](C)(C)C(C)(C)C)cc12]

Yield: 59 mg, 22%. Isolated as a light grey powder, >95% pure by NMR and a single spot by TLC; R_{f}: 0.64 in n-Hex/EtOAc, 80/20. Strain green with ninhydrin;

¹H NMR (CD₃OD, 400 MHZ): δ ppm=7.27 (1H, d, J=8.2 Hz); 7.13 (1H, s); 6.84 (1H, dd, J=2.3 Hz, J=8.7 Hz); 3.26 (2H, t, J=6.8 Hz); 2.98 (2H, t, J=6.8 Hz); 1.44 (9H, s); 1.02 (9H, s); 0.21 (6H, s).

¹⁹F NMR (CD₃OD, 376 MHZ): δ ppm=−59.38.

UPLC: R_{T}: 3.21 (classic system)

MS (ESI⁺) m/z [M+Na]⁺ Calcd for C₂₂H₃₄F₃N₂O₃SiNa⁺ 481.22; Found 481.41.

### LC471F2

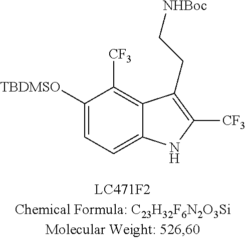 [SMILES: CC(C)(C)OC(=O)NCCc1c(C(F)(F)F)[nH]c2ccc(O[Si](C)(C)C(C)(C)C)c(C(F)(F)F)c12]

Yield: 90 mg, 39%. Isolated as a light grey powder, >95% pure by NMR and a single spot by TLC; R_{f}: 0.53 in n-Hex/EtOAc, 80/20. Strain green with ninhydrin;

¹H NMR (CD₃OD, 400 MHZ): δ ppm=7.55 (1H, d, J=8.7 Hz); 6.96 (1H, d, J=8.7 Hz); 3.21 (2H, t, J=6.4 Hz); 3.05 (2H, t, J=6.4 Hz); 1.42 (9H, s); 1.04 (9H, s); 0.26 (6H, s).

¹⁹F NMR (CD₃OD, 376 MHZ): δ ppm=−52.95; −59.38.

UPLC: R_{T}: 3.20 (classic system)

MS (ESI⁺) m/z [M+Na]⁺ Calcd for C₂₃H₃₂F₆N₂O₃SiNa⁺ 549.21; Found 549.48.

### LC474

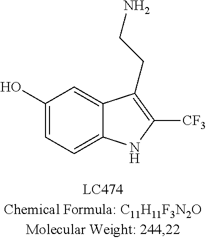 [SMILES: NCCc1c(C(F)(F)F)[nH]c2ccc(O)cc12]

Yield: 22 mg, 52%. Isolated as a light grey powder, >95% pure by NMR;

¹H NMR (CD₃OD, 500 MHz): δ ppm=7.29 (1H, d, J=8.6 Hz); 7.01 (1H, d, J=2.2 Hz); 6.89 (1H, dd, J=2.2 Hz, J=8.7 Hz); 3.19 (2H, m); 3.14 (2H, m).

¹³C NMR (CD₃OD, 126 MHz): δ ppm=152.8; 132.3; 128.7; 124.2 (d, J=38.2 Hz); 123.5 (d, J=268.2 Hz); 116.6; 114.1; 111.1; 103.5; 41.1; 23.2.

¹⁹F NMR (CD₃OD, 470 MHZ): δ ppm=−59.4

UPLC: R_{T}: 1.31 (classic system)

MS (ESI⁺) m/z [M+H]⁺ Calcd for C₁₁H₁₂F₃N₂O⁺ 245.08; Found 245.24.

### LC475

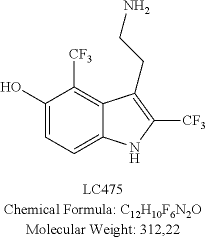 [SMILES: NCCc1c(C(F)(F)F)[nH]c2ccc(O)c(C(F)(F)F)c12]

Yield: 38 mg, 58%. Isolated as a light grey powder, >95% pure by NMR;

¹H NMR (CD₃OD, 500 MHz): δ ppm=8.50 (1H, s, FA); 7.53 (1H, d, J=8.8 Hz); 6.99 (1H, d, J=8.8 Hz); 3.26 (2H, m); 3.06 (2H, m).

¹³C NMR (CD₃OD, 126 MHz): δ ppm=168.2 (FA); 151.9; 131.2; 125.5 (d, J=269.3 Hz); 125.3 (d, J=39.4 Hz); 122.2; 121.9 (d, J=268.2 Hz); 117.7; 116.3; 109.8; 104.8 (d, J=31.2 Hz); 39.9; 22.9.

¹⁹F NMR (CD₃OD, 470 MHz): δ ppm=−53.5; −59.6.

UPLC: R_{T}: 1.43 (classic system)

MS (ESI⁺) m/z [M+H]⁺ Calcd for C₁₂H₁₀F₆N₂O⁺ 313.07; Found 313.16.

### 3-7—A3/Pyrrole C-Trifluoromethylation

### Strategy

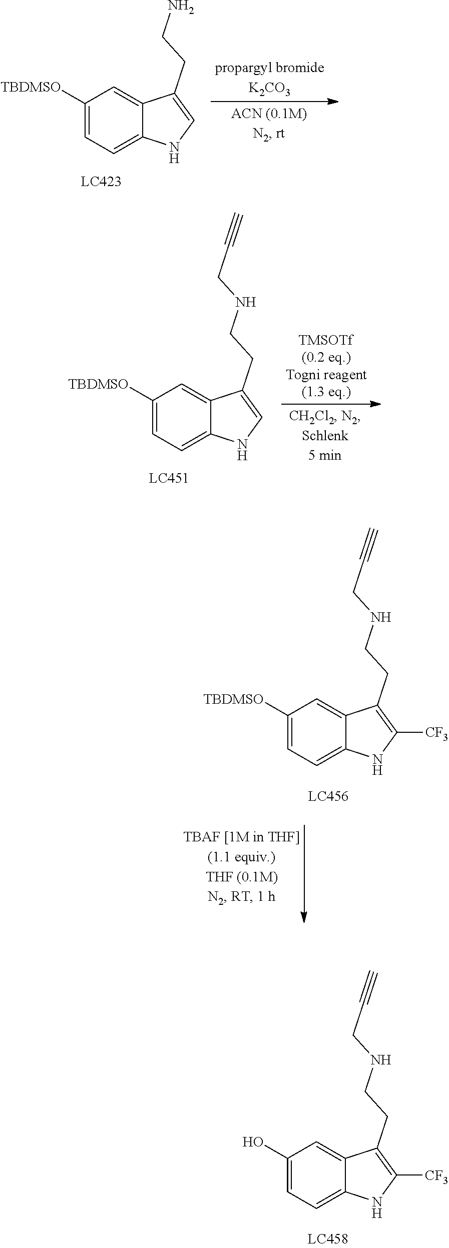 [SMILES: CC(C)(C)[Si](C)(C)Oc1ccc2[nH]cc(CCN)c2c1 ; C#CCNCCc1c[nH]c2ccc(O[Si](C)(C)C(C)(C)C)cc12 ; C#CCNCCc1c(C(F)(F)F)[nH]c2ccc(O[Si](C)(C)C(C)(C)C)cc12 ; C#CCNCCc1c(C(F)(F)F)[nH]c2ccc(O)cc12]

In the dark and under inert atmosphere, LC423 (145 mg, 0.5 mmol, 1 equiv.) was dissolved in dry acetonitrile (5 mL). K₂CO₃ (83 mg, 1.2 equiv.) and propargyl bromide (56 μL, 1 equiv.) were added to the solution. The mixture was stirred and heated at reflux overnight, cooled down to r.t. then filtered with acetonitrile and concentrated. The crude was purified by flash chromatography using DCM/MeOH (100/0 to 80/20) as eluent to give LC451.

For the trifluoromethylation of indole, LC451 (67 mg, 0.2 mmol) and Togni reagent (140 mg, 1.3 equiv.) were added to a Schlenk tube. Under vacuum, DCM (2 mL) and TMSOTf (7.4 μL, 0.2 equiv.) were added and the mixture was stirred for 15 min. NaHCO₃ aq. sat. (15 mL) was added to quench the reaction mixture, then extracted with CH₂Cl₂ (3×20 mL). The combined CH₂Cl₂ extracts were dried over Mg₂SO₄. The crude was purified by flash chromatography using n-Hex/EtOAc (100/0 to 50/50) as eluent to give the desired product LC456 as a white solid.

LC456 was dissolved in THF (C=0.1M) and 1.1 equivalent of TBAF (1 M in THF) was added. The mixture was stirred for 1 h until the O-deprotected product was predominant in UPLC analysis. The mixture was then concentrated and the crude was directly purified by preparative HPLC to give the desired product LC458 after lyophilization.

### LC451

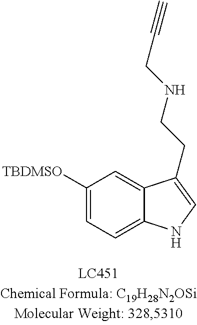 [SMILES: C#CCNCCc1c[nH]c2ccc(O[Si](C)(C)C(C)(C)C)cc12]

Yield: 67 mg, 41%. Isolated as a light grey powder, >95% pure by NMR and a single spot by TLC; R_{f}: 0.67 in DCM/MeOH, 90/10. Strain green with ninhydrin;

¹H NMR (CD₃OD, 400 MHZ): δ ppm=7.19 (1H, dd, J=0.6 Hz, J=8.6 Hz); 7.04 (1H, s); 6.98 (1H, dd, J=0.6 Hz, J=2.4 Hz); 6.67 (1H, dd, J=2.3 Hz, J=8.5 Hz); 3.41 (2H, d, J=2.5 Hz); 2.97 (2H, m); 2.89 (2H, m); 2.56 (1H, t, J=2.5 Hz); 1.01 (9H, s); 0.19 (6H, s).

¹³C NMR (CD₃OD, 101 MHZ): δ ppm=149.7; 134.0; 129.2; 124.4; 116.6; 112.9; 112.6; 108.9; 81.9; 73.2; 49.8; 38.3; 26.3 (3C); 26.0; 19.1; −4.2 (2C).

UPLC: R_{T}: 2.10 (classic system)

MS (ESI⁺) m/z [M+H]⁺ Calcd for C₁₉H₂₈N₂OSi⁺ 329.20; Found 328.98.

### LC456

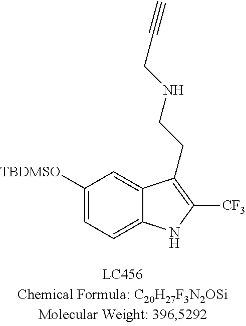 [SMILES: C#CCNCCc1c(C(F)(F)F)[nH]c2ccc(O[Si](C)(C)C(C)(C)C)cc12]

Yield: 19 mg, 23%. Isolated as a light grey powder, >95% pure by NMR and a single spot by TLC; R_{f}: 0.6 in n-Hex/EtOAc, 60/40. Strain green with ninhydrin;

¹H NMR (CD₃OD, 400 MHZ): δ ppm=7.30 (1H, d, J=8.7 Hz); 7.09 (1H, d, J=2.6 Hz); 6.86 (1H, dd, J=2.4 Hz, J=8.7 Hz); 3.59 (2H, d, J=2.3 Hz); 3.08 (2H, m); 3.02 (2H, m); 2.76 (1H, t, J=2.4 Hz); 1.02 (9H, s); 0.21 (6H, s).

UPLC: R_{T}: 2.23 (classic system)

MS (ESI⁺) m/z [M+H]⁺ Calcd for C₂₀H₂₈F₃N₂OSi⁺ 397.18; Found 397.40.

### LC458 (=Compound (036))

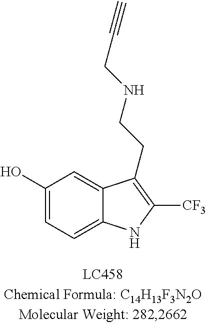 [SMILES: C#CCNCCc1c(C(F)(F)F)[nH]c2ccc(O)cc12]

Yield: 7 mg, 63%. Isolated as a light grey powder, >95% pure by NMR;

¹H NMR (CD₃OD, 500 MHZ): δ ppm=8.41 (1H, s, FA); 7.28 (1H, d, J=8.7 Hz); 7.01 (1H, d, J=2.0 Hz); 6.88 (1H, dd, J=2.0 Hz, J=8.7 Hz); 3.82 (2H, d, J=2.2 Hz); 3.18 (4H, m); 3.05 (1H, t, J=2.1 Hz).

¹⁹F NMR (CD₃OD, 470 MHz): δ ppm=−59.41.

UPLC: R_{T}: 1.45 (classic system)

MS (ESI⁺) m/z [M+H]⁺ Calcd for C₁₄H₁₄F₃N₂OSi⁺ 283.10; Found 283.12.

### 3-8—Bromination

### Strategy

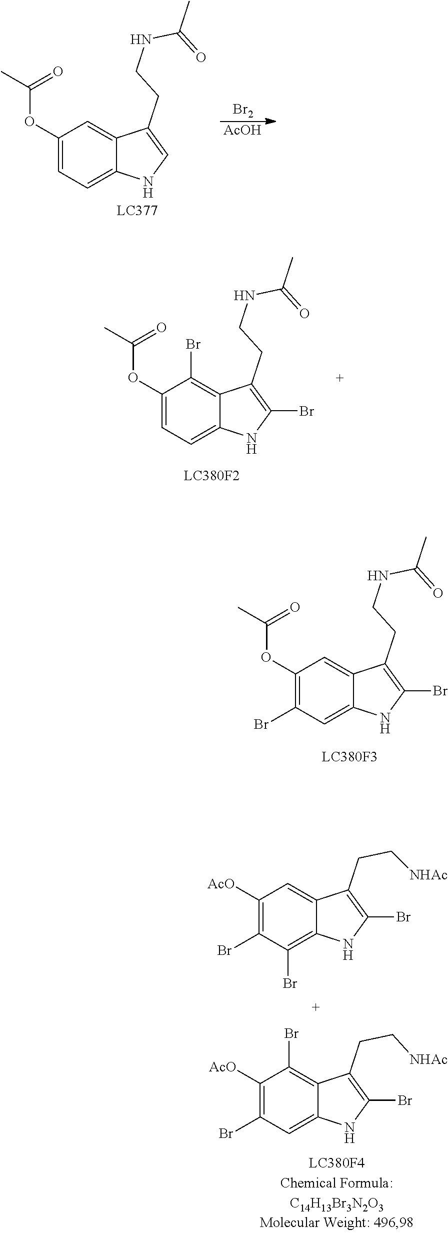

To a cold solution (0° C.) of diacetylated serotonin derivative LC377 (284 mg, 1.1 mmol) in AcOH (11 mL) was added dropwise bromide (67 μL, 1.2 equiv.) over 5 min. After the addition was complete, the reaction mixture was allowed to warm at room temperature and stirred for 5 hours. The reaction was poured in cold water (30 mL) and extracted with EtOAc (3×30 mL), dried over MgSO₄ and the solvent was evaporated under reduced pressure. The crude was directly purified by preparative HPLC to give the desired products LC380F2 and LC380F3 after lyophilization.

### LC380F2 (=Compound (051))

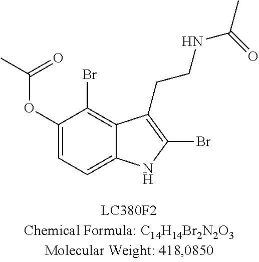 [SMILES: CC(=O)NCCc1c(Br)[nH]c2ccc(OC(C)=O)c(Br)c12]

Yield: 7 mg, 1.5%. Isolated as a light yellow foam, >95% pure by NMR and a single spot by TLC; R_{f}: 0.5 in DCM/MeOH, 95/5. Strain green with CAM;

¹H NMR (CD₃OD, 500 MHZ): δ ppm=8.15 (1H, s, FA); 7.29 (1H, d, J=8.7 Hz); 6.92 (1H, d, J=8.7 Hz); 3.47 (2H, t, J=6.9 Hz); 3.18 (2H, t, J=7.0 Hz); 2.35 (3H, s); 1.92 (3H, s).

¹³C NMR (CD₃OD, 126 MHZ): δ ppm=171.9; 169.7; 141.9; 135.4; 125.7; 116.7; 112.8; 112.3; 110.2; 105.6; 40.5; 24.7; 21.3; 19.3.

### UPLC: R_{T}: 1.96 (Classic System)

MS (ESI⁺) m/z [M+H]⁺ Calcd for C₁₄H₁₅Br₂N₂O₃⁺ 419.05; Found 419.02.

### LC380F3 (=Compound (052))

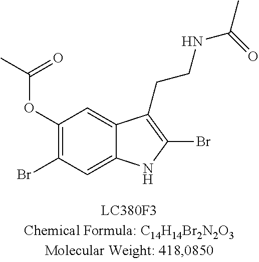 [SMILES: CC(=O)NCCc1c(Br)[nH]c2cc(Br)c(OC(C)=O)cc12]

Yield: 20 mg, 4.4%. Isolated as a light yellow foam, >95% pure by NMR and a single spot by TLC; R_{f}: 0.5 in DCM/MeOH, 95/5. Strain green with CAM;

¹H NMR (CD₃OD, 500 MHZ): δ ppm=7.55 (1H, s); 7.35 (1H, s); 3.38 (2H, t, J=6.9 Hz); 2.88 (2H, t, J=7.0 Hz); 2.35 (3H, s); 1.88 (3H, s).

¹³C NMR (CD₃OD, 126 MHZ): δ ppm=172.1; 169.9; 141.3; 134.9; 127.4; 114.2; 112.0; 111.6; 110.7; 109.1; 39.1; 24.1; 21.2; 19.3.

UPLC: R_{T}: 2.01 (classic system)

MS (ESI⁺) m/z [M+H]⁺ Calcd for C₁₄H₁₅Br₂N₂O₃⁺ 419.05; Found 419.02.

### LC380F4 (=Compound (060))

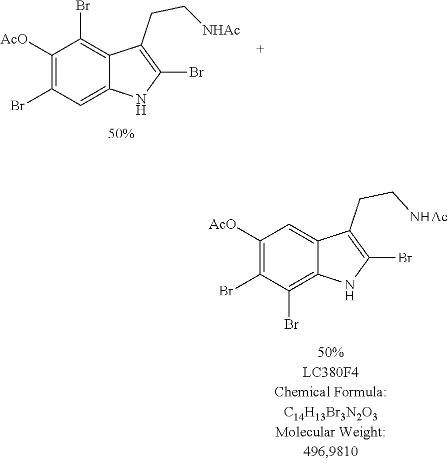 [SMILES: CC(=O)NCCc1c(Br)[nH]c2cc(Br)c(OC(C)=O)c(Br)c12 ; CC(=O)NCCc1c(Br)[nH]c2c(Br)c(Br)c(OC(C)=O)cc12]

Yield: 4 mg, 0.7%. Isolated as a light yellow foam, >95% pure by NMR and a single spot by TLC; R_{f}: 0.5 in DCM/MeOH, 95/5. Strain green with CAM;

¹H NMR (CD₃OD, 500 MHz): Mixture of two isomers δ ppm=7.38 (1H, s); 7.19 (1H, s); 3.47 (2H, t, J=7.0 Hz); 3.39 (2H, t, J=6.8 Hz); 3.18 (2H, t, J=6.7 Hz); 2.89 (2H, t, J=6.7 Hz); 2.35 (6H, s); 1.91 (3H, s); 1.87 (3H, s).

¹³C NMR (CD₃OD, 126 MHZ): Mixture of two isomers δ ppm=172.1; 171.9; 169.6; 169.3; 142.1; 142.0; 134.3; 134.1; 127.2; 126.2; 119.5; 114.4; 113.9; 113.5; 112.3; 112.0; 111.0; 110.7; 106.4; 105.4; 102.4; 40.3; 38.9; 24.9; 24.2; 21.3; 21.2; 19.3; 19.2.

UPLC: R_{T}: 2.05 (classic system)

MS (ESI⁺) m/z [M+H]⁺ Calcd for C₁₄H₁₄Br₃N₂O₃⁺ 494.85; Found 494.72.

### 3-9-Melatonin and Derivatives

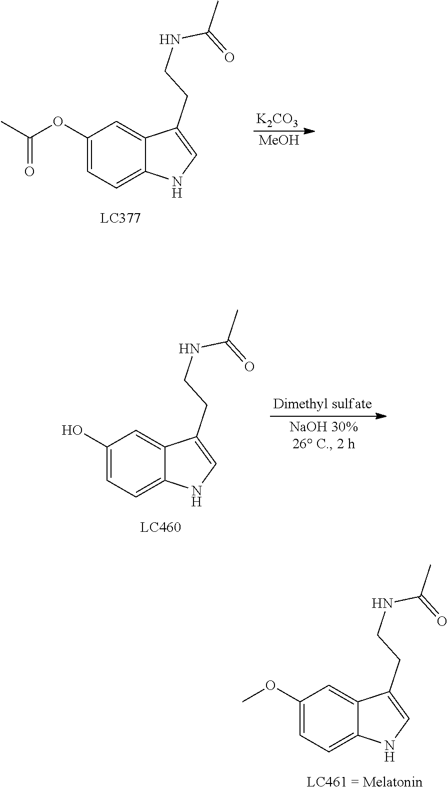

The synthetic procedure is adapted from the patent WO 2011/047156 PCT/US2010/052674. A suspension of LC377 (260 mg, 1 mmol) and K₂CO₃ (138 mg, 1 equiv.) in MeOH (3.2 mL) was stirred at 26° C. for 2 hours. The reaction mixture was concentrated under reduced pressure and then the crude material was diluted in water (10 mL) and extracted with EtOAc (2×20 mL). The combined organic layers were washed with water (10 mL) and brine (10 mL), dried over MgSO₄, and the solvent was evaporated under reduced pressure. The desired compound LC460 was obtained as a pale-yellow foam.

LC460 (117 mg, 0.54 mmol) was solubilized in a 30% NaOH solution (67 μL) and stirred at 26° C. Dimethyl sulfate (86 μL, 1.7 equiv.) was added drop by drop, and the reaction was stirred for 2 hours. The reaction mixture was acidified with 2N HCl to obtain pH=2, then diluted with water (10 mL), and extracted with EtOAc (2×20 mL). The combined organic layers were washed with water (2×10 mL) and brine (10 mL), dried over MgSO₄, and the solvent was removed to afford the crude Melatonin LC461. Melatonin was then purified by preparative HPLC to afford a light-yellow gum.

### LC460

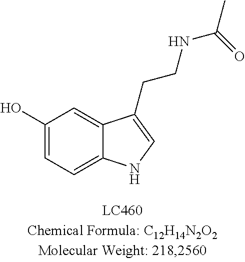 [SMILES: CC(=O)NCCc1c[nH]c2ccc(O)cc12]

Yield: 139 mg, 61%. Isolated as a light yellow foam, >95% pure by NMR and a single spot by TLC; R_{f}: 0.2 in DCM/MeOH, 95/5. Strain green with CAM;

¹H NMR (CD₃OD, 400 MHz): δ ppm=7.22 (1H, dd, J=0.5 Hz, J=8.5 Hz); 7.00 (1H, s); 6.92 (1H, dd, J=0.5 Hz, J=2.5 Hz); 6.65 (1H, dd, J=2.3 Hz, J=8.6 Hz); 3.43 (2H, t, J=7.2 Hz); 2.86 (2H, dt, J=0.6 Hz, J=7.2 Hz); 1.91 (3H, s).

¹³C NMR (CD₃OD, 101 MHz): δ ppm=173.3; 151.1; 133.1; 129.5; 124.2; 112.6; 112.5; 112.3; 103.5; 41.5; 26.3; 22.6.

UPLC: R_{T}: 1.32 (classic system)

MS (ESI⁺) m/z [M+H]⁺ Calcd for C₁₂H₁₅N₂O₂⁺ 219.11; Found 219.12.

### LC461=Melatonin (=Compound (054)

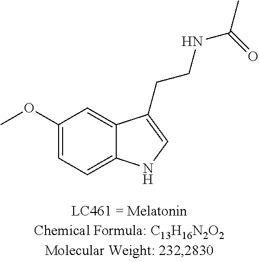 [SMILES: COc1ccc2[nH]cc(CCNC(C)=O)c2c1]

Yield: 76 mg, 61%. Isolated as a light yellow foam, >95% pure by NMR and a single spot by TLC; R_{f}: 0.5 in DCM/MeOH, 95/5. Strain green with CAM;

¹H NMR (CD₃OD, 400 MHZ): δ ppm=7.21 (1H, dd, J=0.4 Hz, J=8.7 Hz); 7.05 (1H, d, J=2.5 Hz); 7.03 (1H, s); 6.75 (1H, dd, J=2.5 Hz, J=8.7 Hz); 3.82 (3H, s); 3.45 (2H, t, J=7.5 Hz); 2.90 (2H, dt, J=0.5 Hz, J=7.5 Hz); 1.91 (3H, s).

¹³C NMR (CD₃OD, 101 MHZ): δ ppm=173.3; 154.9; 133.4; 129.1; 124.1; 113.1; 112.9; 112.6; 101.3; 56.3; 41.5; 26.2; 22.6.

UPLC: R_{T}: 1.68 (classic system)

MS (ESI⁺) m/z [M+H]⁺ Calcd for C₁₃H₁₇N₂O₂+233.12; Found 233.12.

### 3-10—Phenylcarbamate

### Strategy:

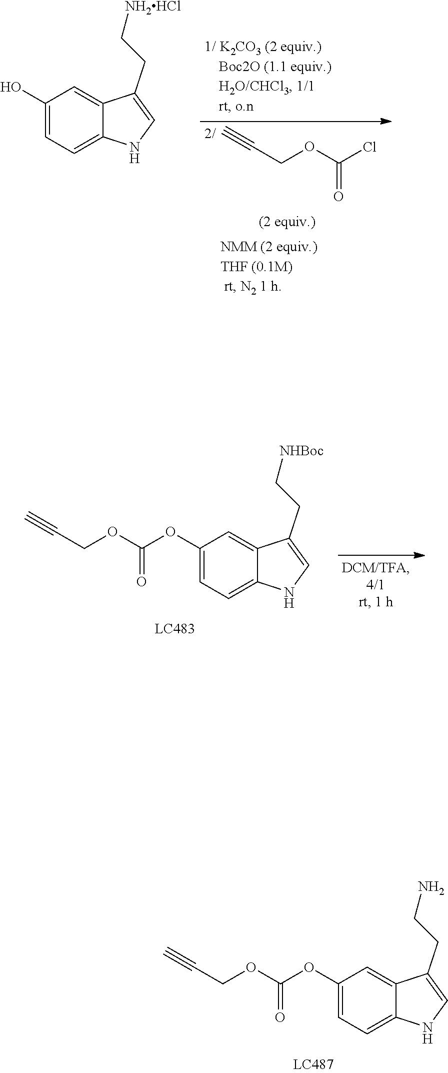

In the dark, serotonin hydrochloride (500 mg, 2.35 mmol, 1 equiv.) was dissolved in water (9 mL). K₂CO₃ (665 mg, 4.81 mmol, 2.1 equiv.) and Boc₂O (538 mg, 2.46 mmol, 1.05 equiv.) were added to the solution. The solution was stirred overnight, and then extracted with DCM. The organic phase was washed with aq. HCl 5% and brine, then dried on MgSO4 and concentrated. The crude was purified by flash chromatography using DCM/MeOH (100/0 to 90/10) as eluent to give the desired product (485 mg), which was engaged in the next step.

In the dark and under inert atmosphere, the product of the previous step (138 mg, 0.5 mmol, 1 equiv.) was dissolved in dry THF (4 mL). NMM (110 μL, 2 equiv.) and propargylchloroformate (97 μL, 2 equiv.) were added to the solution. The mixture was stirred at room temperature for 1 h. The reactional mixture was then concentrated to dryness. The crude was purified by flash chromatography using n-Hex/EtOAC (100/0 to 50/50) as eluent to give LC483.

The product was then dissolved in CH₂Cl₂/TFA, 4/1 (4 mL) and stirred for 2 h until the product was predominant in UPLC analysis. The mixture was then concentrated and the crude was directly purified by preparative HPLC to give the desired product LC487 after lyophilization.

### LC483 (=Compound (055))

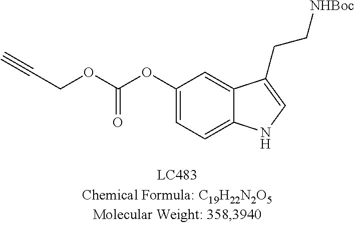 [SMILES: C#CCOC(=O)Oc1ccc2[nH]cc(CCNC(=O)OC(C)(C)C)c2c1]

Yield: 125 mg, 69%. Isolated as a light grey powder, >95% pure by NMR and a single spot by TLC; R_{f}: 0.58 in n-Hex/EtOAc, 60/40. Strain green with CAM

¹H NMR (CDCl₂, 400 MHZ): δ ppm=8.34 (1H, s); 7.36 (2H, m); 7.10 (1H, d, J=1.1 Hz); 6.98 (1H, dd, J=2.3 Hz, J=8.7 Hz); 4.84 (2H, d, J=2.6 Hz); 4.65 (1H, brs); 3.39 (2H, q, J=6.6 Hz); 2.90 (2H, t, J=7.1 Hz); 2.65 (1H, t, J=2.3 Hz); 1.41 (9H, s).

¹³C NMR (CDCl₂, 101 MHZ): δ ppm=156.2; 154.4; 145.1; 134.7; 128.1; 124.4; 115.9; 114.0; 112.0; 110.8; 79.2; 77.3; 76.1; 56.1; 41.2; 28.5 (3C); 26.1.

UPLC: R_{T}: 2.36 (classic system)

MS (ESI⁺) m/z [M+Na]⁺ Calcd for C₁₉H₂₃N₂O₅Na⁺ 381.15; Found 381.29.

### LC487 (=Compound (056))

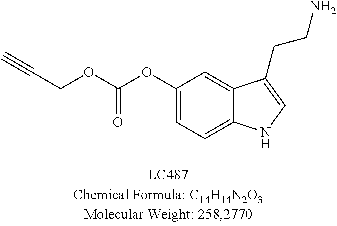 [SMILES: C#CCOC(=O)Oc1ccc2[nH]cc(CCN)c2c1]

Yield: 75 mg, 87%. Isolated as a light grey powder, >95% pure by NMR;

¹H NMR (CD₃OD, 500 MHZ): δ ppm=7.38 (2H, m); 7.24 (1H, s); 6.95 (1H, dd, J=2.3 Hz, J=8.6 Hz); 4.86 (2H, d, J=2.6 Hz); 3.19 (2H, t, J=7.0 Hz); 3.07 (3H, m).

¹³C NMR (CD₃OD, 126 MHZ): δ ppm=155.7; 145.9; 136.2; 128.3; 126.3; 116.2; 113.1; 110.8; 110.7; 78.1; 77.3; 56.7; 41.1; 24.3.

UPLC: R_{T}: 1.41 (classic system)

MS (ESI⁺) m/z [M+H]⁺ Calcd for C₁₄H₁₅N₂O₅⁺ 259.10; Found 259.10.

### LT273 (=Compound (057))

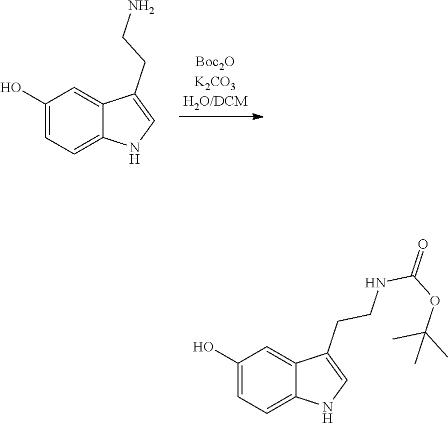

In the dark, serotonin hydrochloride (500 mg, 2.35 mmol, 1 equiv.) was dissolved in water (9 mL). K₂CO₃ (665 mg, 4.81 mmol, 2.1 equiv.) and Boc₂O (538 mg, 2.46 mmol, 1.05 equiv.) were added to the solution. The solution was stirred overnight, and then extracted with DCM. The organic phase was washed with aq. HCl 5% and brine, then dried on MgSO4 and concentrated. The crude was purified by flash chromatography using DCM/MeOH (100/0 to 90/10) as eluent to give the desired product (quantitative yield). Then it was further purified by HPLC.

¹H NMR (, CD₂Cl₂, 500 MHz) δ ppm=8.12 (s, 1H), 7.26 (dd, J=8.6, 0.6 Hz, 1H), 7.05 (d, J=2.3 Hz, 1H), 7.03 (d, J=2.4 Hz, 1H), 6.79 (dd, J=8.7, 2.4 Hz, 1H), 4.76 (s, 1H), 3.43 (d, J=6.9 Hz, 2H), 2.89 (t, J=7.0 Hz, 2H), 1.46 (s, 9H).

¹³C NMR (, CD₂Cl₂, 126 MHz) δ ppm=155.98, 149.66, 131.58, 128.15, 123.21, 112.37, 111.74, 103.11, 78.95, 40.77, 28.13, 25.82.

MS (ESI⁺) m/z [M+H]⁺ Calcd for C₁₅H₂₁N₂O₃⁺ 277.14; found 277.24.

### LT282 (=Compound (061))

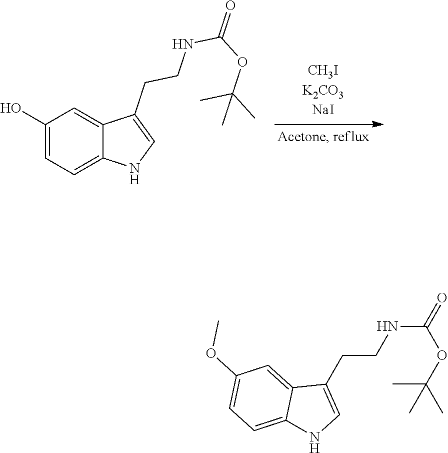 [SMILES: CC(C)(C)OC(=O)NCCc1c[nH]c2ccc(O)cc12 ; COc1ccc2[nH]cc(CCNC(=O)OC(C)(C)C)c2c1]

To solution of LT273 (200 mg) in acetone anhydrous (8 mL), K₂CO₃ (300 mg, 3 eq) and NaI (109 mg, 1 eq) were added and stirred at room temperature for 5 minutes. Then, CH₃I was added to the reaction mixture and refluxed for 5 hours. The solution was concentrated in vacuo and purified by flash chromatography in EtOAc and cyclohexane gradient. It was then further purified by HPLC to obtain pure final product (80 mg, 38%).

¹H NMR (MeOD, 500 MHz) δ ppm=7.23 (d, J=8.7 Hz, 1H), 7.08 (s, 1H), 7.03 (s, 1H), 6.76 (dt, J=8.7, 1.9 Hz, 1H), 3.84 (s, 3H), 3.33 (t, J=7.4 Hz, 2H), 2.88 (t, J=7.4 Hz, 2H), 1.44 (s, 9H).

¹³C NMR (MeOD, 126 MHz) δ ppm=157.12, 153.54, 132.00, 127.74, 122.75, 111.84, 111.43, 111.14, 100.05, 78.50, 54.96, 41.04, 27.39, 25.53.

MS (ESI⁺) m/z [M+H]⁺ Calcd for C₁₆H₂₃N₂O₃⁺ 291.16; found 291.32.

### LT299F1

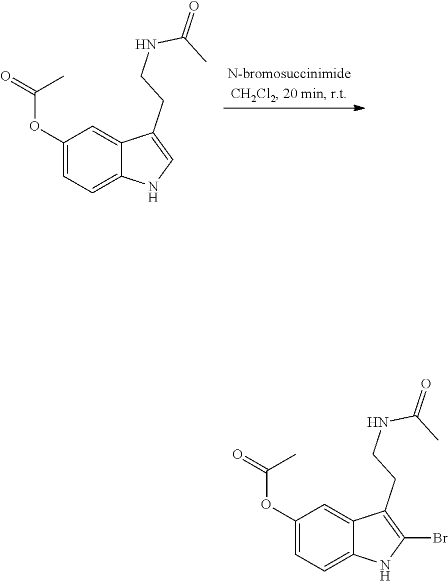 [SMILES: CC(=O)NCCc1c[nH]c2ccc(OC(C)=O)cc12 ; CC(=O)NCCc1c(Br)[nH]c2ccc(OC(C)=O)cc12]

To diacetylated serotonin derivative LC337 (200 mg) in anhydrous DCM (4 mL), 0.6 eq of N-bromosuccinimide (96 mg) was added and stirred at room temperature for 20 minutes. Then 0.1 eq of N-bromosuccinimide (15 mg) was added further and left for another 20 minutes. The reaction solution was concentrated in vacuo and purified by flash chromatography in EtOAc and cyclohexane gradient and then finally by HPLC to obtain the final product (13 mg, 5%).

¹H NMR (CD₃CN, 500 MHz) δ ppm=9.64 (s, 1H), 7.35 (dd, J=8.7, 0.6 Hz, 1H), 7.28-7.19 (m, 1H), 6.89 (dd, J=8.7, 2.2 Hz, 1H), 6.42 (s, 1H), 3.35 (td, J=6.9, 6.1 Hz, 2H), 2.83 (t, J=6.9 Hz, 2H), 2.28 (s, 3H), 1.81 (s, 3H).

¹³C NMR (CD₃CN, 126 MHz) δ ppm=170.24, 169.65, 144.63, 134.15, 127.84, 116.35, 112.60, 111.15, 110.33, 109.92, 38.79, 24.73, 22.15, 20.28.

MS (ESI⁺) m/z [M+H]⁺ Calcd for C₁₄H₁₆BrN₂O₃⁺ 339.03; found 339.21

### Example 2: Biological Activity

### Materials and Methods

### Complexation Studies

Calorimetric assessment of complex formation: To an aqueous solution of serotonin hydrochloride (3 equiv., C=0.1 M) or A3 formic salt (3 equiv., C=0.1 M) iron (III) chloride (1 equiv.) or copper (II) sulfate (C=0.1 M) was added. A color change characterized the formation of metal complexes.

For NMR experiments: NMR spectroscopy was performed on a 500 MHz Bruker spectrometer. Samples were dissolved in D₂O or CD₃OD, at 298 K or 310K. ¹H-NMR were recorded at 500 MHz and chemical shifts δ are expressed in ppm using the residual non-deuterated solvent signal as internal standard. Titration of the samples up to 0.3, 0.6 or 1 mol equivalent of iron or copper was performed, using conditions (solvent and temperature) as indicated in the figures. For some samples, trifluoroacetic acid (TFA) was added as indicated in the figures.

### Cell Culture

All cells were grown in an incubator equilibrated at 37° C. with 5% CO₂, grown to confluence and split with Trypsin/EDTA (Gibco, TRYPGIB01) once or twice a week according to confluence. HMLER cells (sex: female) naturally repressing E-cadherin, obtained from human mammary epithelial cells infected with a retrovirus carrying hTERT, SV40 and the oncogenic allele H-rasV12, and HMLER CD44 and TFRC ko clones were cultured in DMEM/F12 (Thermo Fisher Scientific, 31331093) supplemented with 10% FBS (v/v) (Thermo Fisher Scientific, 10270106), 10 μg/mL insulin (Sigma-Aldrich, 10516), 0.5 μg/mL hydrocortisone (Sigma-Aldrich, H0888) and 0.5 μg/ml puromycin (Life Technologies, A11138-02), unless stated otherwise. PDAC053 T cells were grown in serum-free ductal medium: DMEM/F12 supplemented with 0.61 g/500 mL nicotinamide (Sigma-Aldrich, 3376), 2.50 g/500 mL glucose (Sigma-Aldrich, G6152), 1:200 ITS+ (Corning, 354352), 1:20 Nu-serum IV (Corning, 355104), 100 ng/ml cholera toxin, 1 μM dexamethasone (Sigma-Aldrich, D4902), 50 nM 3,3′,5-triiodo-L-thyronine (Sigma-Aldrich, T6397) and 1× PenStrep. BELA cells were cultured using published procedures (Nat. Comm. doi: 10.1038/ncomms14750).

### Fluorescence Microscopy

Immunofluorescence. Cells were plated on cover slips 24 h prior to the experiment. Subsequently, cells were washed three times with 1×PBS and fixed with 2% paraformaldehyde (w/v) in 1×PBS for 12 min, and then washed three times with 1×PBS. After fixation, cells were permeabilized with 0.1% Triton X-100 (v/v) in 1×PBS for 5 min and washed three times with 1×PBS. Then, cells were blocked in 2% BSA, 0.2% Tween-20 (w/v)/1×PBS (blocking buffer) for 20 min at room temperature. Cells were incubated with the relevant antibody in blocking buffer for 1 h at room temperature and washed three times with blocking buffer. Then, cells were incubated with secondary antibodies for 1 h. Finally, cover slips were washed three times with 1×PBS and mounted using VECTASHIELD (Vector Laboratories) containing DAPI.

Fluorescence microscopy of clickable serotonin analog A3: Cells were plated on cover slips 24 h prior to the experiment. The cells were treated with 10 μM A3 for 3 h and LysoTracker Deep Red (Thermo Fisher Scientific L12492, 1:10000) according to the manufacturer's protocol. Subsequently, cells were washed three times with 1×PBS and fixed with 2% paraformaldehyde (w/v) in 1×PBS for 12 min, and then washed three times with 1×PBS. After fixation, cells were permeabilized with 0.1% Triton X-100 (v/v) in 1×PBS for 5 min and washed three times with 1×PBS. Then, cells were blocked in 2% BSA, 0.2% Tween-20 (w/v)/1×PBS (blocking buffer) for 20 min at RT. The click reaction cocktail was prepared from Click-iT EdU Imaging Kits (ThermoFisher Scientific, C10337) according to the manufacturer's protocol. Briefly, 868 μL of 1× Click-iT reaction buffer was mixed with 40 μL CuSO₄ solution, 2 μL Alexa Fluor azide and 90 μL reaction buffer additive (sodium ascorbate) to reach a final volume of 1 mL. Cover slips were incubated with 50 μl of the click reaction cocktail in the dark at RT for 30 minutes, then washed three times with 1×PBS. Cover slips were mounted using VECTASHIELD containing DAPI and were sealed with nail varnish Express manucure (Maybelline, 16P201).

### Flow Cytometry

Bodipy-C11 (Thermo Fisher Scientific, D3861) were used according to the manufacturer's protocol. RhoNox-M (in-house) was added to live cells prior to flow cytometry analysis for 1 h at a concentration of 1 μM. For the Annexin/PI assay (Thermo Fisher Scientific, V13245, manufacturer's protocol) BELA cells were treated with RSL3 in the presence or absence of 1 μM Liproxstatin-1 (Sigma-Aldrich, SML1414) or 10 μM A3 for 24 h. Cells were then washed twice with ice-cold 1×PBS and suspended in incubation buffer prior to being analyzed by flow cytometry. The manufacturer's protocol was followed. For each condition, at least 10,000 cells were counted. Data were recorded on an Attune (Thermo Fisher Scientific) and processed using FlowJo (FLOWJO, LLC).

### Inductively Coupled Plasma Mass Spectrometry

Glass vials equipped with Teflon septa were cleaned with nitric acid 65% (w/v) (VWR, Suprapur, 1.00441.0250), washed with ultrapure water (Sigma-Aldrich, 1012620500) and dried. Cells were plated 24 h prior to the experiments. Cells were harvested using trypsinization (TrypLE Express Enzyme, Life Technologies, 12605010) followed by two washes with 1×PBS. Cells were then counted using an automated cell counter (Entek) and transferred in 100 μL 1×PBS to the cleaned glass vials, and samples were lyophilized using a freeze dryer (CHRIST, 22080). Samples were subsequently mixed with nitric acid 65% (w/v) overnight followed by heating at 80° C. for 2 h. Samples were diluted with ultrapure water (Sigma-Aldrich, 1012620500) to a final concentration of 0.475 N nitric acid and transferred to metal-free centrifuge vials (VWR, 89049-172) for subsequent ICP-MS analysis. 56Fe concentrations were measured using an Agilent 7900 ICP-QMS in low-resolution mode. Sample introduction was achieved with a micro-nebulizer (MicroMist, 0.2 mL/min) through a Scott spray chamber. Isotopes were measured using a collision-reaction interface with helium gas (5 mL/min) to remove polyatomic interferences. Scandium and indium internal standards were injected after inline mixing with the samples to control the absence of signal drift and matrix effects. A mix of certified standards was measured at concentrations spanning those of the samples to convert count measurements to concentrations in the solution. Uncertainties on sample concentrations were calculated using algebraic propagation of ICP-MS blank and sample counts uncertainties. Values were normalized by dry weight and cell number.

### Cell Viability

Cell viability was evaluated by plating 4,000 cells/well in 96-well plates using CellTiter-Blue viability assay according to the manufacturer's protocol. Cells were treated as indicated in the figure. CellTiter-Blue reagent (Promega, G8081) was added after 72 h treatment and cells were incubated for 2 h before recording fluorescence intensities (Eₑₓ. 560 nm; Eₑₘ. 590 nm) using a Perkin Elmer Wallac 1420 Victor2 Microplate Reader.

### Results

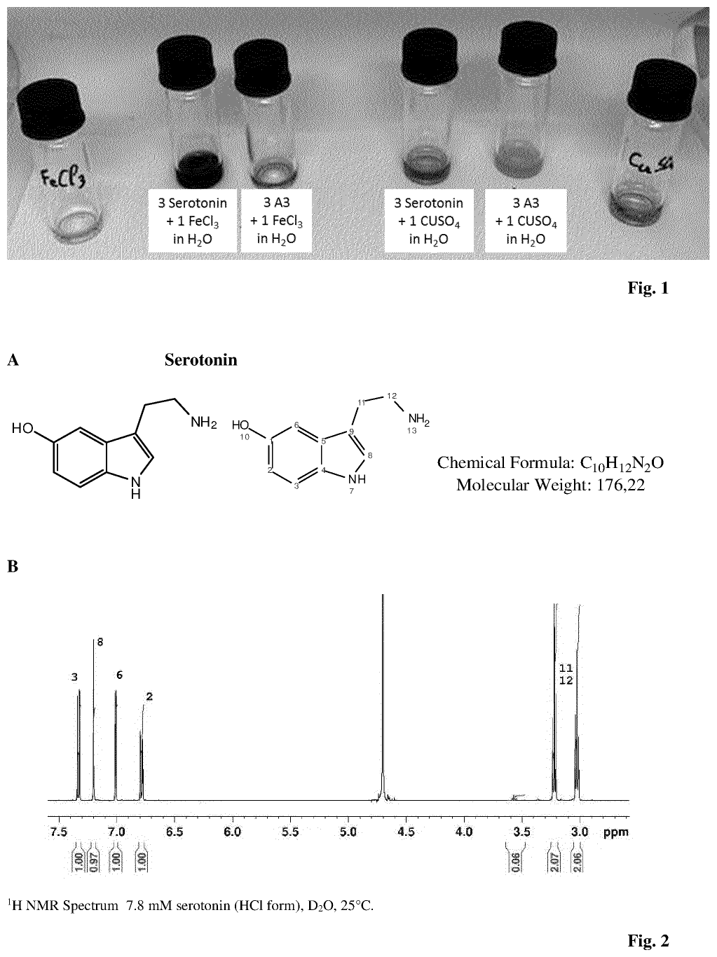

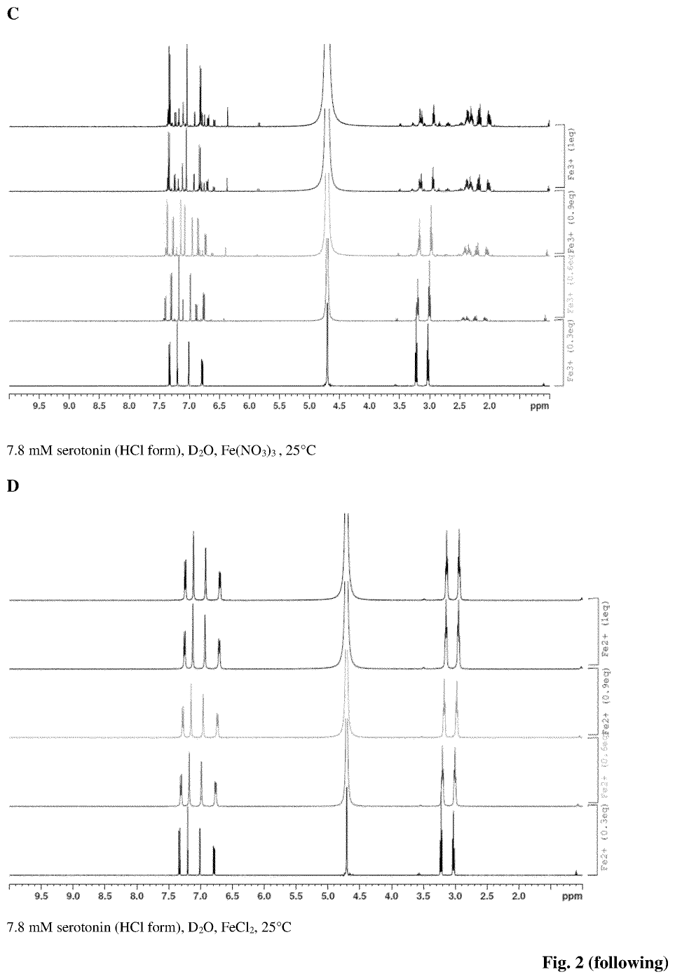

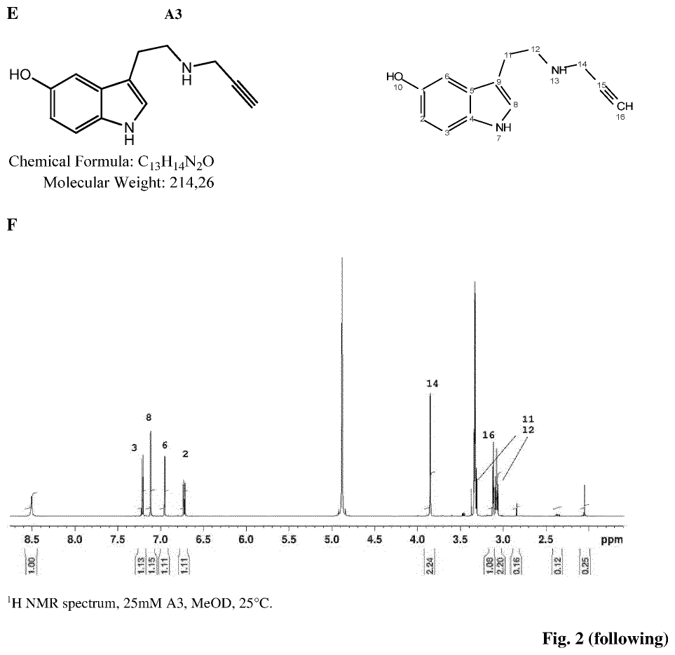

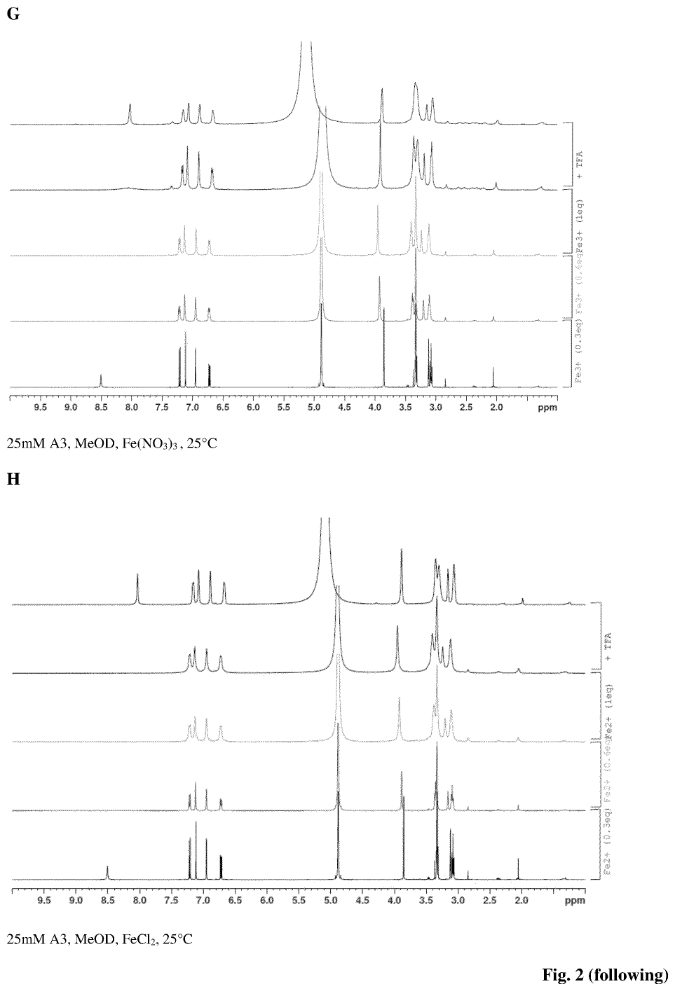

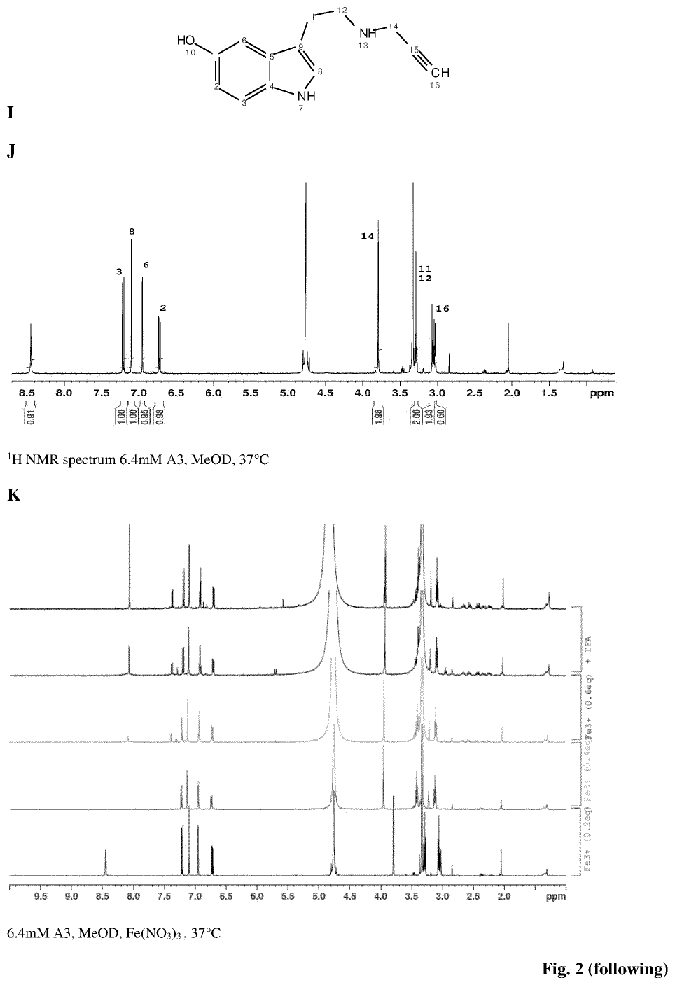

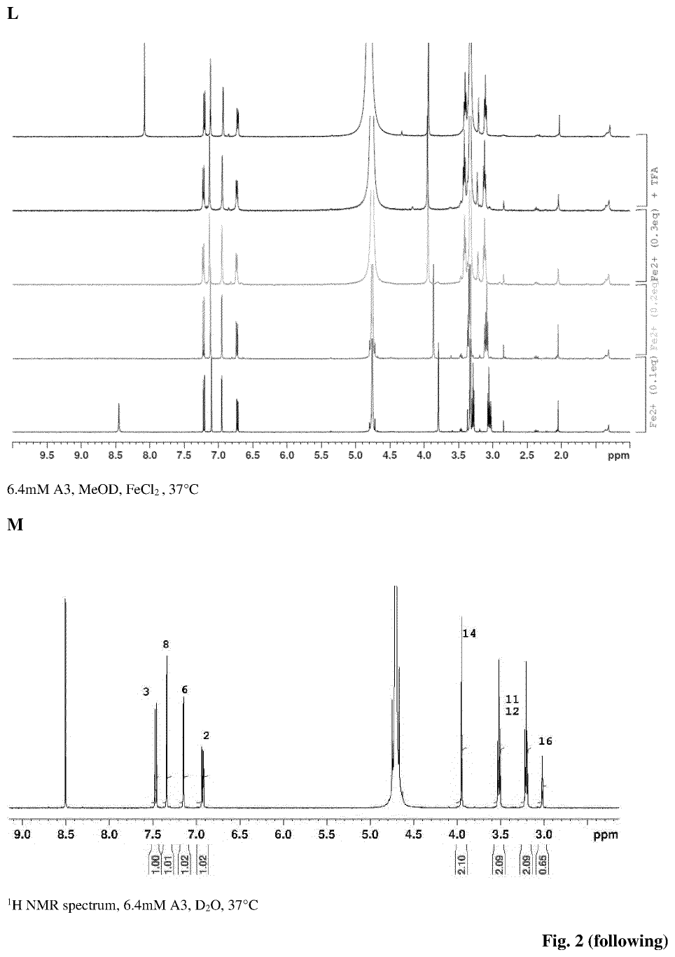

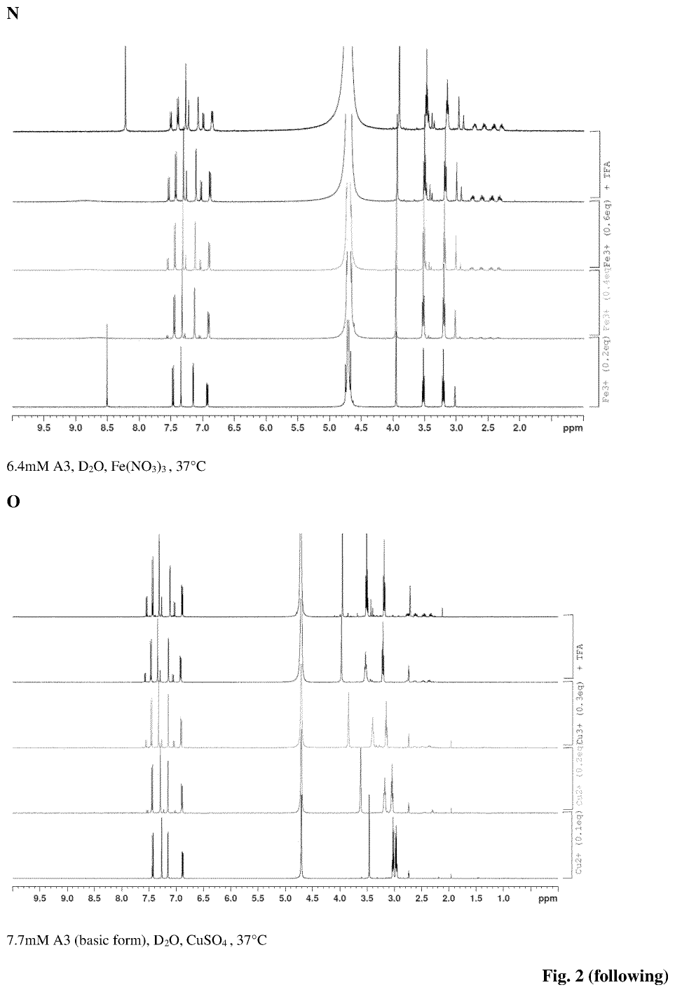

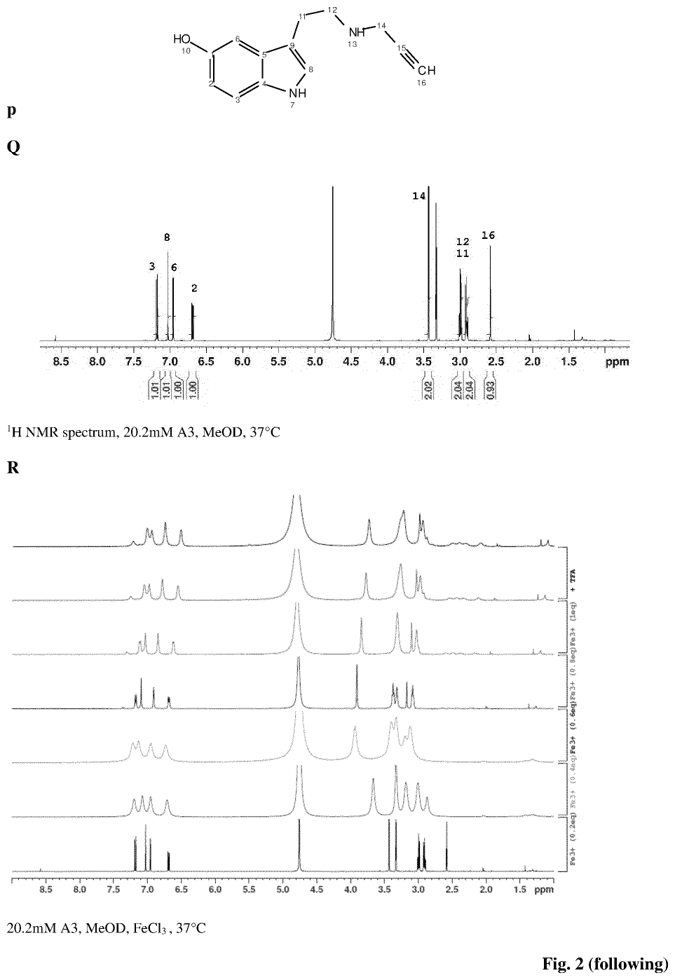

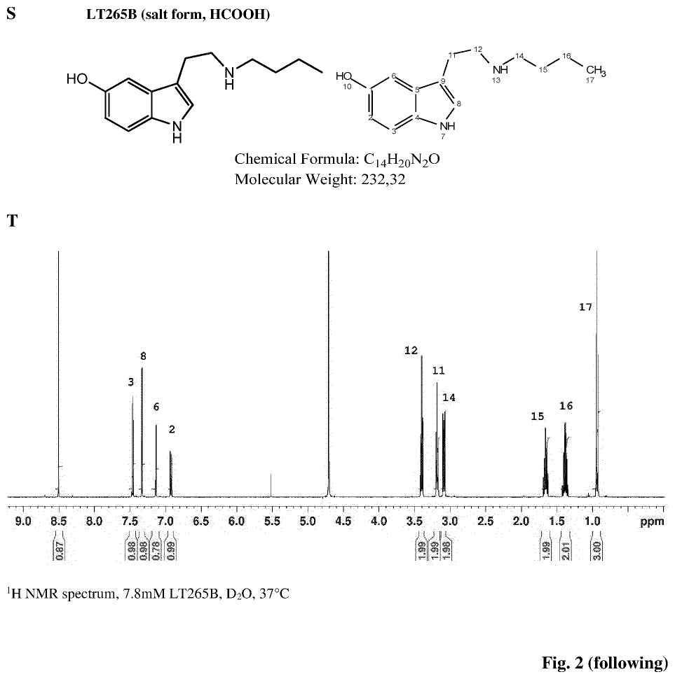

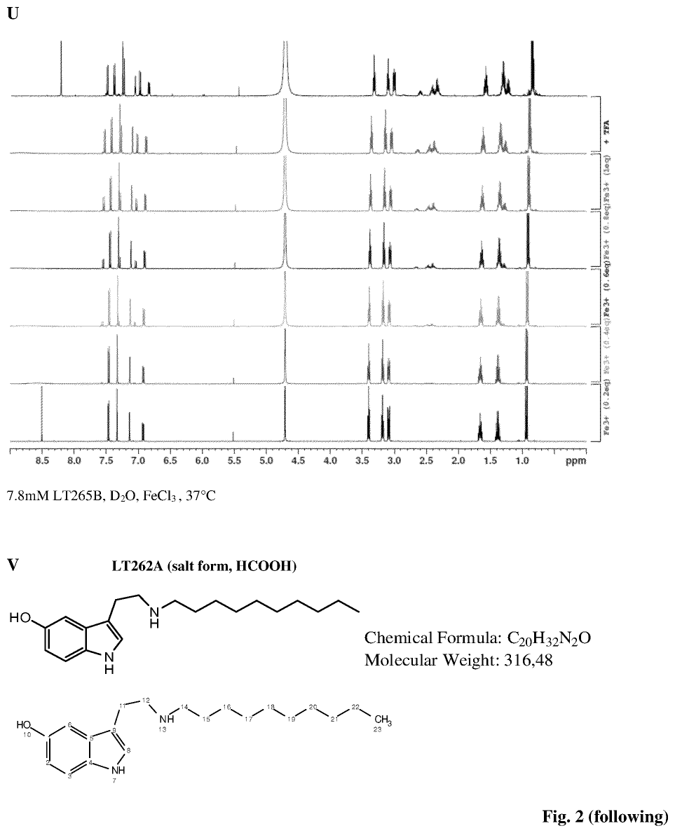

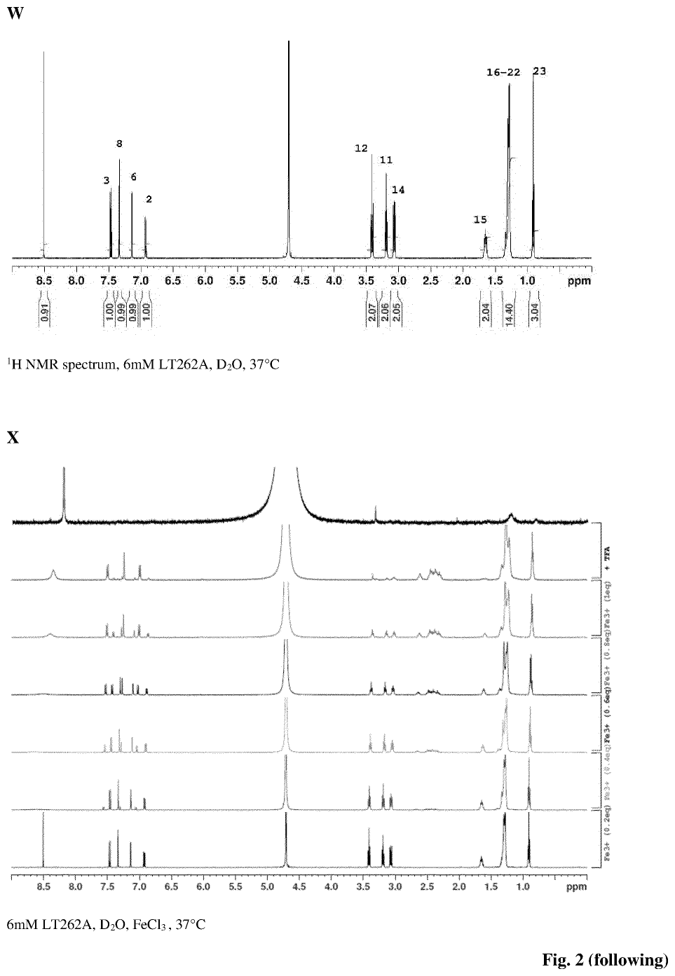

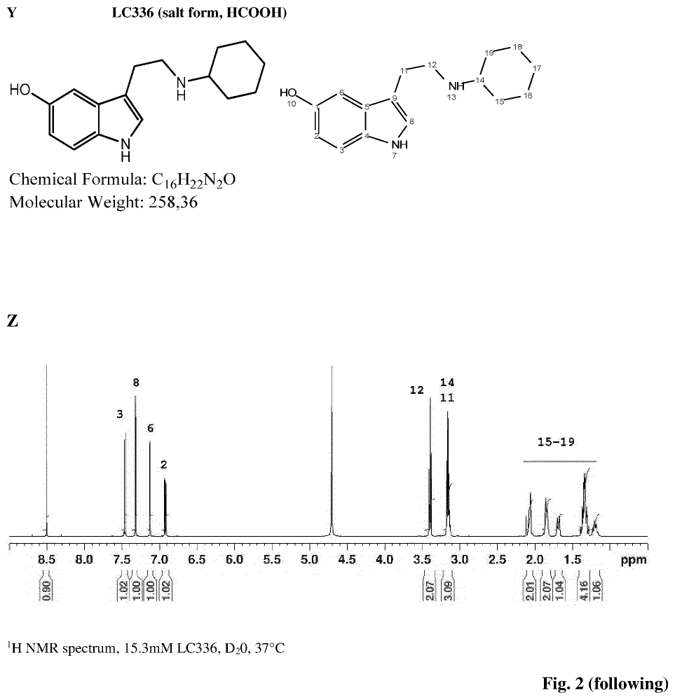

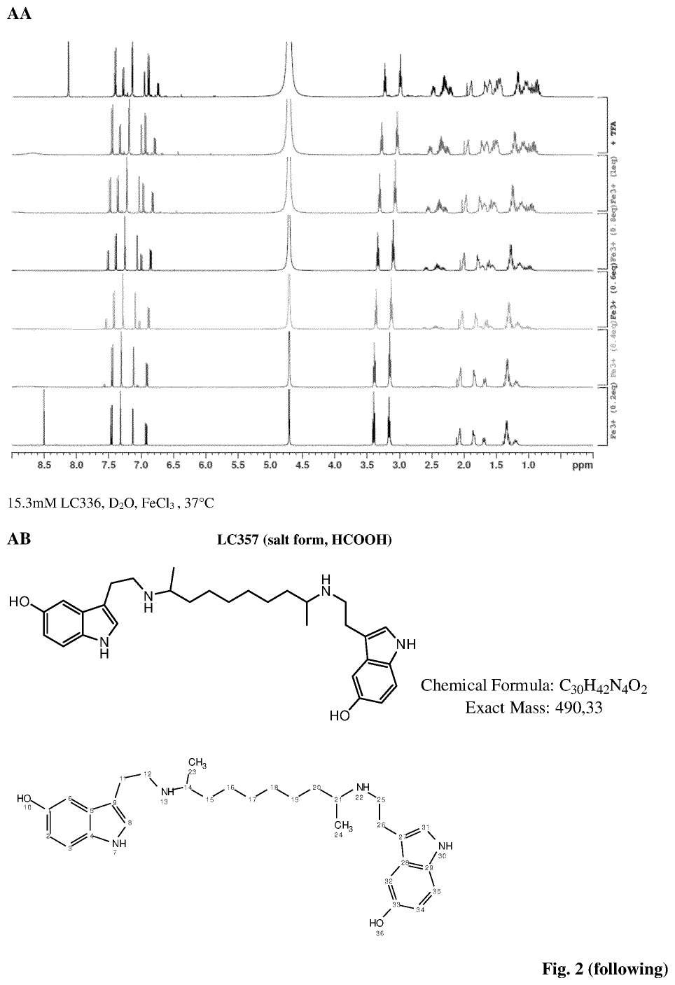

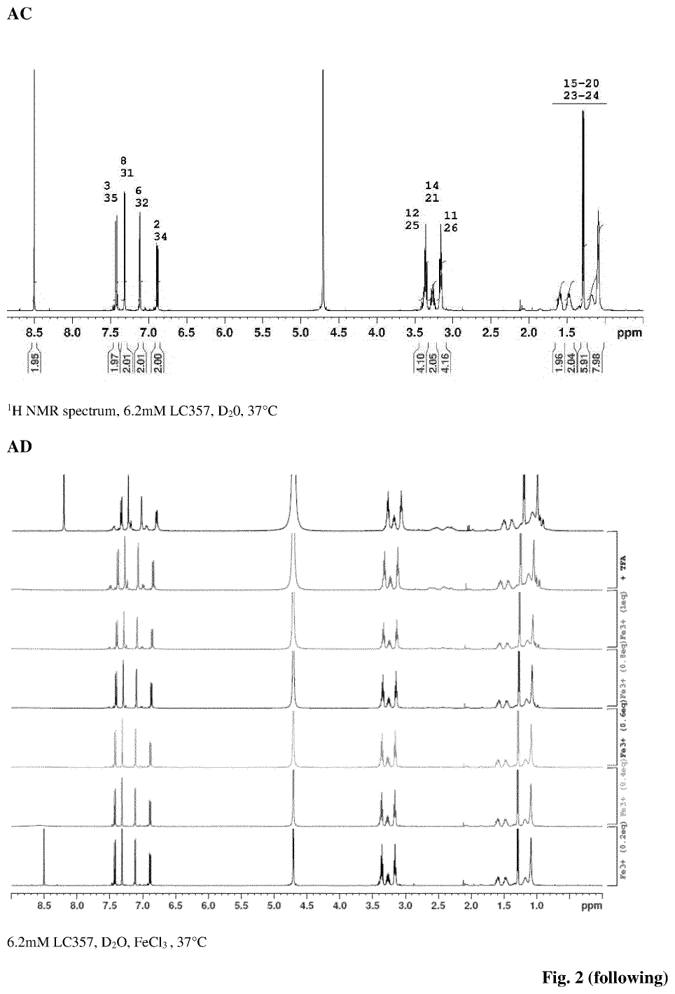

Serotonin and A3 form readily complexes with Fe(II), Fe(III) and Cu(II) as attested by colorimetric complexation studies in solution (FIG. 1) and by NMR spectroscopy (FIG. 2).

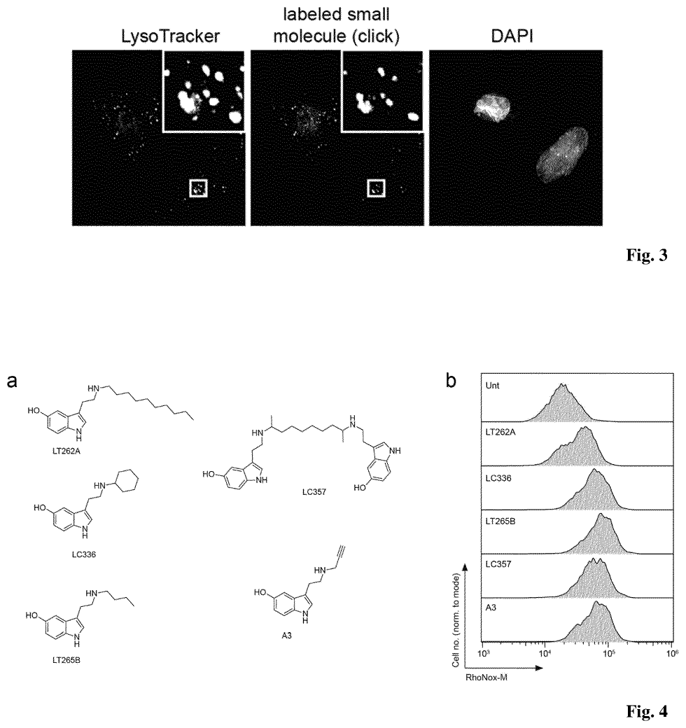

We treated cells with 1 μM of the serotonin analog A3 for 3 h and performed click chemistry using previously developed techniques by μs.^{1,2} Using a co-staining with LysoTracker, we found that these molecules accumulate in lysosomes (FIG. 3).

Using a lysosomotropic probe that detects Fe(II) specifically in lysosomes, called RhoNox-M,³ we saw an increase of RhoNox-M fluorescence measured by flow cytometry of cells treated with the serotonin analogs A3, LT262A, LC357, LC336 or LT265B (FIGS. 4 a,b). This data shows an increase of lysosomal Fe(II) upon treatment of cells with these compounds.

Treatment of cells with 100 nM A3 for 3 h or 24 h led to an increase of total intracellular iron and copper, measured by inductively coupled plasma mass spectrometry (FIGS. 5a,b).

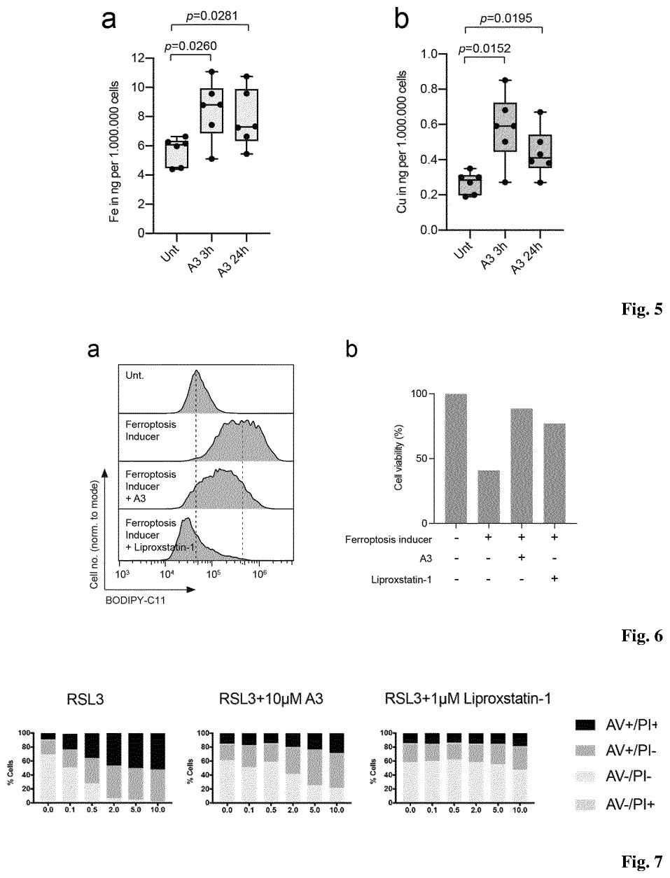

A3 led to a reduction of lipid peroxidation (measured by BODIPY-C11 fluorescence) induced by a ferroptosis inducer (FIG. 6a) and also to a rescue of cell viability due to toxicity caused by this ferroptosis inhibitor (FIG. 6b).

The serotonin analog A3 rescued cell death induced by the ferroptosis inducer RSL-3, with Liproxstatin-1 as a control in the same experiment (FIG. 7).

Taken together, these results demonstrate that the serotonin analogs form complexes with iron and copper, accumulate in lysosomes and can sequester lysosomal iron and copper. This leads to an increase of total intracellular iron and copper. The complexation of these metals protects the cells from oxidative stress and protects them against ferroptosis and cuproptosis.

### Example 3: Anti-Ferroptotic Activity

### Method:

Pancreatic Ductal Adenocarcinoma (PDAC) cells were grown at 37° C. and 5% CO₂ using medium prepared based on previously described (https://doi.org/10.1053/j.gastro.2004.03.058).

### Antibodies and Reagents

### IF: Immunofluorescence

Annexin/PI kit (Thermo Fisher Scientific, V13245, manufacturer's protocol), BODIPY-C11 (Thermo Fisher Scientific, D3861, manufacturer's protocol), Cyt c (Cell Signaling, 12963S, IF 1:200), EEA1 (Enzo Life Science, ALX210-239, IF 1:200), LysoTracker (LysoTracker Deep Red, Thermo Fisher Scientific, L12492, 45 min on live cells before fixation), Rcas1 (Cell Signaling, 12290, IF 1:200), PDIA3 (Sigma-Aldrich, AMAB90988, IF 1:200), RSL3 (Sigma-Aldrich, SML2234-5 MG).

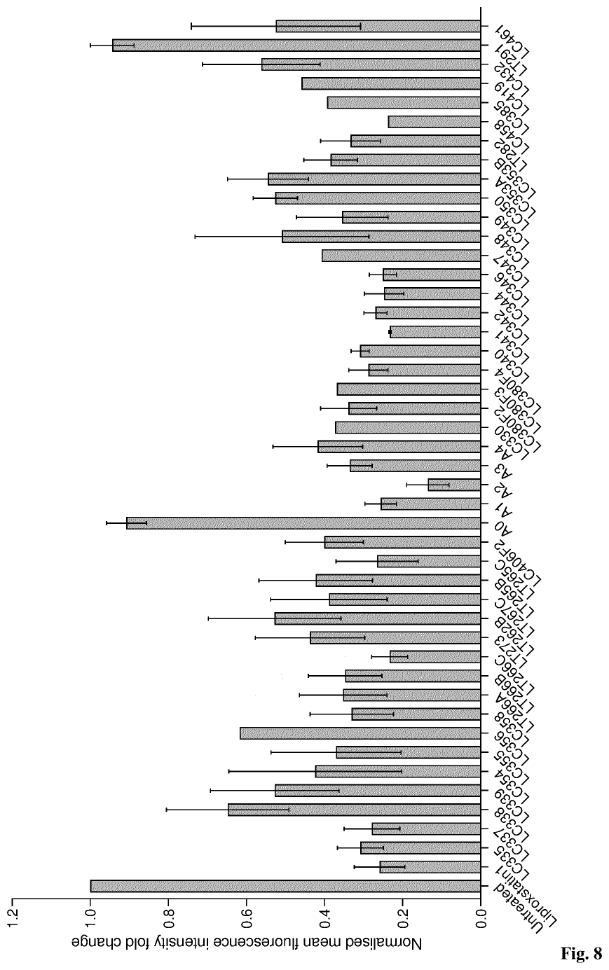

### Anti-Ferroptotic Activity (FIG. 8)

PDAC cells were seeded on 6 well plates at the density of 2×10⁵ cells/well. RSL3 (200 nM) was added along with compounds (10 μM for screening) on the following day and after 23 hr, BODIPY (4 μM) was added for 1 hr. The media was removed and the cells were washed with PBS twice before typsinization. The cells were harvested, pelleted, washed with PBS and finally PBS buffer containing 10% FBS and EDTA, was added for flow cytometry. The results are shown in FIG. 8 and this enabled us to establish a robust structure activity relationship on the serotonin scaffold.

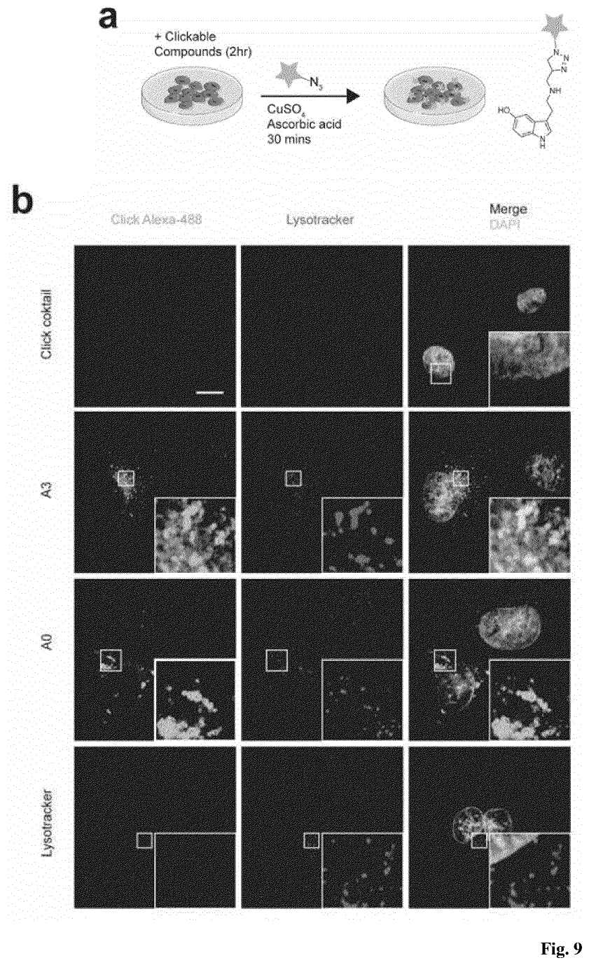

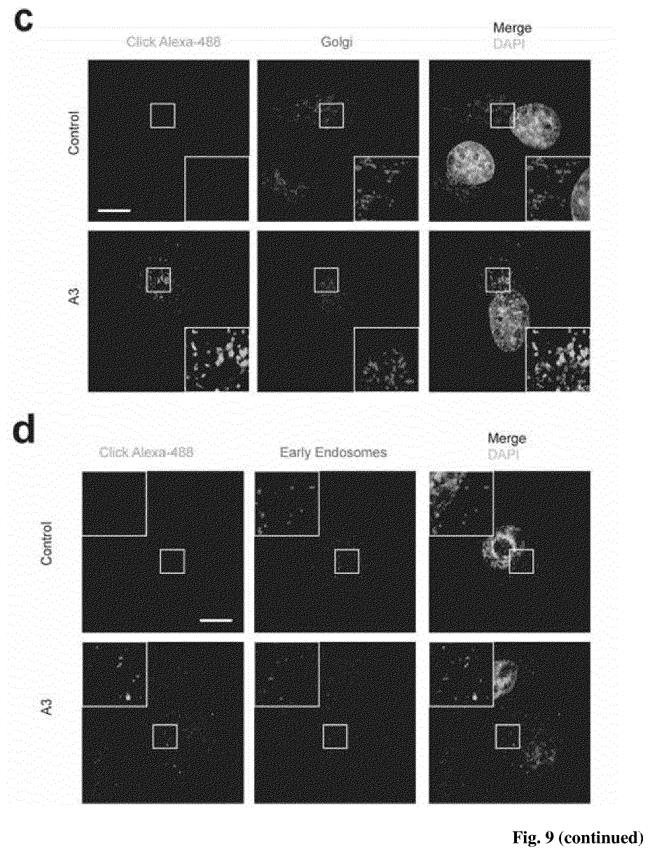

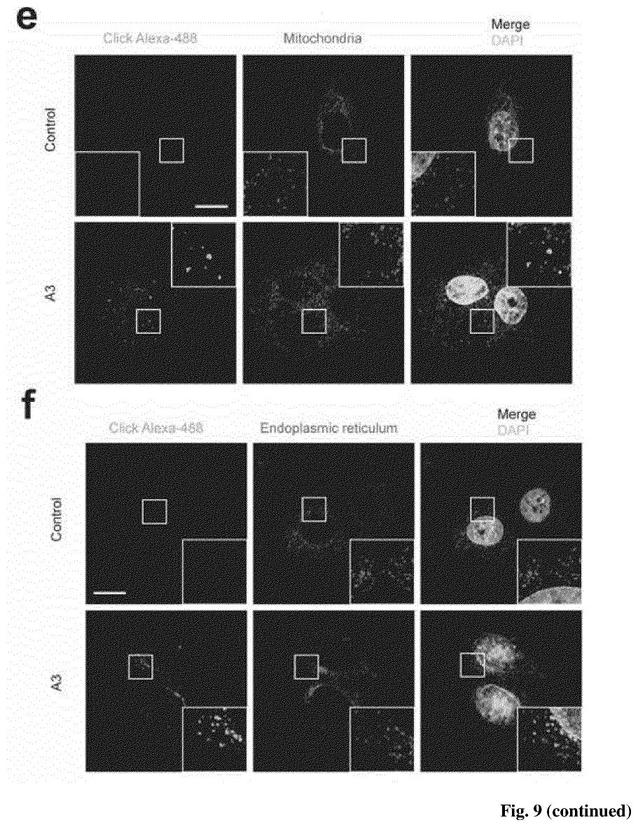

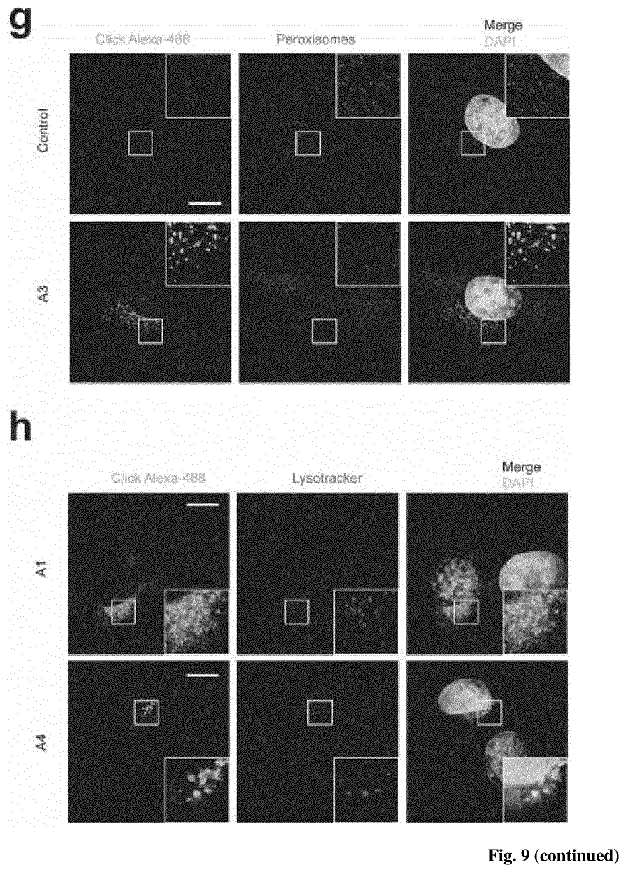

### Fluorescence Microscopy (FIG. 9)

Cells were plated on cover slips 24 h prior to the experiment. The cells were treated with 10 UM of compounds for 3 h and LysoTracker Deep Red (ThermoFisher Scientific L12492, 1:10000) according to the manufacturer's protocol. Subsequently, cells were washed three times with 1×PBS and fixed with 2% paraformaldehyde (w/v) in 1×PBS for 12 min, and then washed three times with 1×PBS. After fixation, cells were permeabilized with 0.1% Triton X-100 (v/v) in 1×PBS for 5 min and washed three times with 1×PBS. Then, cells were blocked in 2% BSA, 0.2% Tween-20 (w/v)/1×PBS (blocking buffer) for 20 min at RT. The click reaction cocktail was prepared from Click-iT EdU Imaging Kits (ThermoFisher Scientific, C10337) according to the manufacturer's protocol. Briefly, 868 μL of 1× Click-iT reaction buffer was mixed with 40 μl CuSO₄ solution, 2 μL Alexa Fluor azide and 90 μl reaction buffer additive (sodium ascorbate) to reach a final volume of 1 mL. Cover slips were incubated with 50 μl of the click reaction cocktail in the dark at RT for 30 minutes, then washed three times with 1×PBS. Further, cells were incubated with the relevant antibody in blocking buffer for 1 h at room temperature and washed three times with blocking buffer. Then, cells were incubated with secondary antibodies for 1 h. Finally, cover slips were washed three times with 1×PBS and mounted using VECTASHIELD (Vector Laboratories) containing DAPI and were sealed with nail varnish Express manucure (Maybelline, 16P201). Fluorescence images were acquired using a Deltavision real-time microscope (Applied Precision). 100×/1.4NA objectives were used for 2D and 3D acquisitions that were deconvoluted with SoftWorx (Ratio conservative-10 iterations, Applied Precision) and processed with ImageJ.

The results are shown in FIG. 9 which reveals that A3 goes to lysosomes in cells and it doesn't colocalize with other cell organelles. Active compounds A1 and A4 are also localized in lysosomes and inactive compound A0 doesn't go to lysosomes, indicating importance of lysosomes in targeting ferroptosis.

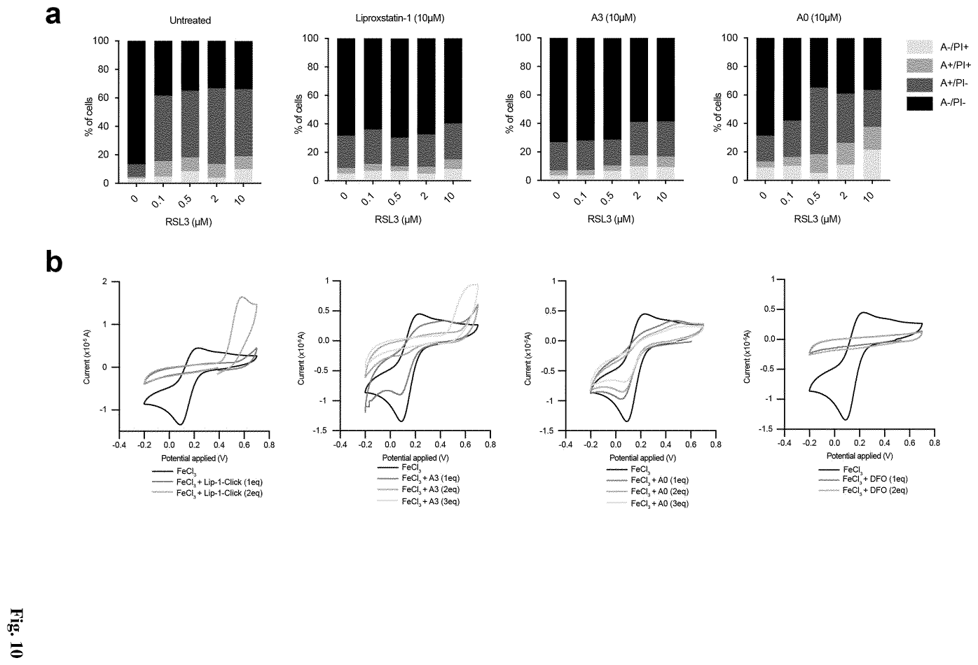

Cell Apoptosis Assay with Annexin V & Propidium Iodide (PI) (FIG. 10a)

PDAC cells were seeded on 6 well plates at the density of 2×10⁵ cells/well. RSL3 (0.1, 0.5, 2, 10 UM) was added along with compounds (10 μM) on the following day and after 24 h, the media was recovered and the cells were typsinated. The cells were harvested, pelleted along with the media recovered, washed with PBS, and 100 μl of 1× annexin binding buffer containing annexin V and PI (according to the manufacturer's protocol Dead Cell Apoptosis Kits with Annexin V for Flow Cytometry (Invitrogen)). Finally, PBS buffer containing 10% FBS and 1 mM of EDTA, was added for flow cytometry.

FIG. 10a. shows flow cytometry profiles of Annexin/propidium iodide of cells treated with various concentrations of RSL3 and co-treated with A3, A0 or Liproxstatiin-1 and their quantification. A3 protects against RSL3-induced ferroptosis.

### Cyclic Voltammetry Experiment (FIG. 10b)

Cyclic voltammetry experiments were performed with a three-electrode cell under argon. Saturated Calomel Electrode (SCE) was used as reference, a steady Glassy Carbon (GC) electrode of diameter 3 mm was selected as working electrode and a Platinum wire as counter-electrode. All cyclic voltammograms were recorded at RT with a μ-autolab III from Metrohm using Nova software with a scan rate of 2 V/s. MeCN was used as a degassed HPLC grade from Carlo Erba. Water was mQ H2O. Stock solution of FeCl₃ (100 mM) and Lip-1, A3, A0 and DFO (20 mM) were prepared with 0.3 M nBu4NBF4 in MeCN. 1 mM FeCl₃ in 0.3 M nBu4NBF4 in MeCN (1 mL) was used as working solution for recording and subsequently equivalents of compounds were added, stirred and recorded.

FIG. 10b. shows cyclic voltammetry curve of FeCl₃ and after addition of compounds. This shows that A3 chelates/binds to Fe³⁺ similar to the known Fe³⁺ chelator-DFO, as opposed to inactive compound A0 which doesn't binds to Fe³⁺.

### REFERENCES

1 Cañeque, T., Müller, S. & Rodriguez, R. Visualizing biologically active small molecules in cells using click chemistry. Nat. Rev. Chem. 2, 202-215 (2018).
2 Mai, T. T. et al. Salinomycin kills cancer stem cells by sequestering iron in lysosomes. Nat. Chem. 9, 1025-1033 (2017).
3 Niwa, M., Hirayama, T., Okuda, K. & Nagasawa, H. A new class of high-contrast Fe(II) selective fluorescent probes based on spirocyclized scaffolds for visualization of intracellular labile iron delivered by transferrin. Org. Biomol. Chem. 12, 6590-6597 (2014).

1-18. (canceled)

19. A method of treating metalloptosis associated disorders, comprising administering a compound of formula (I), or a pharmaceutically acceptable salt and/or solvate thereof, to a subject in need of treatment, wherein formula (I) is: 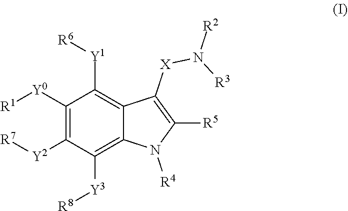
wherein
R¹, R², R³ and R⁴ are independently selected from the group consisting of H, —OH, —C(O)OR⁹—, —S(O)₂R¹⁰—, —C(O)R⁹—, —S(O)R¹⁰—, —NR¹¹R¹²—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl, provided that at least one of R¹, R² and R³ is not H; or R¹ and R⁴ are independently selected from the group consisting of H, —OH, —C(O)OR⁹—, —S(O)₂R¹⁰—, —C(O)R⁹—, —S(O)R¹⁰—, —NR¹¹R¹²—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl, and R² and R³ form together with the nitrogen atom to which they bound an optionally substituted heterocycloalkyl;
X is selected from the group consisting of (C₁-C₁₂)alkyl, —O—(C₁-C₁₂)alkyl-, —C(O)—, —C(O)—(C₁-C₁₂)alkyl-, —NH—C(O)—(C₁-C₁₂)alkyl-, —S—(C₁-C₁₂)alkyl-, —S(O)—(C₁-C₁₂)alkyl-, and —S(O)₂—(C₁-C₁₂)alkyl-; the (C₁-C₁₂)alkyl groups being optionally substituted and/or optionally interrupted by an heteroatom;
Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—, —C(O)NR¹⁴—, (C₁-C₂₄)alkyl, —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴—, —NH—C(O)—NH—, —NH—S(O)—NH—, —NH—S(O₂)—NH—, —S—C(O)—NH—, —O—C(O)—NH—, —O—S(O)—NH—, —O—S(O₂)—NH—, —S—CH₂—C(O)—NH—, —S—CH₂—S(O)—NH—, —S—CH₂—S(O₂)—NH—, —S(O)—CH₂—C(O)—NH—, —S(O₂)—CH₂—C(O)—NH—, —O—CH₂—C(O)—NH—, —O—CH₂—S(O)—NH—, —O—CH₂—S(O₂)—NH—, —NH—CH₂—C(O)—NH—, —NH—CH₂—S(O)—NH—, —NH—CH₂—S(O₂)—NH—, —O—(CH₂)₂—NH—C(O)—, —O—(CH₂)₂—N(CH₃)—C(O)—, —O—(CH₂)₂—NH—S(O)—, —O—(CH₂)₂—N(CH₃)—S(O)—, —O—(CH₂)₂—NH—S(O₂)—, —O—(CH₂)₂—N(CH₃)—S(O₂)—, —S—(CH₂)₂—NH—C(O)—, —S—(CH₂)₂—N(CH₃)—C(O)—, —S—(CH₂)₂—NH—S(O)—, —S—(CH₂)₂—N(CH₃)—S(O)—, —S—(CH₂)₂—NH—S(O₂)—, —S—(CH₂)₂—N(CH₃)—S(O₂)—, —NH—(CH₂)₂—NH—C(O)—, —NH—(CH₂)₂—N(CH₃)—C(O)—, —NH—(CH₂)₂—NH—S(O)—, —NH—(CH₂)₂—N(CH₃)—S(O)—, —NH—(CH₂)₂—NH—S(O₂)—, —NH—(CH₂)₂—N(CH₃)—S(O₂)—, —O—CH₂—CH(CH₂—OH)—NH—C(O)—, —O—CH₂—CH(CH₂—OCH₃)—NH—C(O)—, —O—CH₂—CH(CH₃)—NH—C(O)—, —O—CH(CH₃)—CH₂—NH—C(O)—, —O—CH₂—CH(CH₂—OH)—NH—S(O)—, —O—CH₂—CH(CH₂—OCH₃)—NH—S(O)—, —O—CH₂—CH(CH₃)—NH—S(O)—, —O—CH(CH₃)—CH₂—NH—S(O)—, —O—CH₂—CH(CH₂—OH)—NH—S(O₂)—, —O—CH₂—CH(CH₂—OCH₃)—NH—S(O₂)—, —O—CH₂—CH(CH₃)—NH—S(O₂)—, —O—CH(CH₃)—CH₂—NH—S(O₂)—, —S—CH₂—CH(CH₂—OH)—NH—C(O)—, —S—CH₂—CH(CH₂—OCH₃)—NH—C(O)—, —S—CH₂—CH(CH₃)—NH—C(O)—, —S—CH(CH₃)—CH₂—NH—C(O)—, —S—CH₂—CH(CH₂—OH)—NH—S(O)—, —S—CH₂—CH(CH₂—OCH₃)—NH—S(O)—, —S—CH₂—CH(CH₃)—NH—S(O)—, —S—CH(CH₃)—CH₂—NH—S(O)—, —S—CH₂—CH(CH₂—OH)—NH—S(O₂)—, —S—CH₂—CH(CH₂—OCH₃)—NH—S(O₂)—, —S—CH₂—CH(CH₃)—NH—S(O₂)—, —S—CH(CH₃)—CH₂—NH—S(O₂)—, —NH—CH₂—CH(CH₂—OH)—NH—C(O)—, —NH—CH₂—CH(CH₂—OCH₃)—NH—C(O)—, —NH—CH₂—CH(CH₃)—NH—C(O)—, —NH—CH(CH₃)—CH₂—NH—C(O)—, —NH—CH₂—CH(CH₂—OH)—NH—S(O)—, —NH—CH₂—CH(CH₂—OCH₃)—NH—S(O)—, —NH—CH₂—CH(CH₃)—NH—S(O)—, —NH—CH(CH₃)—CH₂—NH—S(O)—, —NH—CH₂—CH(CH₂—OH)—NH—S(O₂)—, —NH—CH₂—CH(CH₂—OCH₃)—NH—S(O₂)—, —NH—CH₂—CH(CH₃)—NH—S(O₂)—, —NH—CH(CH₃)—CH₂—NH—S(O₂)—, —O—(CH₂)₂—NH—, —S—(CH₂)₂—NH—, and —NH—(CH₂)₂—NH—; the (C₁-C₂₄)alkyl groups being optionally substituted; and
R⁵, R⁶, R⁷ and R⁸ are independently selected from the group consisting of H, halogen, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl; and
R⁹ to R¹⁴ are independently selected from the group consisting of optionally substituted (C₁-C₁₂)alkyl, optionally substituted (C₂-C₁₂) alkenyl, optionally substituted (C₂-C₁₂) alkynyl, optionally substituted aryl.

20. The method according to claim 19, wherein the metalloptosis associated disorders are disorders associated to ferroptosis and/or cuproptosis.

21. The method according to claim 19, wherein the metalloptosis associated disorders are selected from the group consisting of Hemorrhagic stroke; Ischemic Heart Injury; Ischemic Renal Injury; Ischaemia-reperfusion injury (IRI); Neurodegenerative diseases selected from Huntington Disease, Dementia, and amyotrophic lateral sclerosis (ALS); aging related diseases or disorders; sepsis; ishemia-reperfusion; and Diabetes.

22. The method according to claim 19, wherein said compound is selected from compounds recited in Table 1, or a pharmaceutically acceptable salt and/or solvate thereof.

23. A compound of general formula (I): 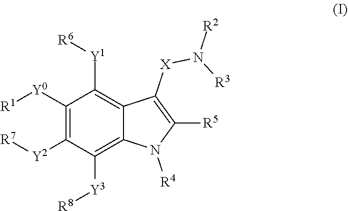
or a pharmaceutically acceptable salt and/or solvate thereof,
wherein
R¹, R², R³ and R⁴ are independently selected from the group consisting of H, OH, —C(O)OR⁹—, —S(O)₂R¹⁰—, —C(O)R⁹—, —S(O)R¹⁰—, —NR¹¹R¹²—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl, provided that at least one of R² and R³ is not H; or R¹ and R⁴ are independently selected from the group consisting of H, OH, —C(O)OR⁹—, —S(O)₂R¹⁰—, —C(O)R⁹—, —S(O)R¹⁰—, —NR¹¹R¹²—, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl, and R² and R³ with the nitrogen atom to which they are bound form together a nitrogen-heterocycloalkyl optionally substituted with at least one substituent selected from the group consisting of halogen; CN; NO₂; (C₁-C₆)alkyl; (C₁-C₆)haloalkyl; (C₁-C₆)haloalkoxy, (C₁-C₆)halothioalkoxy, optionally substituted aryl, an aryl substituted with one or two (C₁-C₆)alkyl or NO₂; heteroaryl; oxo; NR^{a}R^{b}; COR^{c}; CO₂R^{d}; CONR^{e}R^{f}; OR^{g}; SR^{h}, SO₂Rⁱ; SO₂NR^{j}R^{k}; NR^{l}COR^{m}; RⁿNR^{O}R^{P}; NR^{q}SO₂R^{r}; wherein R^{a} to R^{r} are, independently of one another, H, (C₁-C₆)alkyl, (C₁-C₆)haloalkyl, aryl, (C₁-C₆)alkyl-aryl, heteroaryl or (C₁-C₆)alkyl-heteroaryl;
Y⁰, Y¹, Y² and Y³ are independently absent or selected from —NH—, —O—, —S—, —S(O)—, —S(O)₂—, —C(O)—, —C(O)O—, —OC(O)—, —NHC(O)—, —C(O)NH—, —C(O)NR¹⁴—, —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴—, (C₁-C₂₄)alkyl, —NH—C(O)—NH—, —NH—S(O)—NH—, —NH—S(O₂)—NH—, —S—C(O)—NH—, —O—C(O)—NH—, —O—S(O)—NH—, —O—S(O₂)—NH—, —S—CH₂—C(O)—NH—, —S—CH₂—S(O)—NH—, —S—CH₂—S(O₂)—NH—, —S(O)—CH₂—C(O)—NH—, —S(O₂)—CH₂—C(O)—NH—, —O—CH₂—C(O)—NH—, —O—CH₂—S(O)—NH—, —O—CH₂—S(O₂)—NH—, —NH—CH₂—C(O)—NH—, —NH—CH₂—S(O)—NH—, —NH—CH₂—S(O₂)—NH—, —O—(CH₂)₂—NH—C(O)—, —O—(CH₂)₂—N(CH₃)—C(O)—, —O—(CH₂)₂—NH—S(O)—, —O—(CH₂)₂—N(CH₃)—S(O)—, —O—(CH₂)₂—NH—S(O₂)—, —O—(CH₂)₂—N(CH₃)—S(O₂)—, —S—(CH₂)₂—NH—C(O)—, —S—(CH₂)₂—N(CH₃)—C(O)—, —S—(CH₂)₂—NH—S(O)—, —S—(CH₂)₂—N(CH₃)—S(O)—, —S—(CH₂)₂—NH—S(O₂)—, —S—(CH₂)₂—N(CH₃)—S(O₂)—, —NH—(CH₂)₂—NH—C(O)—, —NH—(CH₂)₂—N(CH₃)—C(O)—, —NH—(CH₂)₂—NH—S(O)—, —NH—(CH₂)₂—N(CH₃)—S(O)—, —NH—(CH₂)₂—NH—S(O₂)—, —NH—(CH₂)₂—N(CH₃)—S(O₂)—, —O—CH₂—CH(CH₂—OH)—NH—C(O)—, —O—CH₂—CH(CH₂—OCH₃)—NH—C(O)—, —O—CH₂—CH(CH₃)—NH—C(O)—, —O—CH(CH₃)—CH₂—NH—C(O)—, —O—CH₂—CH(CH₂—OH)—NH—S(O)—, —O—CH₂—CH(CH₂—OCH₃)—NH—S(O)—, —O—CH₂—CH(CH₃)—NH—S(O)—, —O—CH(CH₃)—CH₂—NH—S(O)—, —O—CH₂—CH(CH₂—OH)—NH—S(O₂)—, —O—CH₂—CH(CH₂—OCH₃)—NH—S(O₂)—, —O—CH₂—CH(CH₃)—NH—S(O₂)—, —O—CH(CH₃)—CH₂—NH—S(O₂)—, —S—CH₂—CH(CH₂—OH)—NH—C(O)—, —S—CH₂—CH(CH₂—OCH₃)—NH—C(O)—, —S—CH₂—CH(CH₃)—NH—C(O)—, —S—CH(CH₃)—CH₂—NH—C(O)—, —S—CH₂—CH(CH₂—OH)—NH—S(O)—, —S—CH₂—CH(CH₂—OCH₃)—NH—S(O)—, —S—CH₂—CH(CH₃)—NH—S(O)—, —S—CH(CH₃)—CH₂—NH—S(O)—, —S—CH₂—CH(CH₂—OH)—NH—S(O₂)—, —S—CH₂—CH(CH₂—OCH₃)—NH—S(O₂)—, —S—CH₂—CH(CH₃)—NH—S(O₂)—, —S—CH(CH₃)—CH₂—NH—S(O₂)—, —NH—CH₂—CH(CH₂—OH)—NH—C(O)—, —NH—CH₂—CH(CH₂—OCH₃)—NH—C(O)—, —NH—CH₂—CH(CH₃)—NH—C(O)—, —NH—CH(CH₃)—CH₂—NH—C(O)—, —NH—CH₂—CH(CH₂—OH)—NH—S(O)—, —NH—CH₂—CH(CH₂—OCH₃)—NH—S(O)—, —NH—CH₂—CH(CH₃)—NH—S(O)—, —NH—CH(CH₃)—CH₂—NH—S(O)—, —NH—CH₂—CH(CH₂—OH)—NH—S(O₂)—, —NH—CH₂—CH(CH₂—OCH₃)—NH—S(O₂)—, —NH—CH₂—CH(CH₃)—NH—S(O₂)—, —NH—CH(CH₃)—CH₂—NH—S(O₂)—, —O—(CH₂)₂—NH—, —S—(CH₂)₂—NH—, and —NH—(CH₂)₂—NH—; the (C₁-C₂₄)alkyl groups being optionally substituted; and
R⁵, R⁶, R⁷ and R⁸ are independently selected from the group consisting of H, halogen, optionally substituted (C₁-C₂₄)alkyl, optionally substituted (C₂-C₂₄) alkenyl, optionally substituted (C₂-C₂₄) alkynyl, optionally substituted (C₃-C₁₀) cycloalkyl, optionally substituted heterocycloalkyl, optionally substituted aryl and optionally substituted heteroaryl; and
R⁹ to R¹⁴ are independently selected from the group consisting of optionally substituted (C₁-C₁₂)alkyl, optionally substituted (C₂-C₁₂) alkenyl, optionally substituted (C₂-C₁₂)alkynyl, optionally substituted aryl;
provided that when R⁵, R⁶, R⁷ and R⁸ are H, Y⁰ is —O—, and Y¹, Y² and Y³ are absent, then R¹, R² and R³ are not an optionally substituted (C₂-C₂₄) alkynyl.

24. The compound according to claim 23, wherein X is a (C₁-C₁₂)alkyl optionally substituted and/or optionally interrupted by a heteroatom, a (C₁-C₆)alkyl optionally substituted and/or optionally interrupted by an heteroatom, or an ethyl.

25. The compound according to claim 23, wherein R⁵, R⁶, R⁷ and R⁸ are independently selected from the group consisting of H, halogen, substituted (C₁-C₂₄)alkyl, optionally substituted (C₂-C₂₄) alkenyl, optionally substituted (C₂-C₂₄) alkynyl, haloalkyl, or (C₁-C₂₄)alkyl.

26. The compound according to claim 23, wherein R⁴ is H.

27. The compound according to claim 23, wherein R¹ is H.

28. The compound according to claim 23, wherein R² and R³ are independently selected from the group consisting of H, —C(O)OR⁹—, —S(O)₂R¹⁰—, optionally substituted (C₁-C₁₂)alkyl, optionally substituted (C₂-C₁₂) alkenyl, optionally substituted (C₂-C₁₂) alkynyl, optionally substituted (C₃-C₁₂) cycloalkyl, optionally substituted heterocycloalkyl, optionally substituted aryl and optionally substituted heteroaryl, provided that at least one of R² and R³ is not H; or R² and R³ with the nitrogen atom to which they are bound form together an optionally substituted nitrogen-heterocycloalkyl comprising 3 to 10 ring atoms.

29. The compound according to claim 23, wherein Y⁰, Y¹, Y² and Y³ are independently absent or -selected from the group consisting of NH—, —O—, —S—, —S(O)—, —S(O)₂—, —C(O)—, —C(O)O—, —OC(O)—, —NHC(O)—, —C(O)NH—, —C(O)NR¹⁴—, (C₁-C₂₄)alkyl, —(C₁-C₂₄)alkyl-NH—, —(C₁-C₂₄)alkyl-O—, —(C₁-C₂₄)alkyl-S—, (C₁-C₂₄)alkyl-NR¹³—, —(C₁-C₂₄)alkyl-S(O)—, —(C₁-C₂₄)alkyl-S(O)₂—, —(C₁-C₂₄)alkyl-C(O)—, —(C₁-C₂₄)alkyl-C(O)O—, —(C₁-C₂₄)alkyl-OC(O)—, —(C₁-C₂₄)alkyl-NHC(O)—, —(C₁-C₂₄)alkyl-C(O)NH—, —(C₁-C₂₄)alkyl-C(O)NR¹⁴—; the (C₁-C₂₄)alkyl groups being optionally substituted.

30. The compound according to claim 23, wherein:
a) Y⁰, Y¹, Y² and Y³ are independently absent or —O—; —NH—, —S— or optionally substituted (C₁-C₂₄)alkyl; or
b) Y⁰, Y¹, Y² and Y³ are independently absent or —O—; or
c) Y⁰ is —O— and Y¹, Y² and Y³ are independently absent or —O—.

31. The compound according to claim 23, wherein said compound is selected from:
a) compounds (001) to (064) of Table 1; or
b) compounds (002), (003), (004), (008), (009), (010), (012), (013), (014), (015), (016), (017), (018), (019), (020), (022), (023), (024), (025), (026), (028), (029), (036), (039), (040), (043), (045), (049), (051), (052), (054), (057), (058), (059), (061), (063), and (064) of Table 1; or
c) compounds (003) to (007), (013), (014), (021), (029) and (031) to (038) of Table 1;
or a pharmaceutically acceptable salt and/or solvate thereof.

32. A pharmaceutical composition comprising a compound according to claim 23 or a pharmaceutically acceptable salt and/or solvate thereof, and at least one pharmaceutically acceptable excipient.

33. A method of treating iron- and/or copper-associated disorders, iron and/or copper overload-associated disorders, or metal-dependent cell death associated disorders, comprising administering a compound according to claim 23, a pharmaceutically acceptable salt and/or solvate thereof, or a pharmaceutical composition comprising said compound, pharmaceutically acceptable salt and/or solvate of said compound, to a subject in need of treatment.

34. The method according to claim 33, wherein the iron- and/or copper-associated disorders are selected from HFE-related hematochromatosis; non HFE-related hematochromatosis; congenital atransferrinenemia; iron-loading associated anemias, thalassemia, myelodysplasia and hematopoietic stem-cell transplantation-associated disorders; chronic liver diseases; chronic inflammation linked to cancer; autoimmune or inflammatory diseases; neurodegeneration with brain iron accumulation-associated diseases, Wilson's Disease; polygenic neurodegenerative-associated diseases; Parkinson's Disease; and Alzheimer Disease.

35. The method according to claim 33, wherein the metal-dependent cell death associated disorders are metalloptosis associated disorders; disorders associated to ferroptosis and/or cuproptosis; Hemorrhagic stroke; Ischemic Heart Injury; Ischemic Renal Injury; Ischaemia-reperfusion injury (IRI); or Neurodegenerative diseases selected from Huntington Disease, Dementia, amyotrophic lateral and sclerosis (ALS); aging related diseases or disorders; sepsis; ishemia-reperfusion; and Diabetes.
